(19) United States
(12) Patent Application Publication
(10) Pub. No.: US 2022/0135875 A1
(43) Pub. Date: May 5, 2022

(54) PHOSPHOROUS HOST MATERIAL AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Young-Mook LIM, Gyeonggi-do (KR); Su-Hyun LEE, Gyeonggi-do (KR); Hyun-Ju KANG, Gyeonggi-do (KR); Chi-Sik KIM, Gyeonggi-do (KR)

(21) Appl. No.: 17/572,837

(22) Filed: Jan. 11, 2022

Related U.S. Application Data
(63) Continuation of application No. 15/573,167, filed on Nov. 10, 2017, now abandoned.

(30) Foreign Application Priority Data
May 19, 2015 (KR) 10-2015-0069699
Jun. 24, 2015 (KR) 10-2015-0089958
Jan. 7, 2016 (KR) 10-2016-0002155
Mar. 22, 2016 (KR) 10-2016-0034173

(51) Int. Cl. C09K 11/06; C07D 401/14; H01L 51/50; C07D 487/04; C07D 491/048; H01L 51/00; C07D 495/04; C07D 403/14; H05B 33/10

(52) U.S. Cl. CPC C09K 11/06; C09K 2211/1007; H01L 51/5016; C07D 487/04; C07D 491/048; H01L 51/0074; H01L 51/0072; C07D 495/04; C07D 403/14; H01L 51/0067; H01L 51/0073; H05B 33/10; C09K 2211/1044; C09K 2211/1037; C09K 2211/1033; C09K 2211/1029; C09K 2211/1059; C09K 2211/1011; C07D 401/14

(57) ABSTRACT

The present invention relates to phosphorous host materials and an organic electroluminescent device comprising the same. By using the phosphorous host material of the present invention, an organic electroluminescent device having significantly improved operational lifespan can be produced.

### CLAIM OF BENEFIT OF PRIOR APPLICATION

This application claims priority under 35 U.S.C. § 120 from U.S. patent application Ser. No. 15/573,167, filed Nov. 10, 2017, which is the National Stage Entry of PCT/KR2016/003716, filed Apr. 8, 2016, both of which are incorporated by reference herein in their entirety.

### TECHNICAL FIELD

The present invention relates to phosphorous host materials and organic electroluminescent device comprising the same.

### BACKGROUND ART

An electroluminescent device (EL device) is a self-light-emitting device which has advantages in that it provides a wider viewing angle, a greater contrast ratio, and a faster response time. The first organic EL device was developed by Eastman Kodak, by using small aromatic diamine molecules, and aluminum complexes as materials for forming a light-emitting layer [Appl. Phys. Lett. 51, 913, 1987].

The most important factor determining luminous efficiency in an organic EL device is light-emitting materials. Light-emitting materials are classified into fluorescent, phosphorous, and thermally activated delayed fluorescent (TADF) materials. In case of conventional carrier injection-type organic electroluminescent devices, electrons and holes injected from electrodes recombine at a light-emitting layer to form an exciton. Light is emitted as the exciton transfers to a ground state. Singlet state excitons and triplet state excitons are formed in a ratio of 1:3. A luminescence from the singlet state to the ground state is called fluorescence and a luminescence from the triplet state to the ground state is called phosphorous. It is difficult to observe phosphorous luminescence at room temperature in most of the organic compounds. However, when heavy element materials such as Ir, Pt, etc., are used, excitons in singlet states transfer to triplet states due to inter-system crossing (ISC), and all the excitons formed by recombination can be used in light-emission. Thus, 100% of internal quantum efficiency can be obtained.

Until now, fluorescent materials have been widely used as light-emitting material. However, in view of electroluminescent mechanisms, since phosphorescent materials theoretically enhance luminous efficiency by four (4) times compared to fluorescent materials, development of phosphorescent light-emitting materials is widely being researched. Iridium(III) complexes have been widely known as phosphorescent materials, including bis(2-(2′-benzothienyl)-pyridinato-N,C3′)iridium(acetylacetonate) ((acac)Ir(btp)₂), tris(2-phenylpyridine)iridium (Ir(ppy)₃) and bis(4,6-difluorophenylpyridinato-N,C2)picolinate iridium (Firpic) as red, green, and blue materials, respectively.

At present, 4,4′-N,N′-dicarbazol-biphenyl (CBP) is the most widely known phosphorescent host material. Recently, Pioneer (Japan) et al. developed a high performance organic EL device using bathocuproine (BCP) and aluminum(III)bis(2-methyl-8-quinolinate)(4-phenylphenolate) (BAlq) etc., as host materials, which were known as hole blocking layer materials.

Although these materials provide good light-emitting characteristics, they have the following disadvantages: (1) Due to their low glass transition temperature and poor thermal stability, their degradation may occur during a high-temperature deposition process in a vacuum, and the lifespan of the device decreases. (2) The power efficiency of an organic EL device is given by [(π/voltage)×current efficiency], and the power efficiency is inversely proportional to the voltage. Although an organic EL device comprising phosphorescent host materials provides higher current efficiency (cd/A) than one comprising fluorescent materials, a significantly high driving voltage is necessary. Thus, there is no merit in terms of power efficiency (lm/W). (3) Further, the operational lifespan of an organic EL device is short and luminous efficiency is still required to be improved.

According to a recent study, through a design of molecules having very little excitation energy difference between singlet state and triplet state, thermally activated delayed fluorescence in which cross between a force system from a triplet state to a singlet state is possible using thermal energy.

A prior art of KR 1477613 B1 discloses a compound in which pyridine, pyrimidine, or triazine is bonded to a nitrogen atom of carbazole fused with indole, directly or via a linker of phenylene. However, said reference does not specifically disclose a compound in which pyridine, pyrimidine, or triazine is bonded to a nitrogen atom of carbazole fused with indole via a linker of pyridylene or pyrimidinylene.

A prior art of KR 1317923 B1 discloses a compound in which pyridine is bonded to a nitrogen atom of carbazole fused with indole, via a linker of pyrimidinylene. However, said reference does not disclose any example using the compound as a phosphorous host material.

### DISCLOSURE OF THE INVENTION

### Problems to be Solved

The objective of the present invention is first, to provide a phosphorous host material capable of producing an organic electroluminescent device with excellent lifespan characteristics, and second, to provide an organic electroluminescent device comprising the phosphorous host material.

### Solution to Problems

The present inventors found that the above objective can be achieved by a phosphorous host material comprising a compound represented by the following formula 1:

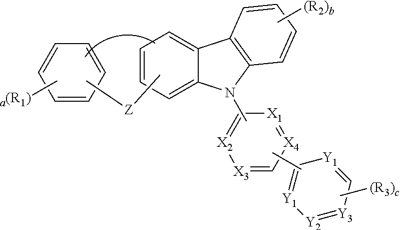

wherein
Z represents NR₄, CR₅R₆, O, or S;
X₁ to X₄ each independently represent N or C(R₇), one or more of which are N;
Y₁ to Y₃ each independently represent N or C(R₈), two or more of which are N;
R₁ to R₈ each independently represent hydrogen, deuterium, a halogen, a cyano, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur;
a and b each independently represent an integer of 1 to 4;
c represents 1 or 2;
where a, b, or c is an integer of 2 or more, each of R₁, each of R₂, or each of R₃ may be the same or different; and
the heteroaryl contains at least one hetero atom selected from B, N, O, S, Si, and P.

### Effects of the Invention

By using the phosphorous host material of the present invention, an organic electroluminescent device having significantly improved operational lifespan can be produced.

### EMBODIMENTS OF THE INVENTION

Hereinafter, the present invention will be described in detail. However, the following description is intended to explain the invention, and is not meant in any way to restrict the scope of the invention.

The present invention relates to a phosphorous host material comprising a compound represented by formula 1, and an organic electroluminescent device comprising the material.

Hereinafter, the compound represented by formula 1 will be described in detail.

The compound represented by formula 1 can be represented by one of the following formulas 2 to 6:

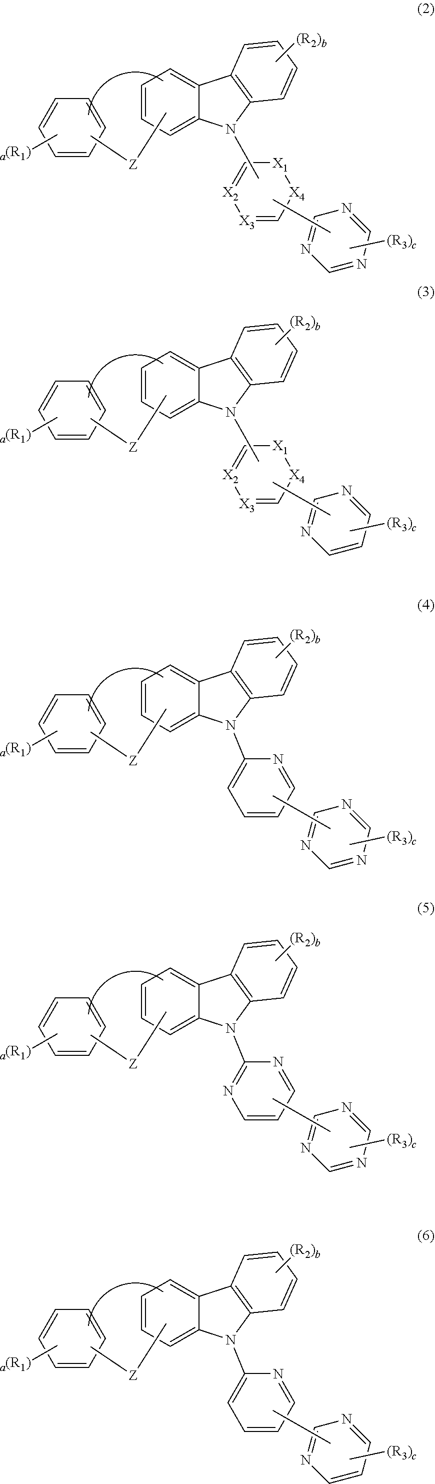

wherein
R₁ to R₃, X₁ to X₄, Z, and a to c are as defined in formula 1.

The structure of

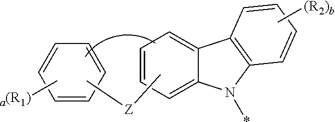

in formula 1 can be represented by one of the following formulas 7 to 12:

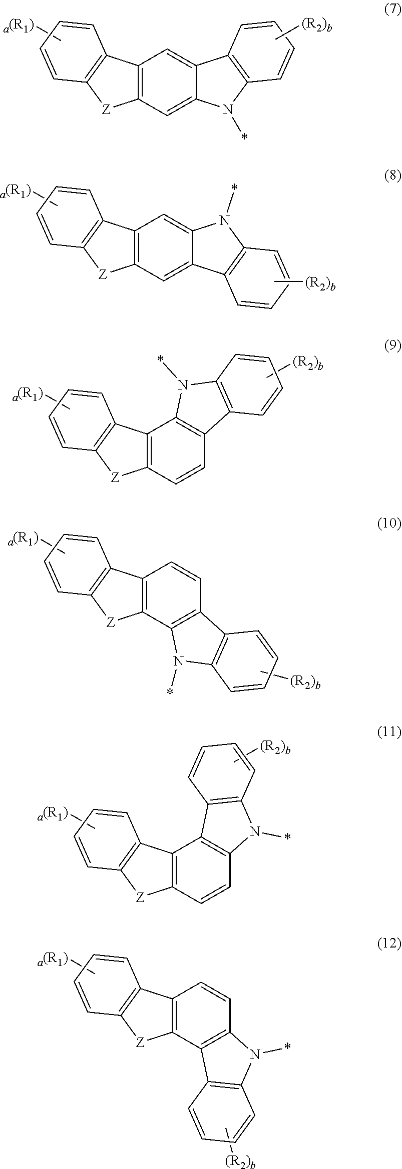

wherein
R₁, R₂, Z, a, and b are as defined in formula 1.

Herein, “(C1-C30)alkyl” is meant to be a linear or branched alkyl having 1 to 30 carbon atoms constituting the chain, in which the number of carbon atoms is preferably 1 to 10, more preferably 1 to 6, and includes methyl, ethyl, n-propyl, isopropyl, n-butyl, isobutyl, tert-butyl, etc.; “(C2-C30)alkenyl” is meant to be a linear or branched alkenyl having 2 to 30 carbon atoms constituting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc.; “(C2-C30)alkynyl” is meant to be a linear or branched alkynyl having 2 to 30 carbon atoms constituting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc.; “(C3-C30)cycloalkyl” is a mono- or polycyclic hydrocarbon having 3 to 30 ring backbone carbon atoms, in which the number of carbon atoms is preferably 3 to 20, more preferably 3 to 7, and includes cyclopropyl, cyclobutyl, cyclopentyl, cyclohexyl, etc.; “3- to 7-membered heterocycloalkyl” is a cycloalkyl having 3 to 7 ring backbone atoms, including at least one heteroatom selected from B, N, O, S, Si, and P, preferably O, S, and N, and includes tetrahydrofuran, pyrrolidine, thiolan, tetrahydropyran, etc.; “(C6-C30)aryl(ene)” is a monocyclic or fused ring derived from an aromatic hydrocarbon having 6 to 30 ring backbone carbon atoms, in which the number of carbon atoms is preferably 6 to 20, more preferably 6 to 15, and includes phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc.; “3- to 30-membered heteroaryl(ene)” is an aryl having 3 to 30 ring backbone atoms, including at least one, preferably 1 to 4 heteroatoms selected from the group consisting of B, N, O, S, Si, and P; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br, and I.

Herein, “substituted” in the expression, “substituted or unsubstituted,” means that a hydrogen atom in a certain functional group is replaced with another atom or group, i.e. a substituent. In the present invention, the substituents of the substituted (C1-C30)alkyl, the substituted (C6-C30)aryl, the substituted 3- to 30-membered heteroaryl, the substituted (C3-C30)cycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, the substituted (C1-C30)alkyl(C6-C30)arylamino, and the substituted mono- or polycyclic, (C3-C30) alicyclic or aromatic ring in R₁ to R₈ in formula 1 each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 5- to 30-membered heteroaryl unsubstituted or substituted with a (C6-C30)aryl, a (C6-C30)aryl unsubstituted or substituted with a 5- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

Z represents NR₄, CR₅R₆, O, or S.

X₁ to X₄ each independently represent N or C(R₇), one or more of which are N.

Y₁ to Y₃ each independently represent N or C(R₈), two or more of which are N.

R₁ to R₈ each independently represent hydrogen, deuterium, a halogen, a cyano, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur; preferably each independently represent hydrogen, a substituted or unsubstituted (C1-C6)alkyl, or a substituted or unsubstituted (C6-C15)aryl; or are linked to an adjacent substituent(s) to form a substituted or unsubstituted, mono- or polycyclic, (C3-C12) aromatic ring; and more preferably R₁ and R₂ each independently represent hydrogen; or are linked to an adjacent substituent(s) to form a substituted or unsubstituted, monocyclic (C3-C12) aromatic ring, R₃ to R₆ each independently represent an unsubstituted (C1-C6)alkyl, or a (C6-C15)aryl unsubstituted or substituted with a (C1-C6)alkyl, and R₇ and R₈ each independently represent hydrogen.

According to one embodiment of the present invention, in formula 1 above, Z represents NR₄, CR₅R₆, O, or S; X₁ to X₄ each independently represent N or C(R₇), one or more of which are N; Y₁ to Y₃ each independently represent N or C(R₈), two or more of which are N; and R₁ to R₈ each independently represent hydrogen, a substituted or unsubstituted (C1-C6)alkyl, or a substituted or unsubstituted (C6-C15)aryl; or are linked to an adjacent substituent(s) to form a substituted or unsubstituted, mono- or polycyclic, (C3-C12) aromatic ring.

According to another embodiment of the present invention, in formula 1 above, Z represents NR₄, CR₅R₆, O, or S; X₁ to X₄ each independently represent N or C(R₇), one or more of which are N; Y₁ to Y₃ each independently represent N or C(R₈), two or more of which are N; R₁ and R₂ each independently represent hydrogen; or are linked to an adjacent substituent(s) to form a substituted or unsubstituted, monocyclic (C3-C12) aromatic ring; R₃ to R₆ each independently represent an unsubstituted (C1-C6)alkyl, or a (C6-C15)aryl unsubstituted or substituted with a (C1-C6)alkyl; and R₇ and R₈ each independently represent hydrogen.

The compound represented by formula 1 includes the following compounds, but is not limited thereto:

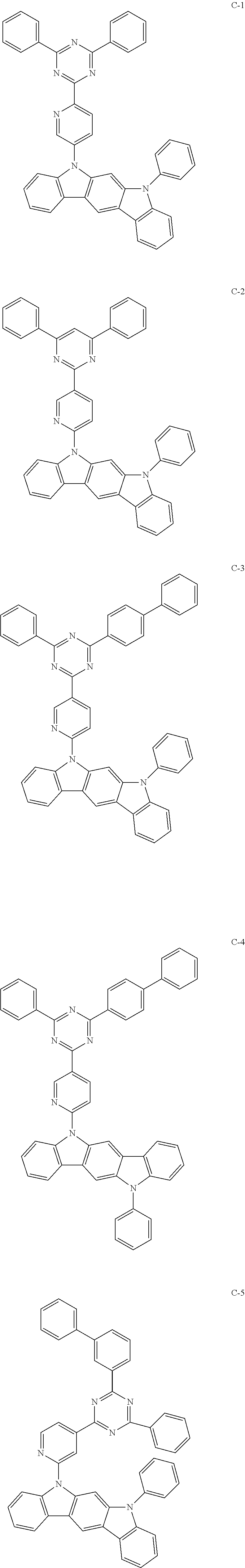 [SMILES: c1ccc(-c2nc(-c3ccccc3)nc(-c3ccc(-n4c5ccccc5c5cc6c7ccccc7n(-c7ccccc7)c6cc54)cn3)n2)cc1 ; c1ccc(-c2cc(-c3ccccc3)nc(-c3ccc(-n4c5ccccc5c5cc6c7ccccc7n(-c7ccccc7)c6cc54)nc3)n2)cc1 ; c1ccc(-c2ccc(-c3nc(-c4ccccc4)nc(-c4ccc(-n5c6ccccc6c6cc7c8ccccc8n(-c8ccccc8)c7cc65)nc4)n3)cc2)cc1 ; c1ccc(-c2ccc(-c3nc(-c4ccccc4)nc(-c4ccc(-n5c6ccccc6c6cc7c(cc65)c5ccccc5n7-c5ccccc5)nc4)n3)cc2)cc1 ; c1ccc(-c2cccc(-c3nc(-c4ccccc4)nc(-c4ccnc(-n5c6ccccc6c6cc7c8ccccc8n(-c8ccccc8)c7cc65)c4)n3)c2)cc1]

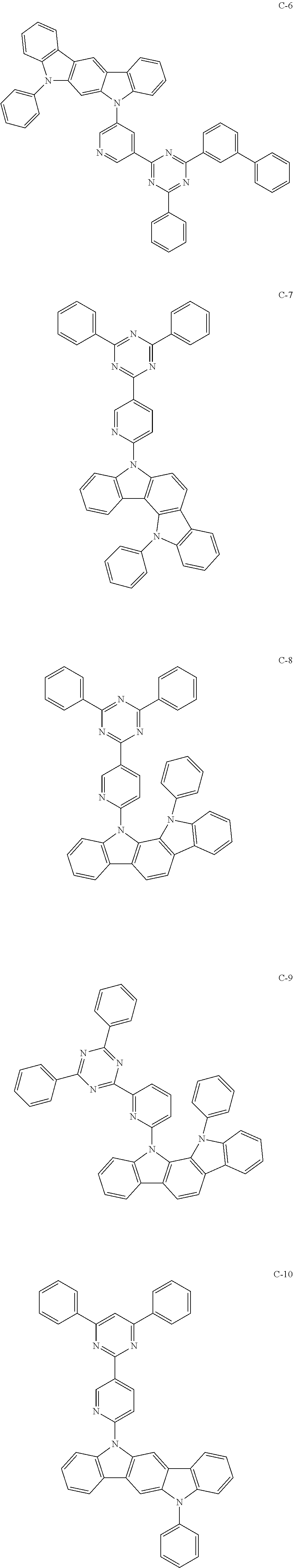 [SMILES: c1ccc(-c2cccc(-c3nc(-c4ccccc4)nc(-c4cncc(-n5c6ccccc6c6cc7c8ccccc8n(-c8ccccc8)c7cc65)c4)n3)c2)cc1 ; c1ccc(-c2nc(-c3ccccc3)nc(-c3ccc(-n4c5ccccc5c5c4ccc4c6ccccc6n(-c6ccccc6)c45)nc3)n2)cc1 ; c1ccc(-c2nc(-c3ccccc3)nc(-c3ccc(-n4c5ccccc5c5ccc6c7ccccc7n(-c7ccccc7)c6c54)nc3)n2)cc1 ; c1ccc(-c2nc(-c3ccccc3)nc(-c3cccc(-n4c5ccccc5c5ccc6c7ccccc7n(-c7ccccc7)c6c54)n3)n2)cc1 ; c1ccc(-c2cc(-c3ccccc3)nc(-c3ccc(-n4c5ccccc5c5cc6c(cc54)c4ccccc4n6-c4ccccc4)nc3)n2)cc1]

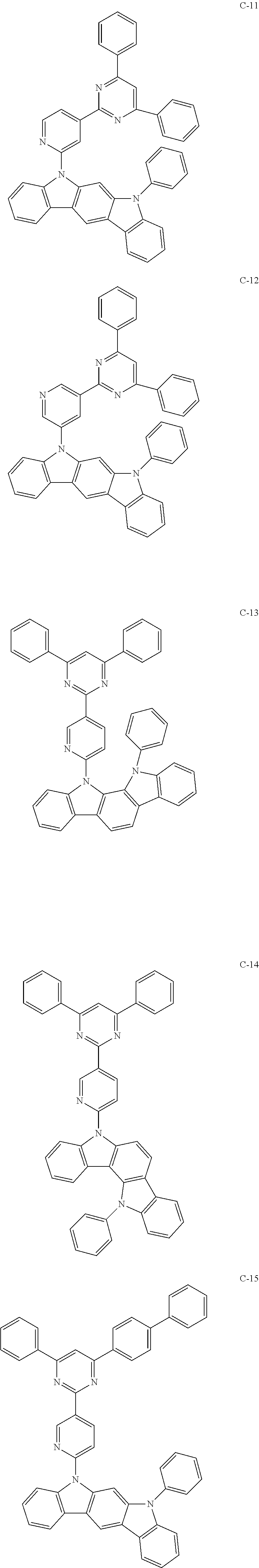 [SMILES: c1ccc(-c2cc(-c3ccccc3)nc(-c3ccnc(-n4c5ccccc5c5cc6c7ccccc7n(-c7ccccc7)c6cc54)c3)n2)cc1 ; c1ccc(-c2cc(-c3ccccc3)nc(-c3cncc(-n4c5ccccc5c5cc6c7ccccc7n(-c7ccccc7)c6cc54)c3)n2)cc1 ; c1ccc(-c2cc(-c3ccccc3)nc(-c3ccc(-n4c5ccccc5c5ccc6c7ccccc7n(-c7ccccc7)c6c54)nc3)n2)cc1 ; c1ccc(-c2cc(-c3ccccc3)nc(-c3ccc(-n4c5ccccc5c5c4ccc4c6ccccc6n(-c6ccccc6)c45)nc3)n2)cc1 ; c1ccc(-c2ccc(-c3cc(-c4ccccc4)nc(-c4ccc(-n5c6ccccc6c6cc7c8ccccc8n(-c8ccccc8)c7cc65)nc4)n3)cc2)cc1]

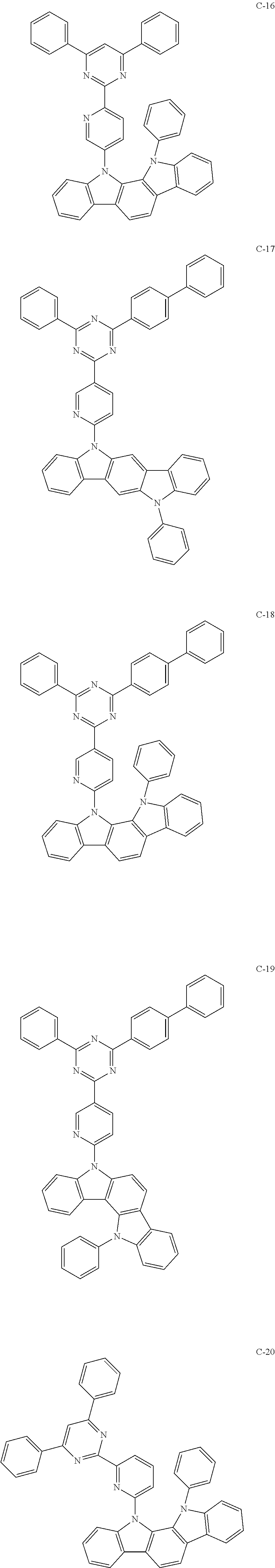 [SMILES: c1ccc(-c2cc(-c3ccccc3)nc(-c3ccc(-n4c5ccccc5c5ccc6c7ccccc7n(-c7ccccc7)c6c54)cn3)n2)cc1 ; c1ccc(-c2ccc(-c3nc(-c4ccccc4)nc(-c4ccc(-n5c6ccccc6c6cc7c(cc65)c5ccccc5n7-c5ccccc5)nc4)n3)cc2)cc1 ; c1ccc(-c2ccc(-c3nc(-c4ccccc4)nc(-c4ccc(-n5c6ccccc6c6ccc7c8ccccc8n(-c8ccccc8)c7c65)nc4)n3)cc2)cc1 ; c1ccc(-c2ccc(-c3nc(-c4ccccc4)nc(-c4ccc(-n5c6ccccc6c6c5ccc5c7ccccc7n(-c7ccccc7)c56)nc4)n3)cc2)cc1 ; c1ccc(-c2cc(-c3ccccc3)nc(-c3cccc(-n4c5ccccc5c5ccc6c7ccccc7n(-c7ccccc7)c6c54)n3)n2)cc1]

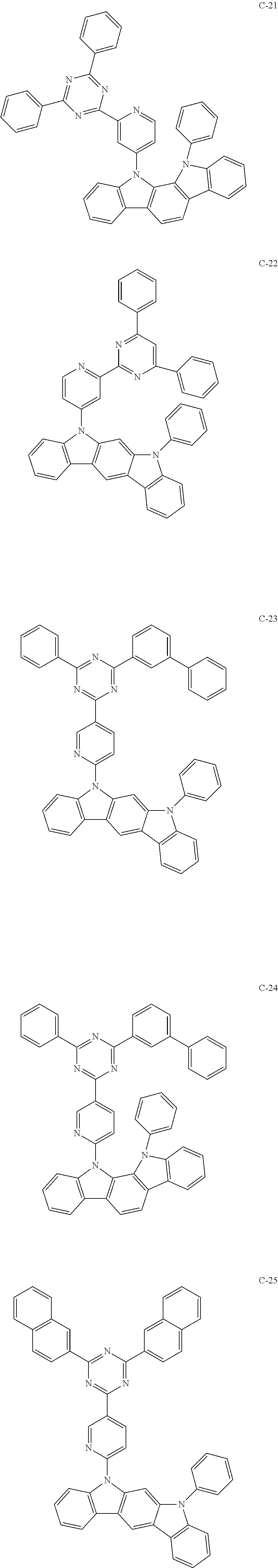 [SMILES: c1ccc(-c2nc(-c3ccccc3)nc(-c3cc(-n4c5ccccc5c5ccc6c7ccccc7n(-c7ccccc7)c6c54)ccn3)n2)cc1 ; c1ccc(-c2cc(-c3ccccc3)nc(-c3cc(-n4c5ccccc5c5cc6c7ccccc7n(-c7ccccc7)c6cc54)ccn3)n2)cc1 ; c1ccc(-c2cccc(-c3nc(-c4ccccc4)nc(-c4ccc(-n5c6ccccc6c6cc7c8ccccc8n(-c8ccccc8)c7cc65)nc4)n3)c2)cc1 ; c1ccc(-c2cccc(-c3nc(-c4ccccc4)nc(-c4ccc(-n5c6ccccc6c6ccc7c8ccccc8n(-c8ccccc8)c7c65)nc4)n3)c2)cc1 ; c1ccc(-n2c3ccccc3c3cc4c5ccccc5n(-c5ccc(-c6nc(-c7ccc8ccccc8c7)nc(-c7ccc8ccccc8c7)n6)cn5)c4cc32)cc1]

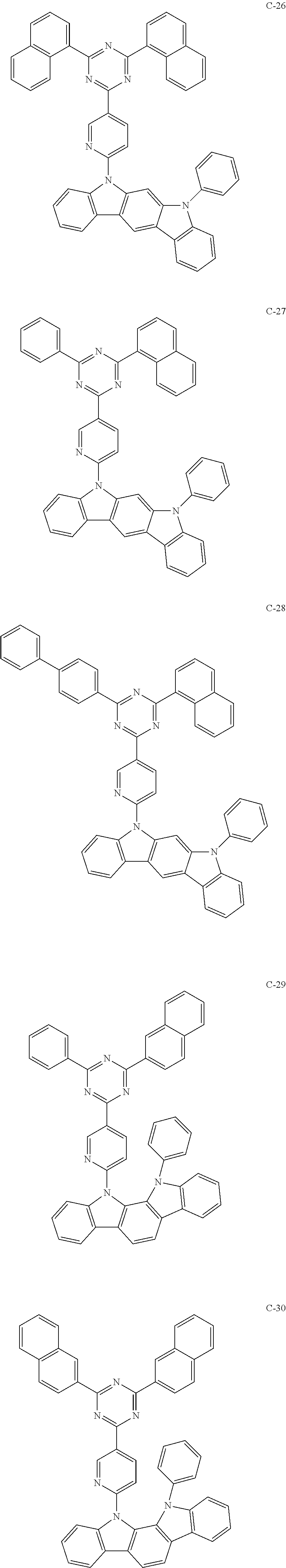 [SMILES: c1ccc(-n2c3ccccc3c3cc4c5ccccc5n(-c5ccc(-c6nc(-c7cccc8ccccc78)nc(-c7cccc8ccccc78)n6)cn5)c4cc32)cc1 ; c1ccc(-c2nc(-c3ccc(-n4c5ccccc5c5cc6c7ccccc7n(-c7ccccc7)c6cc54)nc3)nc(-c3cccc4ccccc34)n2)cc1 ; c1ccc(-c2ccc(-c3nc(-c4ccc(-n5c6ccccc6c6cc7c8ccccc8n(-c8ccccc8)c7cc65)nc4)nc(-c4cccc5ccccc45)n3)cc2)cc1 ; c1ccc(-c2nc(-c3ccc(-n4c5ccccc5c5ccc6c7ccccc7n(-c7ccccc7)c6c54)nc3)nc(-c3ccc4ccccc4c3)n2)cc1 ; c1ccc(-n2c3ccccc3c3ccc4c5ccccc5n(-c5ccc(-c6nc(-c7ccc8ccccc8c7)nc(-c7ccc8ccccc8c7)n6)cn5)c4c32)cc1]

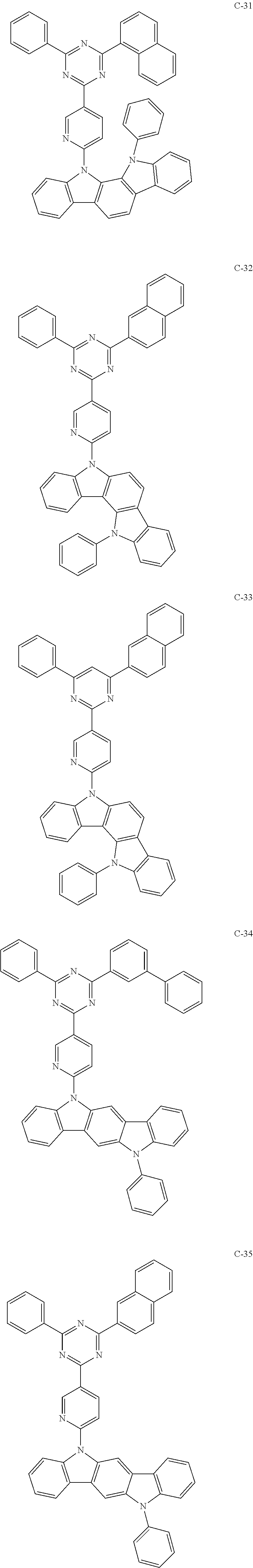 [SMILES: c1ccc(-c2nc(-c3ccc(-n4c5ccccc5c5ccc6c7ccccc7n(-c7ccccc7)c6c54)nc3)nc(-c3cccc4ccccc34)n2)cc1 ; c1ccc(-c2nc(-c3ccc(-n4c5ccccc5c5c4ccc4c6ccccc6n(-c6ccccc6)c45)nc3)nc(-c3ccc4ccccc4c3)n2)cc1 ; c1ccc(-c2cc(-c3ccc4ccccc4c3)nc(-c3ccc(-n4c5ccccc5c5c4ccc4c6ccccc6n(-c6ccccc6)c45)nc3)n2)cc1 ; c1ccc(-c2cccc(-c3nc(-c4ccccc4)nc(-c4ccc(-n5c6ccccc6c6cc7c(cc65)c5ccccc5n7-c5ccccc5)nc4)n3)c2)cc1 ; c1ccc(-c2nc(-c3ccc(-n4c5ccccc5c5cc6c(cc54)c4ccccc4n6-c4ccccc4)nc3)nc(-c3ccc4ccccc4c3)n2)cc1]

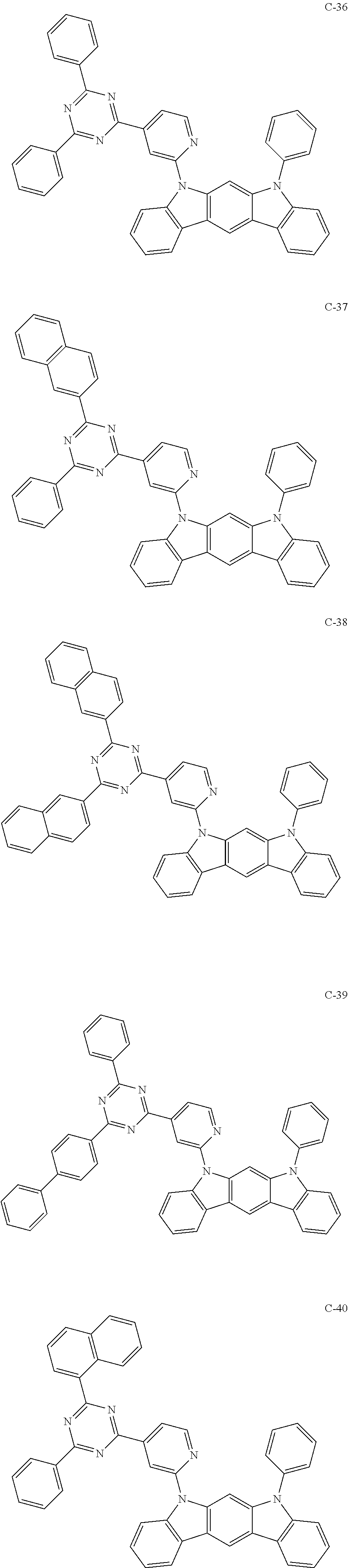 [SMILES: c1ccc(-c2nc(-c3ccccc3)nc(-c3ccnc(-n4c5ccccc5c5cc6c7ccccc7n(-c7ccccc7)c6cc54)c3)n2)cc1 ; c1ccc(-c2nc(-c3ccnc(-n4c5ccccc5c5cc6c7ccccc7n(-c7ccccc7)c6cc54)c3)nc(-c3ccc4ccccc4c3)n2)cc1 ; c1ccc(-n2c3ccccc3c3cc4c5ccccc5n(-c5cc(-c6nc(-c7ccc8ccccc8c7)nc(-c7ccc8ccccc8c7)n6)ccn5)c4cc32)cc1 ; c1ccc(-c2ccc(-c3nc(-c4ccccc4)nc(-c4ccnc(-n5c6ccccc6c6cc7c8ccccc8n(-c8ccccc8)c7cc65)c4)n3)cc2)cc1 ; c1ccc(-c2nc(-c3ccnc(-n4c5ccccc5c5cc6c7ccccc7n(-c7ccccc7)c6cc54)c3)nc(-c3cccc4ccccc34)n2)cc1]

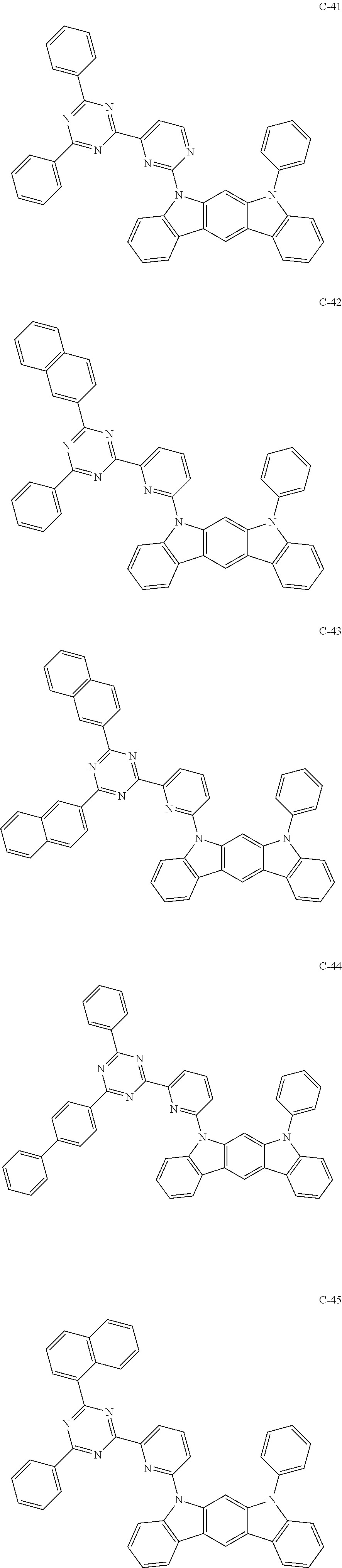 [SMILES: c1ccc(-c2nc(-c3ccccc3)nc(-c3ccnc(-n4c5ccccc5c5cc6c7ccccc7n(-c7ccccc7)c6cc54)n3)n2)cc1 ; c1ccc(-c2nc(-c3ccc4ccccc4c3)nc(-c3cccc(-n4c5ccccc5c5cc6c7ccccc7n(-c7ccccc7)c6cc54)n3)n2)cc1 ; c1ccc(-n2c3ccccc3c3cc4c5ccccc5n(-c5cccc(-c6nc(-c7ccc8ccccc8c7)nc(-c7ccc8ccccc8c7)n6)n5)c4cc32)cc1 ; c1ccc(-c2ccc(-c3nc(-c4ccccc4)nc(-c4cccc(-n5c6ccccc6c6cc7c8ccccc8n(-c8ccccc8)c7cc65)n4)n3)cc2)cc1 ; c1ccc(-c2nc(-c3cccc(-n4c5ccccc5c5cc6c7ccccc7n(-c7ccccc7)c6cc54)n3)nc(-c3cccc4ccccc34)n2)cc1]

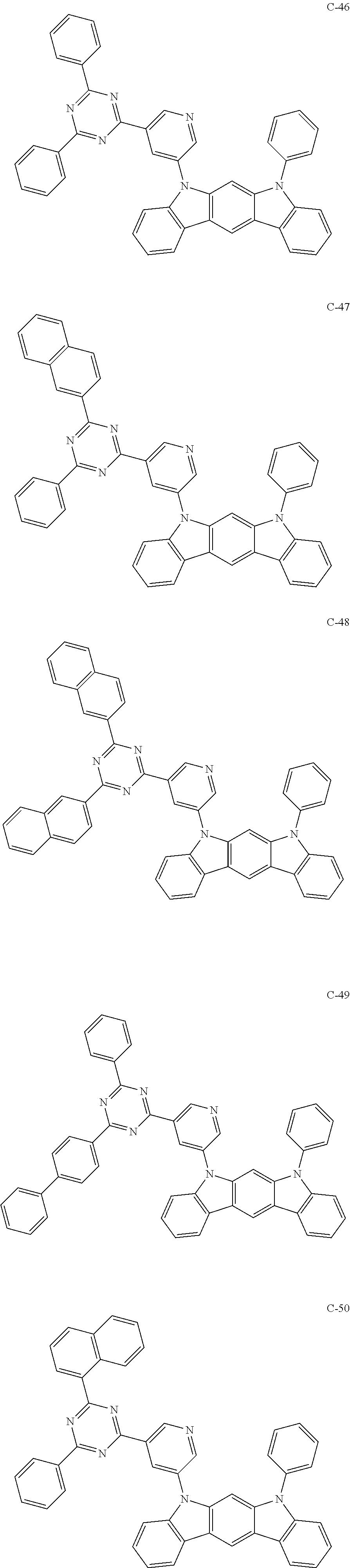 [SMILES: c1ccc(-c2nc(-c3ccccc3)nc(-c3cncc(-n4c5ccccc5c5cc6c7ccccc7n(-c7ccccc7)c6cc54)c3)n2)cc1 ; c1ccc(-c2nc(-c3cncc(-n4c5ccccc5c5cc6c7ccccc7n(-c7ccccc7)c6cc54)c3)nc(-c3ccc4ccccc4c3)n2)cc1 ; c1ccc(-n2c3ccccc3c3cc4c5ccccc5n(-c5cncc(-c6nc(-c7ccc8ccccc8c7)nc(-c7ccc8ccccc8c7)n6)c5)c4cc32)cc1 ; c1ccc(-c2ccc(-c3nc(-c4ccccc4)nc(-c4cncc(-n5c6ccccc6c6cc7c8ccccc8n(-c8ccccc8)c7cc65)c4)n3)cc2)cc1 ; c1ccc(-c2nc(-c3cncc(-n4c5ccccc5c5cc6c7ccccc7n(-c7ccccc7)c6cc54)c3)nc(-c3cccc4ccccc34)n2)cc1]

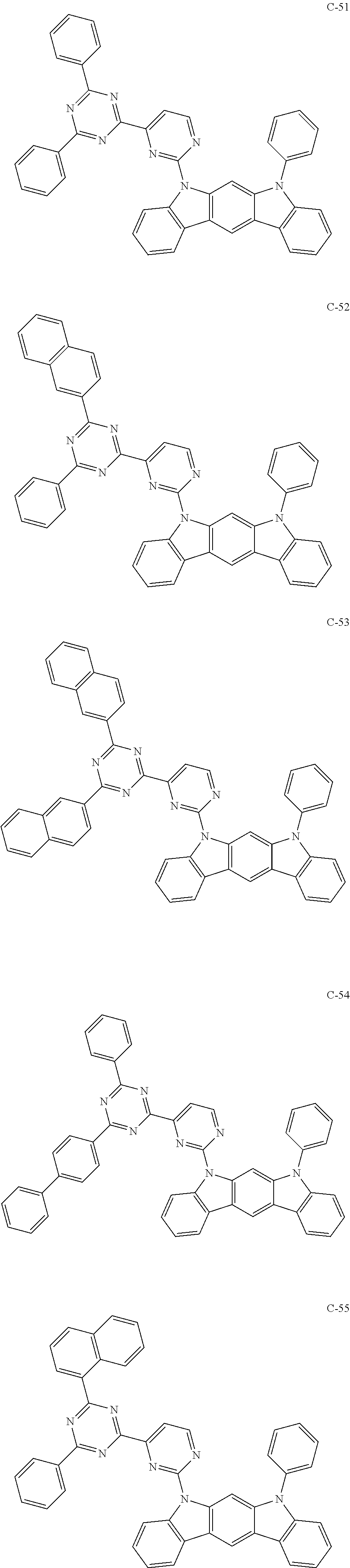 [SMILES: c1ccc(-c2nc(-c3ccccc3)nc(-c3ccnc(-n4c5ccccc5c5cc6c7ccccc7n(-c7ccccc7)c6cc54)n3)n2)cc1 ; c1ccc(-c2nc(-c3ccc4ccccc4c3)nc(-c3ccnc(-n4c5ccccc5c5cc6c7ccccc7n(-c7ccccc7)c6cc54)n3)n2)cc1 ; c1ccc(-n2c3ccccc3c3cc4c5ccccc5n(-c5nccc(-c6nc(-c7ccc8ccccc8c7)nc(-c7ccc8ccccc8c7)n6)n5)c4cc32)cc1 ; c1ccc(-c2ccc(-c3nc(-c4ccccc4)nc(-c4ccnc(-n5c6ccccc6c6cc7c8ccccc8n(-c8ccccc8)c7cc65)n4)n3)cc2)cc1 ; c1ccc(-c2nc(-c3ccnc(-n4c5ccccc5c5cc6c7ccccc7n(-c7ccccc7)c6cc54)n3)nc(-c3cccc4ccccc34)n2)cc1]

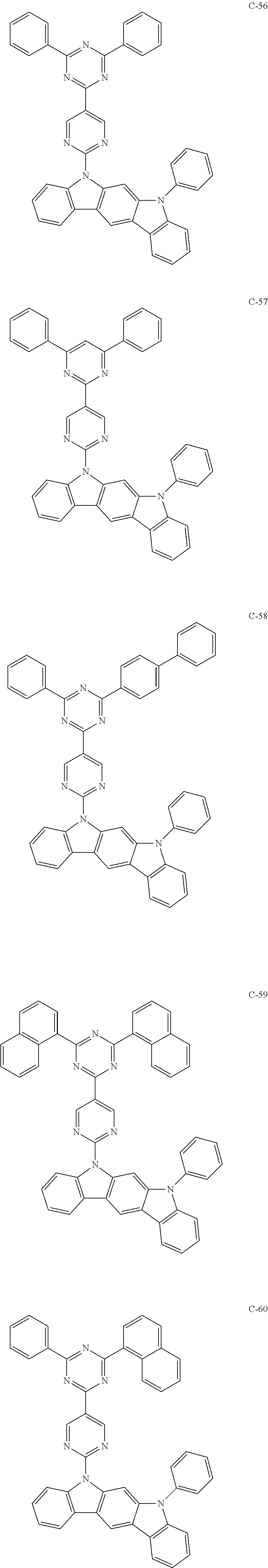 [SMILES: c1ccc(-c2nc(-c3ccccc3)nc(-c3cnc(-n4c5ccccc5c5cc6c7ccccc7n(-c7ccccc7)c6cc54)nc3)n2)cc1 ; c1ccc(-c2cc(-c3ccccc3)nc(-c3cnc(-n4c5ccccc5c5cc6c7ccccc7n(-c7ccccc7)c6cc54)nc3)n2)cc1 ; c1ccc(-c2ccc(-c3nc(-c4ccccc4)nc(-c4cnc(-n5c6ccccc6c6cc7c8ccccc8n(-c8ccccc8)c7cc65)nc4)n3)cc2)cc1 ; c1ccc(-n2c3ccccc3c3cc4c5ccccc5n(-c5ncc(-c6nc(-c7cccc8ccccc78)nc(-c7cccc8ccccc78)n6)cn5)c4cc32)cc1 ; c1ccc(-c2nc(-c3cnc(-n4c5ccccc5c5cc6c7ccccc7n(-c7ccccc7)c6cc54)nc3)nc(-c3cccc4ccccc34)n2)cc1]

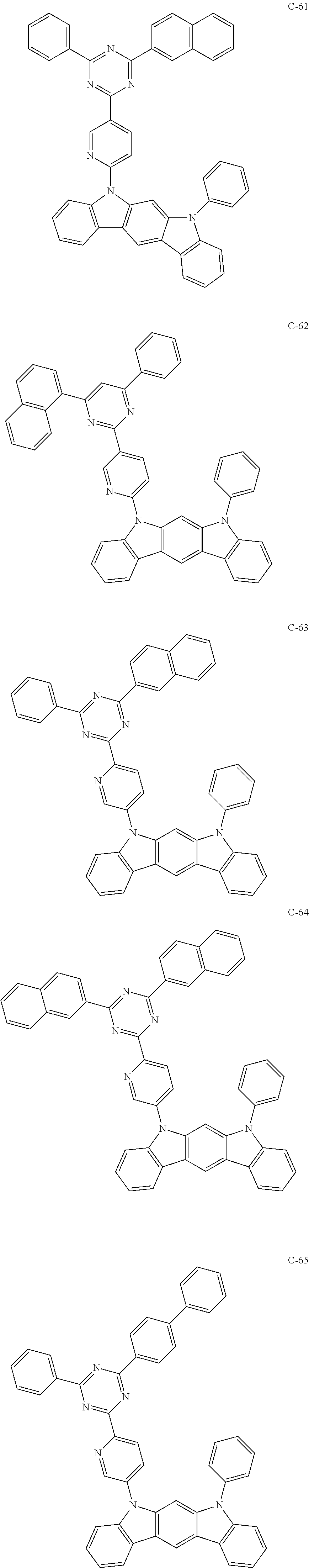 [SMILES: c1ccc(-c2nc(-c3ccc(-n4c5ccccc5c5cc6c7ccccc7n(-c7ccccc7)c6cc54)nc3)nc(-c3ccc4ccccc4c3)n2)cc1 ; c1ccc(-c2cc(-c3cccc4ccccc34)nc(-c3ccc(-n4c5ccccc5c5cc6c7ccccc7n(-c7ccccc7)c6cc54)nc3)n2)cc1 ; c1ccc(-c2nc(-c3ccc4ccccc4c3)nc(-c3ccc(-n4c5ccccc5c5cc6c7ccccc7n(-c7ccccc7)c6cc54)cn3)n2)cc1 ; c1ccc(-n2c3ccccc3c3cc4c5ccccc5n(-c5ccc(-c6nc(-c7ccc8ccccc8c7)nc(-c7ccc8ccccc8c7)n6)nc5)c4cc32)cc1 ; c1ccc(-c2ccc(-c3nc(-c4ccccc4)nc(-c4ccc(-n5c6ccccc6c6cc7c8ccccc8n(-c8ccccc8)c7cc65)cn4)n3)cc2)cc1]

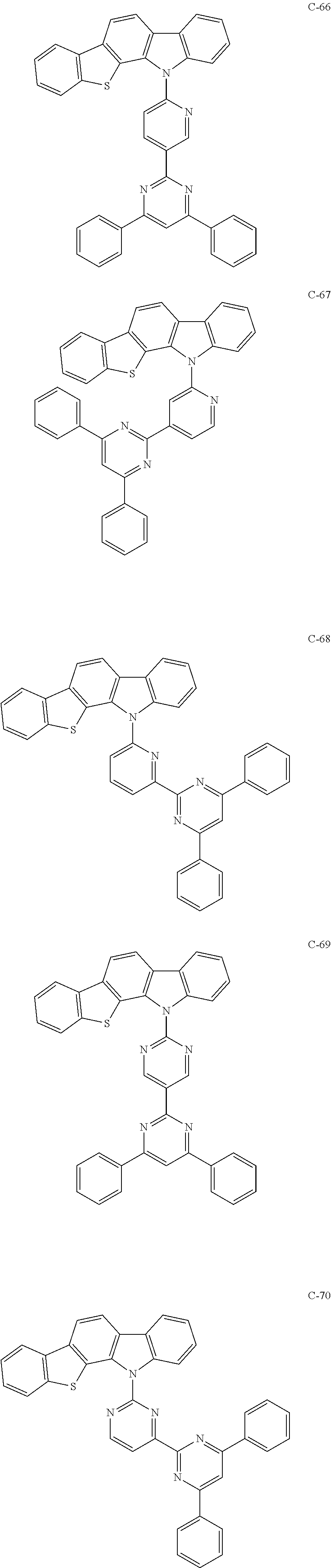 [SMILES: c1ccc(-c2cc(-c3ccccc3)nc(-c3ccc(-n4c5ccccc5c5ccc6c7ccccc7sc6c54)nc3)n2)cc1 ; c1ccc(-c2cc(-c3ccccc3)nc(-c3ccnc(-n4c5ccccc5c5ccc6c7ccccc7sc6c54)c3)n2)cc1 ; c1ccc(-c2cc(-c3ccccc3)nc(-c3cccc(-n4c5ccccc5c5ccc6c7ccccc7sc6c54)n3)n2)cc1 ; c1ccc(-c2cc(-c3ccccc3)nc(-c3cnc(-n4c5ccccc5c5ccc6c7ccccc7sc6c54)nc3)n2)cc1 ; c1ccc(-c2cc(-c3ccccc3)nc(-c3ccnc(-n4c5ccccc5c5ccc6c7ccccc7sc6c54)n3)n2)cc1]

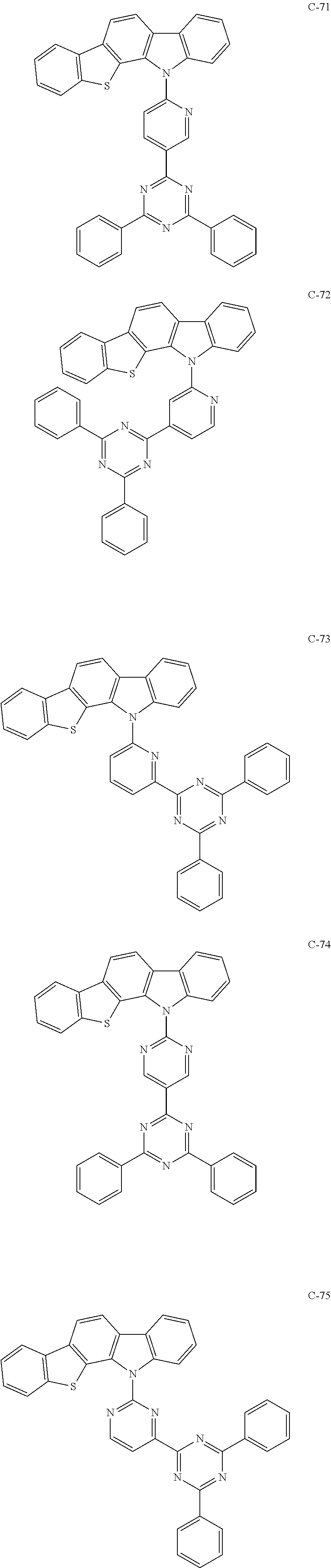 [SMILES: c1ccc(-c2nc(-c3ccccc3)nc(-c3ccc(-n4c5ccccc5c5ccc6c7ccccc7sc6c54)nc3)n2)cc1 ; c1ccc(-c2nc(-c3ccccc3)nc(-c3ccnc(-n4c5ccccc5c5ccc6c7ccccc7sc6c54)c3)n2)cc1 ; c1ccc(-c2nc(-c3ccccc3)nc(-c3cccc(-n4c5ccccc5c5ccc6c7ccccc7sc6c54)n3)n2)cc1 ; c1ccc(-c2nc(-c3ccccc3)nc(-c3cnc(-n4c5ccccc5c5ccc6c7ccccc7sc6c54)nc3)n2)cc1 ; c1ccc(-c2nc(-c3ccccc3)nc(-c3ccnc(-n4c5ccccc5c5ccc6c7ccccc7sc6c54)n3)n2)cc1]

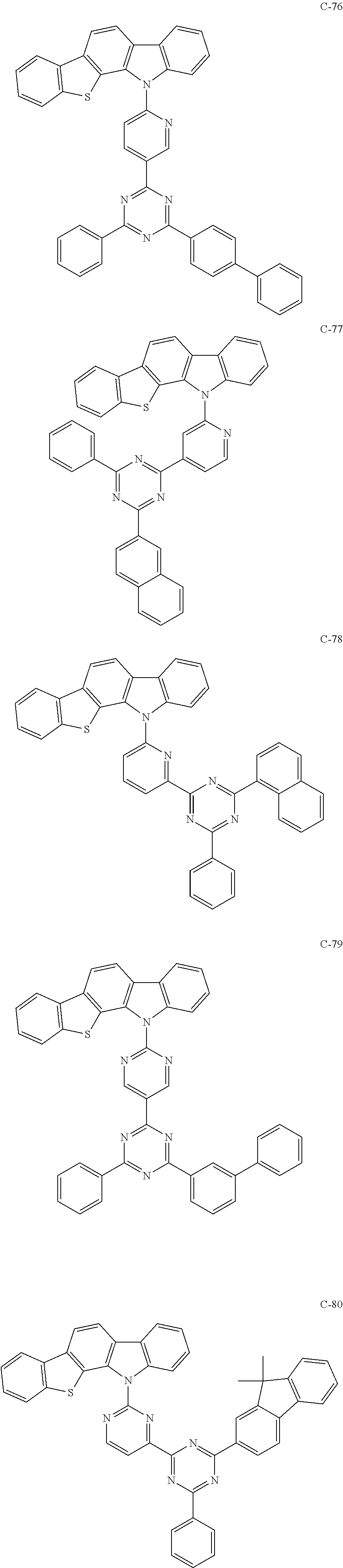

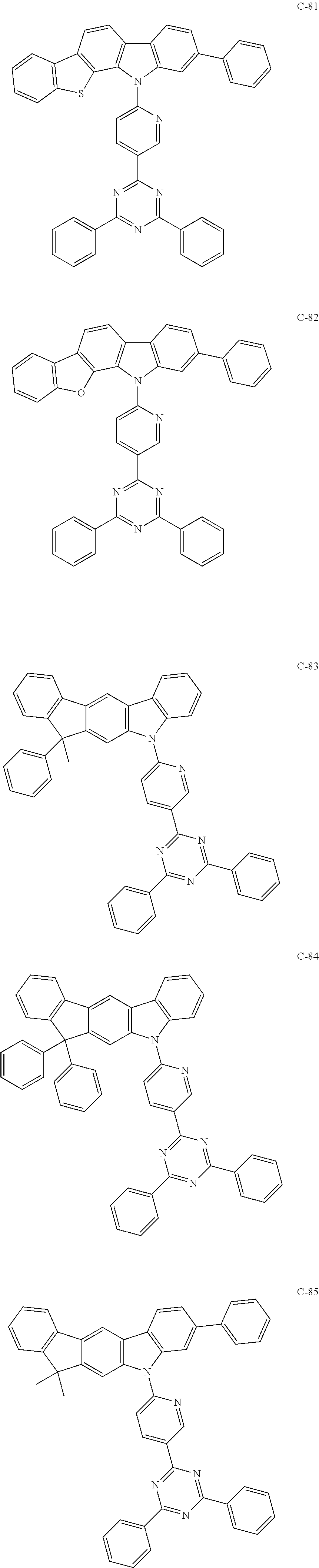 [SMILES: c1ccc(-c2ccc3c4ccc5c6ccccc6sc5c4n(-c4ccc(-c5nc(-c6ccccc6)nc(-c6ccccc6)n5)cn4)c3c2)cc1 ; c1ccc(-c2ccc3c4ccc5c6ccccc6oc5c4n(-c4ccc(-c5nc(-c6ccccc6)nc(-c6ccccc6)n5)cn4)c3c2)cc1 ; CC1(c2ccccc2)c2ccccc2-c2cc3c4ccccc4n(-c4ccc(-c5nc(-c6ccccc6)nc(-c6ccccc6)n5)cn4)c3cc21 ; c1ccc(-c2nc(-c3ccccc3)nc(-c3ccc(-n4c5ccccc5c5cc6c(cc54)C(c4ccccc4)(c4ccccc4)c4ccccc4-6)nc3)n2)cc1 ; CC1(C)c2ccccc2-c2cc3c4ccc(-c5ccccc5)cc4n(-c4ccc(-c5nc(-c6ccccc6)nc(-c6ccccc6)n5)cn4)c3cc21]

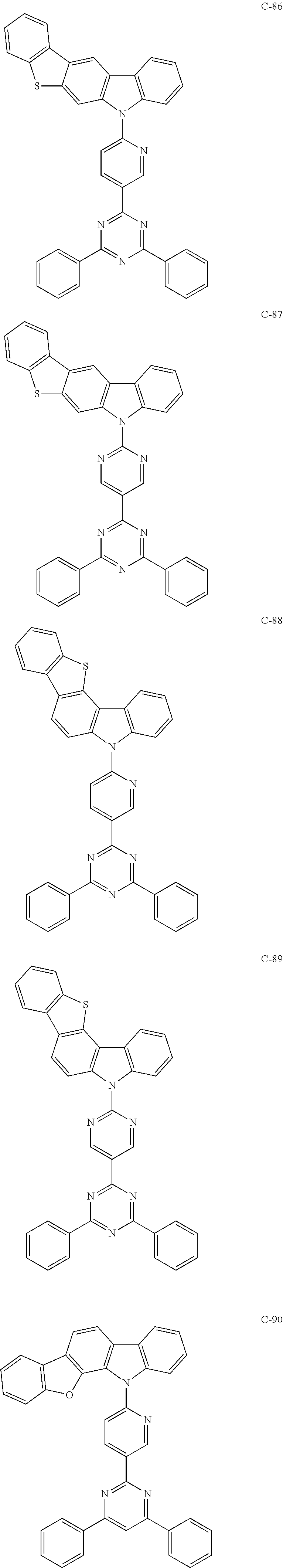

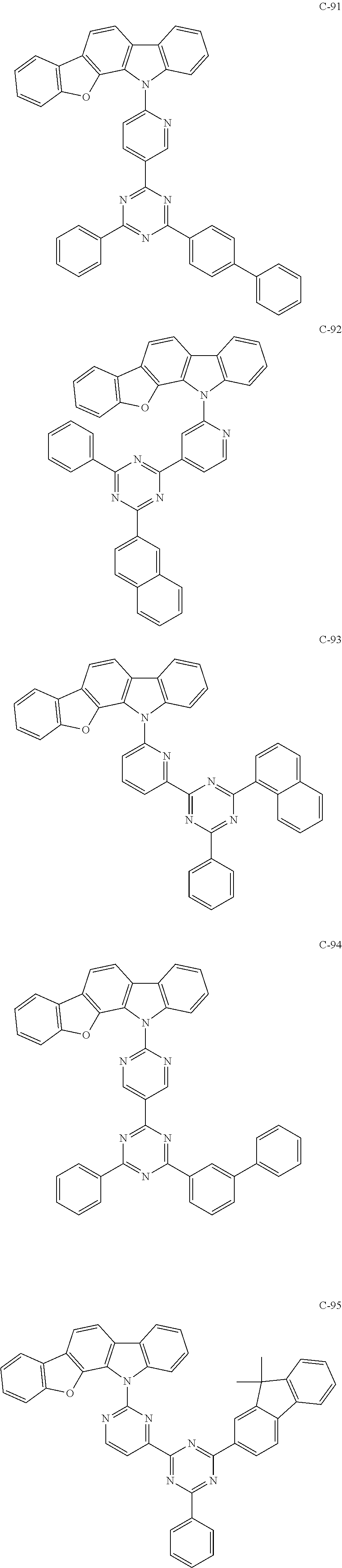

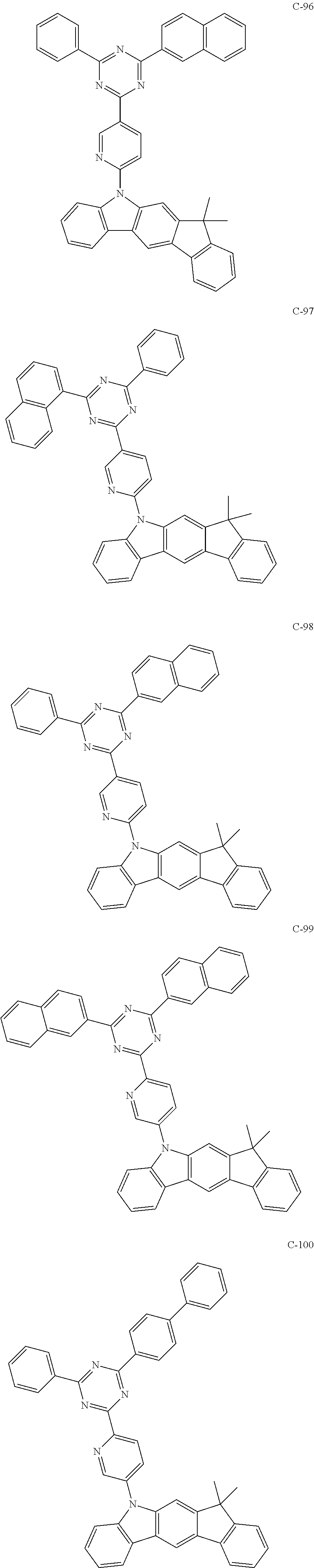 [SMILES: CC1(C)c2ccccc2-c2cc3c4ccccc4n(-c4ccc(-c5nc(-c6ccccc6)nc(-c6ccc7ccccc7c6)n5)cn4)c3cc21 ; CC1(C)c2ccccc2-c2cc3c4ccccc4n(-c4ccc(-c5nc(-c6ccccc6)nc(-c6cccc7ccccc67)n5)cn4)c3cc21 ; CC1(C)c2ccccc2-c2cc3c4ccccc4n(-c4ccc(-c5nc(-c6ccccc6)nc(-c6ccc7ccccc7c6)n5)cn4)c3cc21 ; CC1(C)c2ccccc2-c2cc3c4ccccc4n(-c4ccc(-c5nc(-c6ccc7ccccc7c6)nc(-c6ccc7ccccc7c6)n5)nc4)c3cc21 ; CC1(C)c2ccccc2-c2cc3c4ccccc4n(-c4ccc(-c5nc(-c6ccccc6)nc(-c6ccc(-c7ccccc7)cc6)n5)nc4)c3cc21]

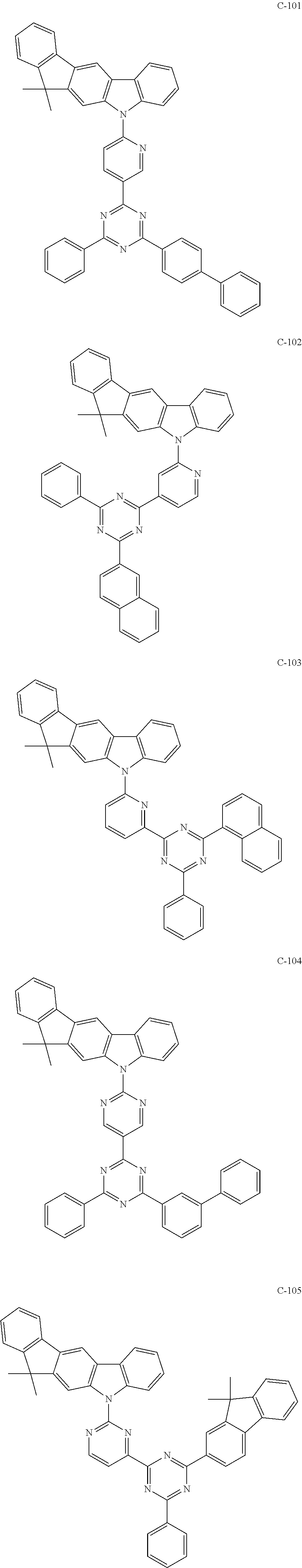 [SMILES: CC1(C)c2ccccc2-c2cc3c4ccccc4n(-c4ccc(-c5nc(-c6ccccc6)nc(-c6ccc(-c7ccccc7)cc6)n5)cn4)c3cc21 ; CC1(C)c2ccccc2-c2cc3c4ccccc4n(-c4cc(-c5nc(-c6ccccc6)nc(-c6ccc7ccccc7c6)n5)ccn4)c3cc21 ; CC1(C)c2ccccc2-c2cc3c4ccccc4n(-c4cccc(-c5nc(-c6ccccc6)nc(-c6cccc7ccccc67)n5)n4)c3cc21 ; CC1(C)c2ccccc2-c2cc3c4ccccc4n(-c4ncc(-c5nc(-c6ccccc6)nc(-c6cccc(-c7ccccc7)c6)n5)cn4)c3cc21 ; CC1(C)c2ccccc2-c2ccc(-c3nc(-c4ccccc4)nc(-c4ccnc(-n5c6ccccc6c6cc7c(cc65)C(C)(C)c5ccccc5-7)n4)n3)cc21]

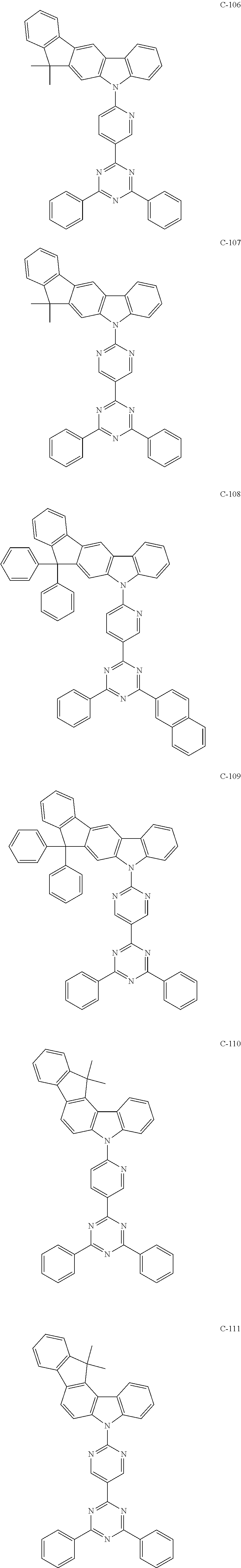

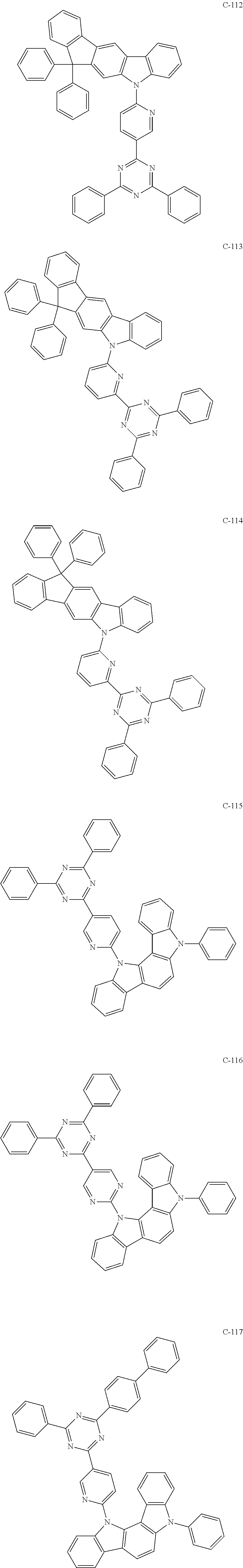

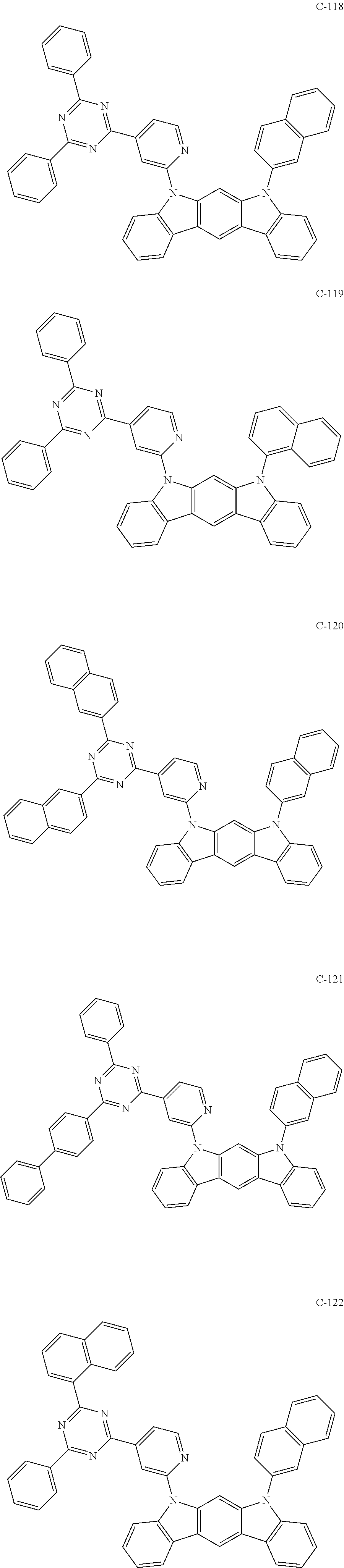 [SMILES: c1ccc(-c2nc(-c3ccccc3)nc(-c3ccnc(-n4c5ccccc5c5cc6c7ccccc7n(-c7ccc8ccccc8c7)c6cc54)c3)n2)cc1 ; c1ccc(-c2nc(-c3ccccc3)nc(-c3ccnc(-n4c5ccccc5c5cc6c7ccccc7n(-c7cccc8ccccc78)c6cc54)c3)n2)cc1 ; c1ccc2cc(-c3nc(-c4ccnc(-n5c6ccccc6c6cc7c8ccccc8n(-c8ccc9ccccc9c8)c7cc65)c4)nc(-c4ccc5ccccc5c4)n3)ccc2c1 ; c1ccc(-c2ccc(-c3nc(-c4ccccc4)nc(-c4ccnc(-n5c6ccccc6c6cc7c8ccccc8n(-c8ccc9ccccc9c8)c7cc65)c4)n3)cc2)cc1 ; c1ccc(-c2nc(-c3ccnc(-n4c5ccccc5c5cc6c7ccccc7n(-c7ccc8ccccc8c7)c6cc54)c3)nc(-c3cccc4ccccc34)n2)cc1]

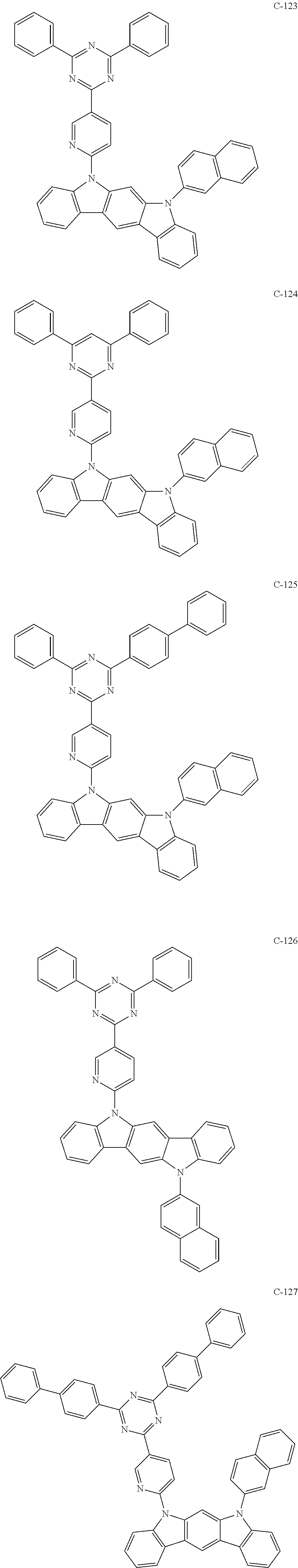 [SMILES: c1ccc(-c2nc(-c3ccccc3)nc(-c3ccc(-n4c5ccccc5c5cc6c7ccccc7n(-c7ccc8ccccc8c7)c6cc54)nc3)n2)cc1 ; c1ccc(-c2cc(-c3ccccc3)nc(-c3ccc(-n4c5ccccc5c5cc6c7ccccc7n(-c7ccc8ccccc8c7)c6cc54)nc3)n2)cc1 ; c1ccc(-c2ccc(-c3nc(-c4ccccc4)nc(-c4ccc(-n5c6ccccc6c6cc7c8ccccc8n(-c8ccc9ccccc9c8)c7cc65)nc4)n3)cc2)cc1 ; c1ccc(-c2nc(-c3ccccc3)nc(-c3ccc(-n4c5ccccc5c5cc6c(cc54)c4ccccc4n6-c4ccc5ccccc5c4)nc3)n2)cc1 ; c1ccc(-c2ccc(-c3nc(-c4ccc(-c5ccccc5)cc4)nc(-c4ccc(-n5c6ccccc6c6cc7c8ccccc8n(-c8ccc9ccccc9c8)c7cc65)nc4)n3)cc2)cc1]

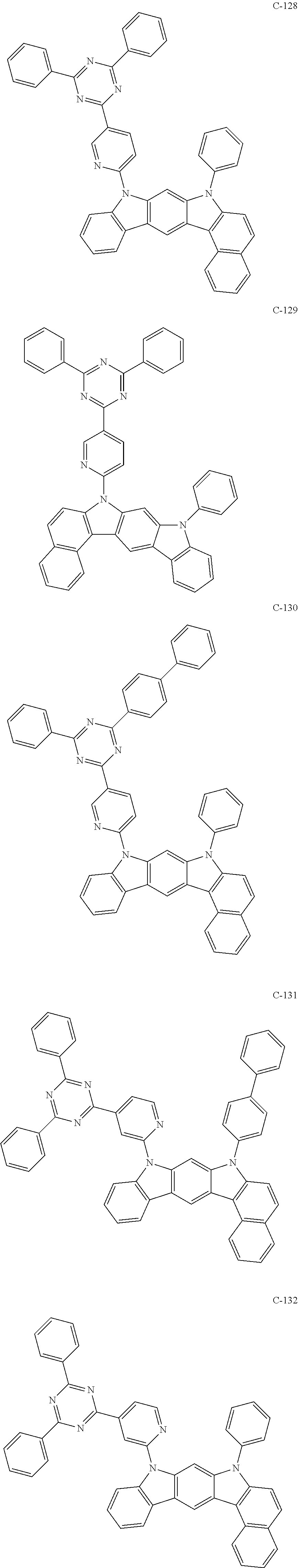 [SMILES: c1ccc(-c2nc(-c3ccccc3)nc(-c3ccc(-n4c5ccccc5c5cc6c7c8ccccc8ccc7n(-c7ccccc7)c6cc54)nc3)n2)cc1 ; c1ccc(-c2nc(-c3ccccc3)nc(-c3ccc(-n4c5cc6c(cc5c5c7ccccc7ccc54)c4ccccc4n6-c4ccccc4)nc3)n2)cc1 ; c1ccc(-c2ccc(-c3nc(-c4ccccc4)nc(-c4ccc(-n5c6ccccc6c6cc7c8c9ccccc9ccc8n(-c8ccccc8)c7cc65)nc4)n3)cc2)cc1 ; c1ccc(-c2ccc(-n3c4cc5c(cc4c4c6ccccc6ccc43)c3ccccc3n5-c3cc(-c4nc(-c5ccccc5)nc(-c5ccccc5)n4)ccn3)cc2)cc1 ; c1ccc(-c2nc(-c3ccccc3)nc(-c3ccnc(-n4c5ccccc5c5cc6c7c8ccccc8ccc7n(-c7ccccc7)c6cc54)c3)n2)cc1]

The compound of formula 1 according to the present invention can be prepared by a synthetic method known to a person skilled in the art. For example, it can be prepared according to the following reaction scheme.

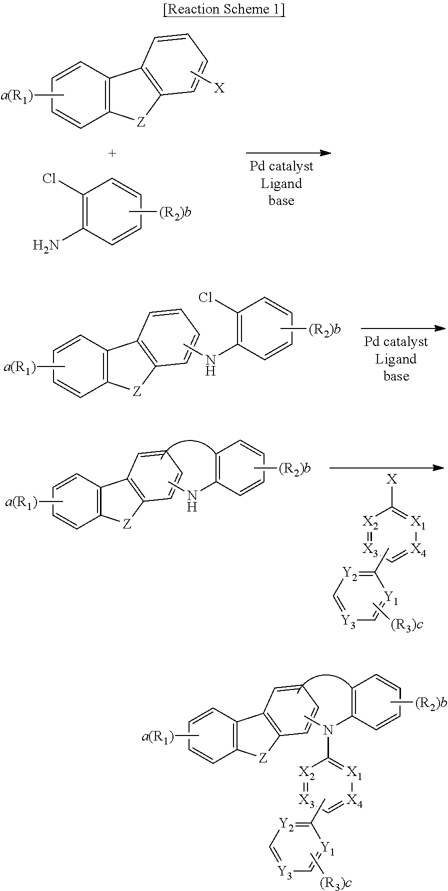

wherein R₁ to R₃, Z, X₁ to X₄, Y₁ to Y₃, and a to c are as defined in formula 1, and X represents halogen.

The present invention provides a phosphorous host material comprising the compound of formula 1, and an organic electroluminescent device comprising the material.

The above material can be comprised of the compound of formula 1 alone, or can further include conventional materials generally used in phosphorous host materials.

The organic electroluminescent device comprises a first electrode; a second electrode; and at least one organic layer between the first and second electrodes. The organic layer may comprise the phosphorous host material of the present invention.

One of the first and second electrodes can be an anode, and the other can be a cathode. The organic layer comprises a light-emitting layer, and may further comprise at least one layer selected from the group consisting of a hole injection layer, a hole transport layer, an electron transport layer, an electron injection layer, an interlayer, a hole blocking layer, and an electron blocking layer.

The phosphorous host material of the present invention can be comprised in the light-emitting layer. Preferably, the light-emitting layer can further comprise one or more dopants. In the phosphorous host material of the present invention, another compound can be comprised as a second host material besides the compound of formula 1 (first host material). Herein, the weight ratio of the first host material to the second host material is in the range of 1:99 to 99:1.

The second host material can be any of the known phosphorescent hosts. Specifically, the compound selected from the group consisting of the compounds of formulas 11 to 16 below is preferable in terms of luminous efficiency.

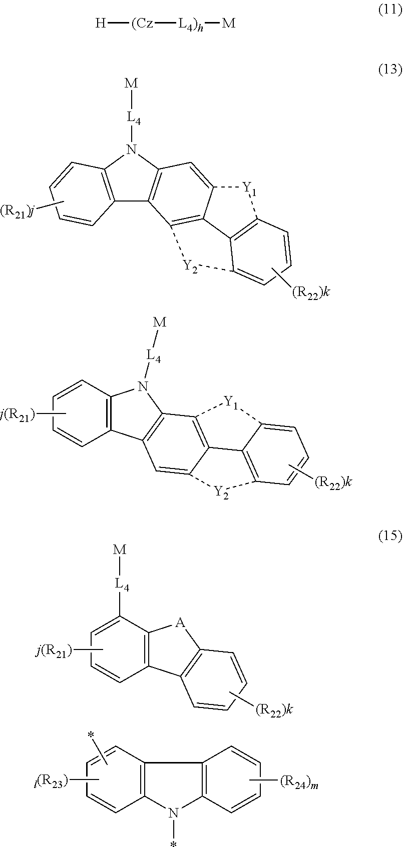

A represents —O— or —S—;
R₂₁ to R₂₄ each independently represent hydrogen, deuterium, a halogen, a substituted or unsubstituted (C1-C30)alkyl, a substituted of unsubstituted (C6-C30)aryl, a substituted or unsubstituted 5- to 30-membered heteroaryl, or —SiR₂₅R₂₆R₂₇, R₂₅ to R₂₇ each independently represent a substituted or unsubstituted (C1-C30)alkyl, or a substituted or unsubstituted (C6-C30)aryl; L₄ represents a single bond, a substituted or unsubstituted (C6-C30)arylene, or a substituted or unsubstituted 5- to 30-membered heteroarylene; M represents a substituted or unsubstituted (C6-C30)aryl, or a substituted or unsubstituted 5- to 30-membered heteroaryl; Y₁ and Y₂ each independently represent —O—, —S—, —N(R₃₁)—, or —C(R₃₂)(R₃₃)—, provided that Y₁ and Y₂ do not simultaneously exist; R₃₁ to R₃₃ each independently represent a substituted or unsubstituted (C1-C30)alkyl, a substituted or unsubstituted (C6-C30)aryl, or a substituted or unsubstituted 5- to 30-membered heteroaryl, and R₃₂ and R₃₃ may be the same or different; h and i each independently represent an integer of 1 to 3; j, k, l, and m each independently represent an integer of 0 to 4; and where h, i, j, k, l, or m is an integer of 2 or more, each of (Cz-L₄), each of (Cz), each of R₂₁, each of R₂₂, each of R₂₃, or each of R₂₄ may be the same or different.

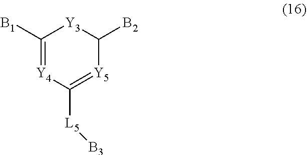

wherein
Y₃ to Y₅ each independently represent CR₃₄ or N;
R₃₄ represents hydrogen, a substituted or unsubstituted (C1-C30)alkyl, a substituted of unsubstituted (C6-C30)aryl, or a substituted or unsubstituted 5- to 30-membered heteroaryl;
B₁ and B₂ each independently represent hydrogen, a substituted of unsubstituted (C6-C30)aryl, or a substituted or unsubstituted 5- to 30-membered heteroaryl;
B₃ represents a substituted of unsubstituted (C6-C30)aryl, or a substituted or unsubstituted 5- to 30-membered heteroaryl; and
L₅ represents a single bond, a substituted of unsubstituted (C6-C30)arylene, or a substituted or unsubstituted 5- to 30-membered heteroarylene.

Specifically, preferable examples of the second host material are as follows:

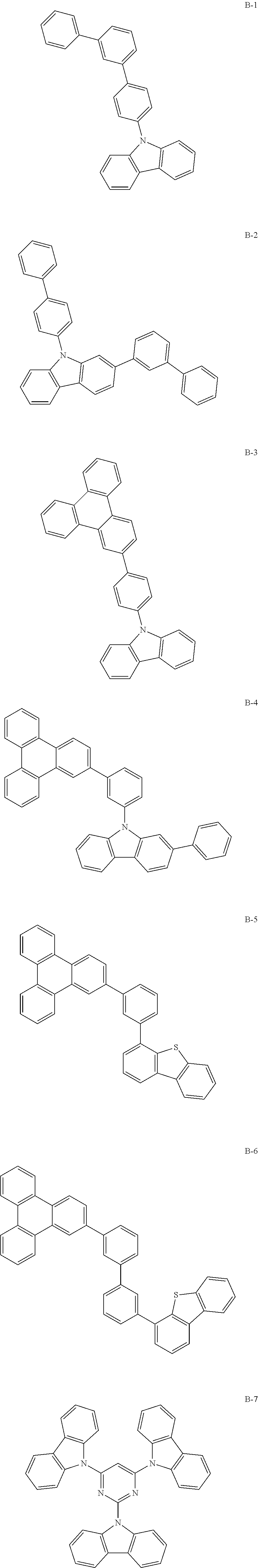

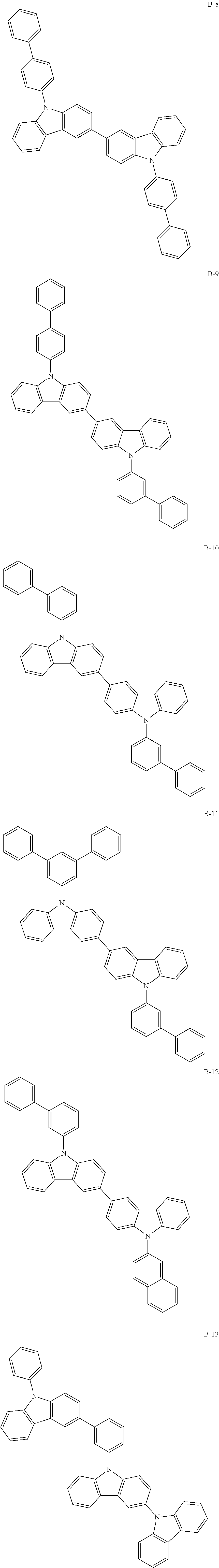

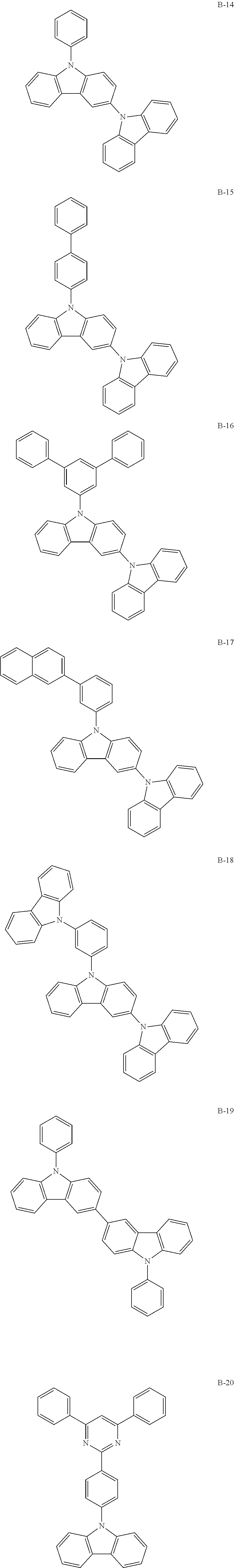

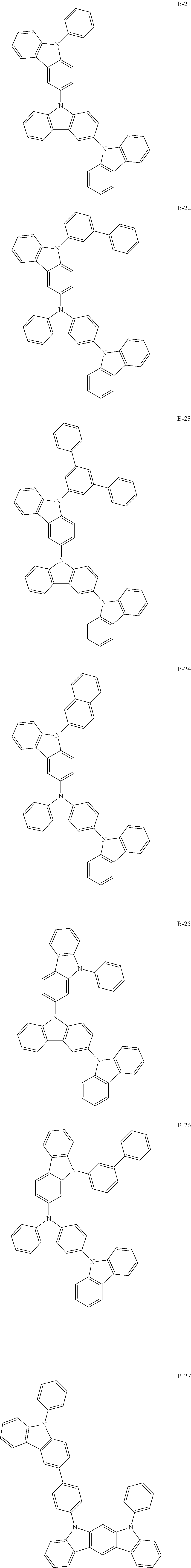

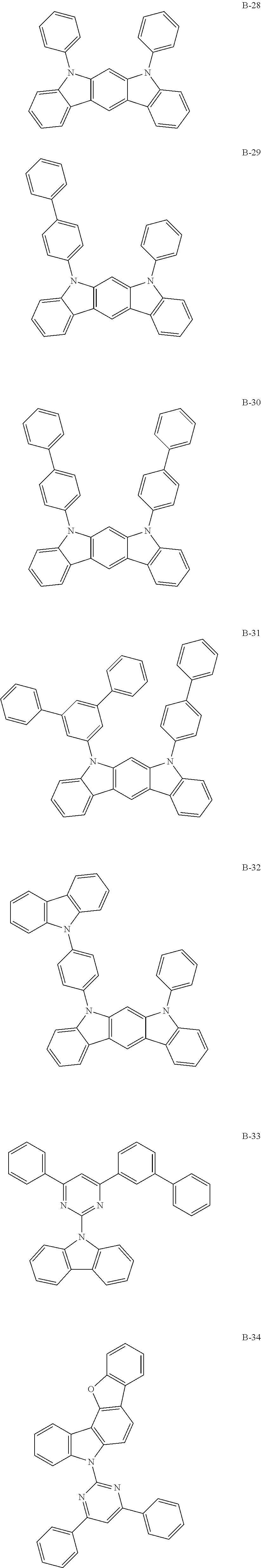

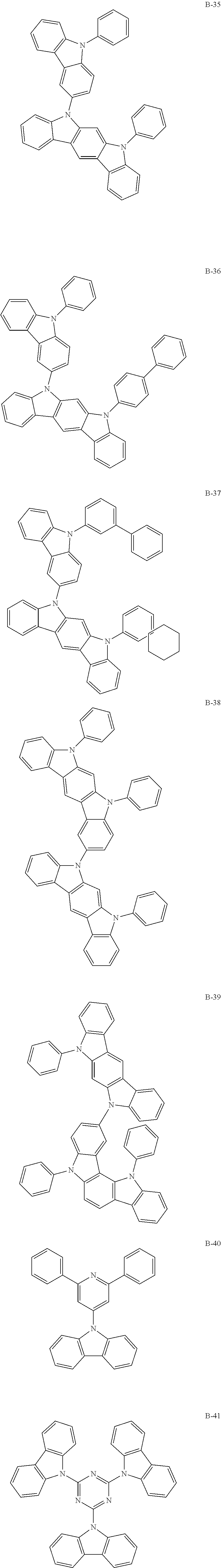

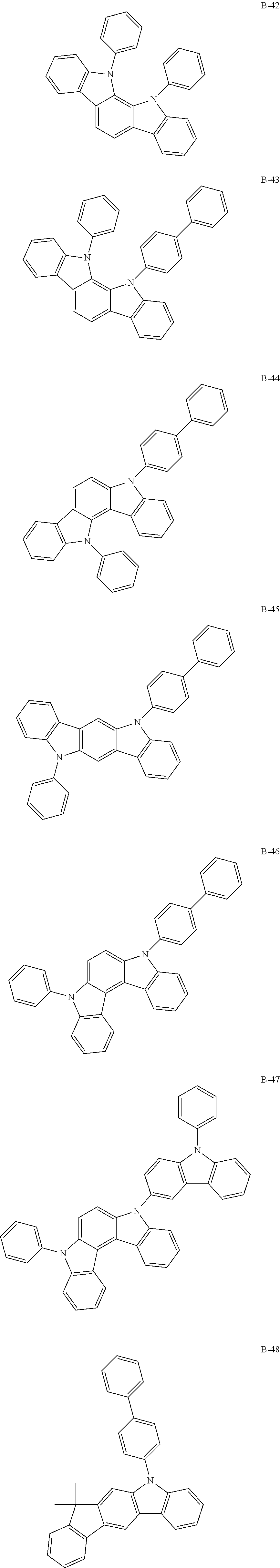

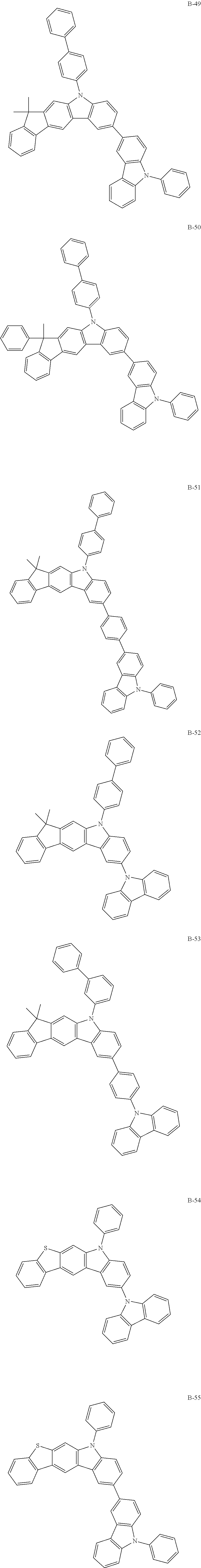

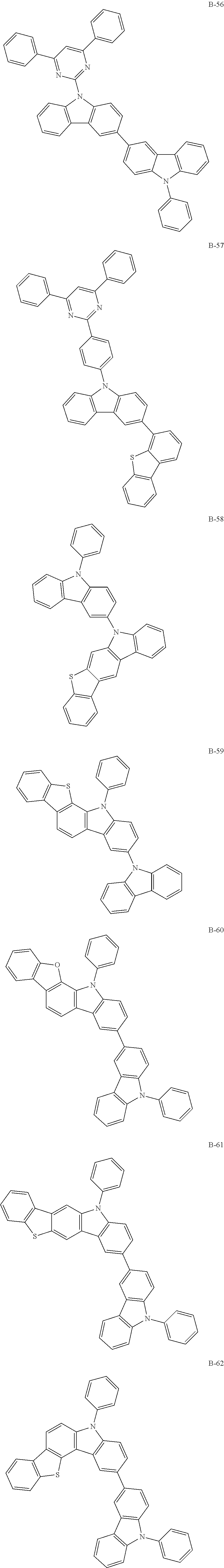

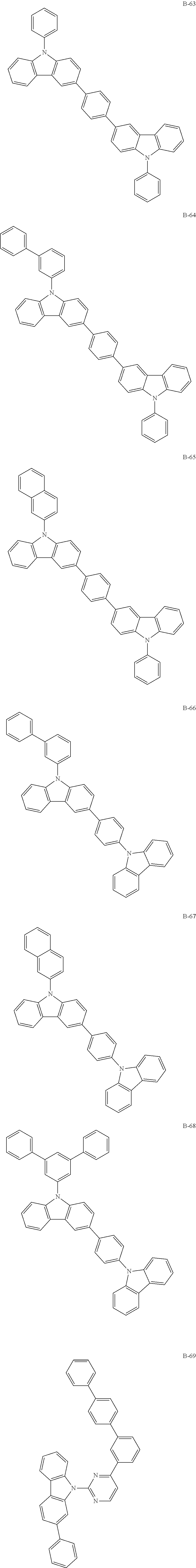

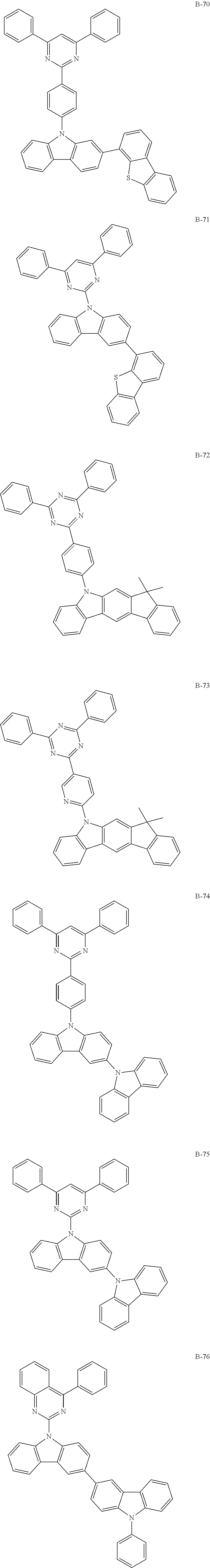

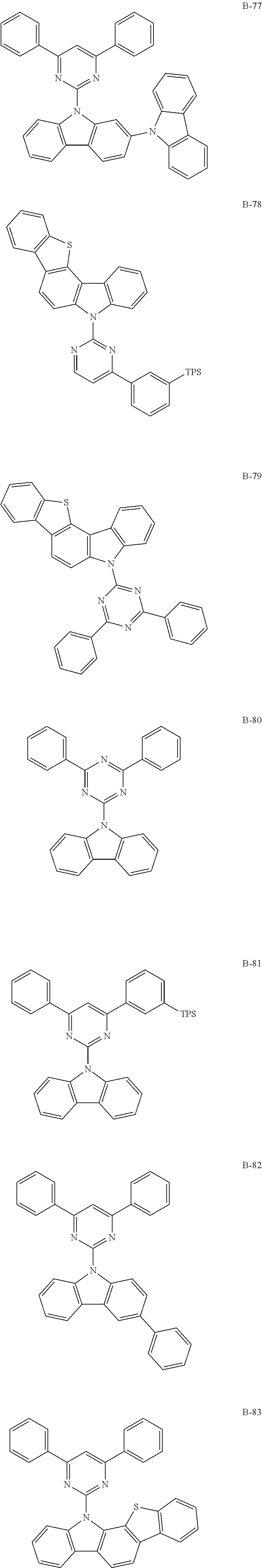

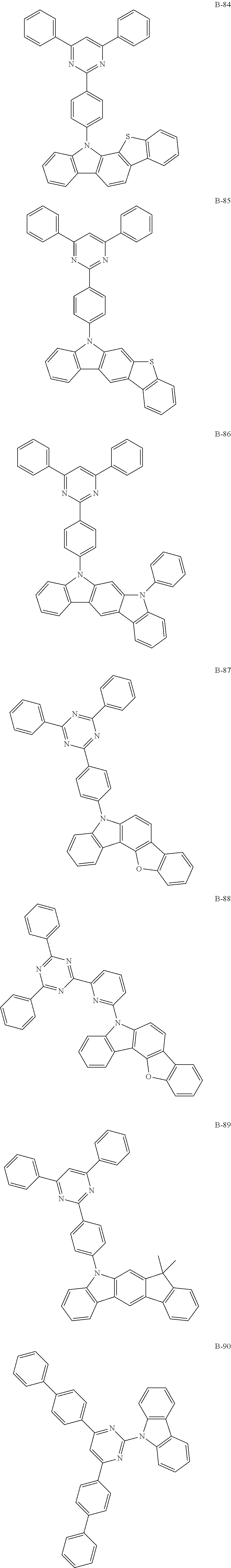

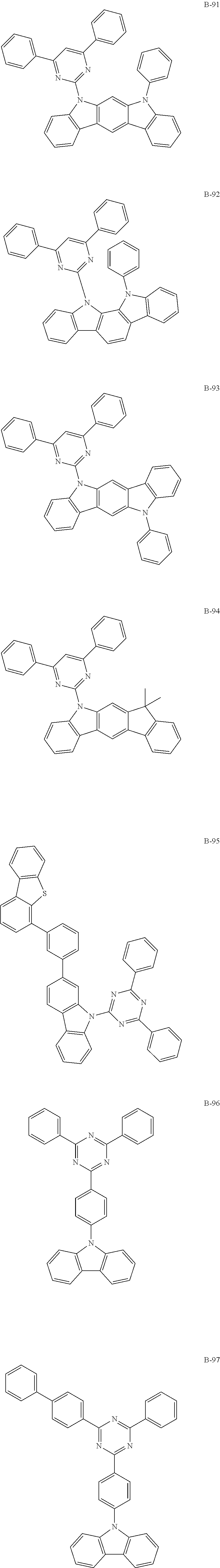

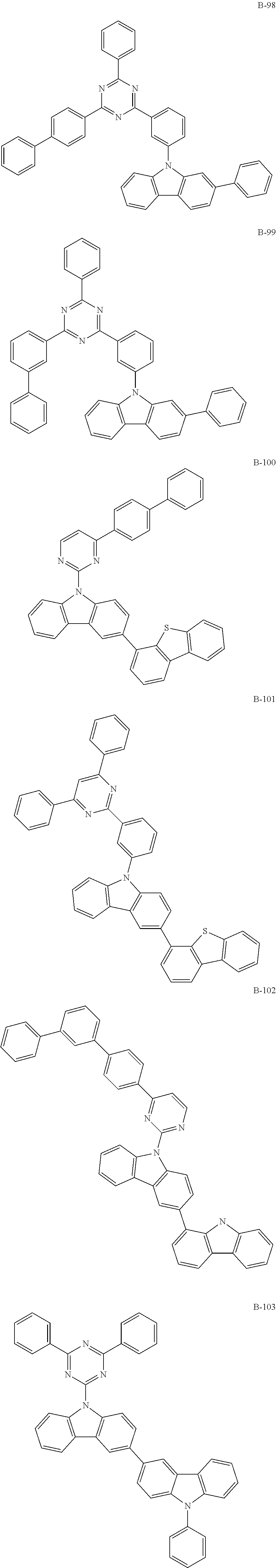

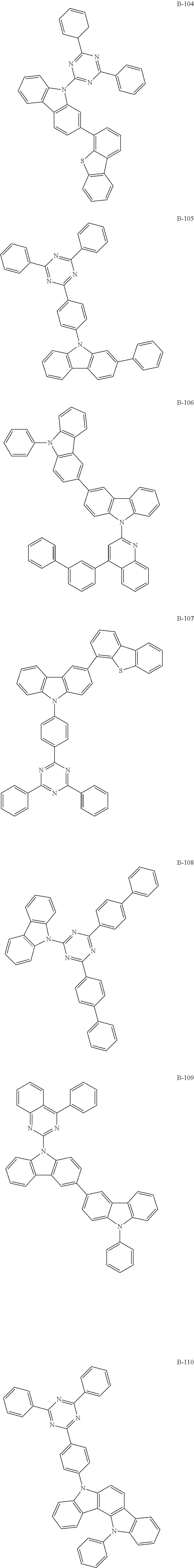

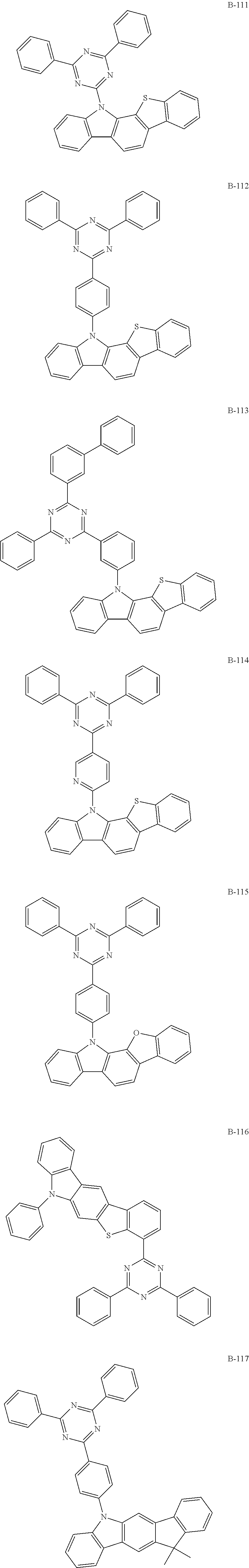

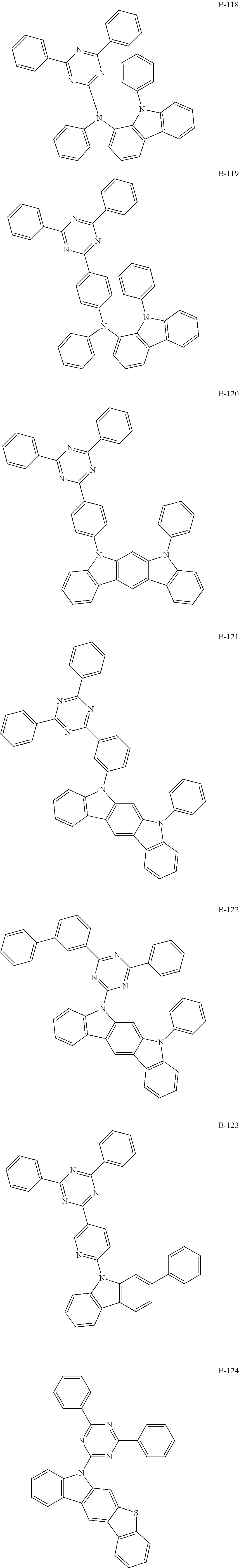

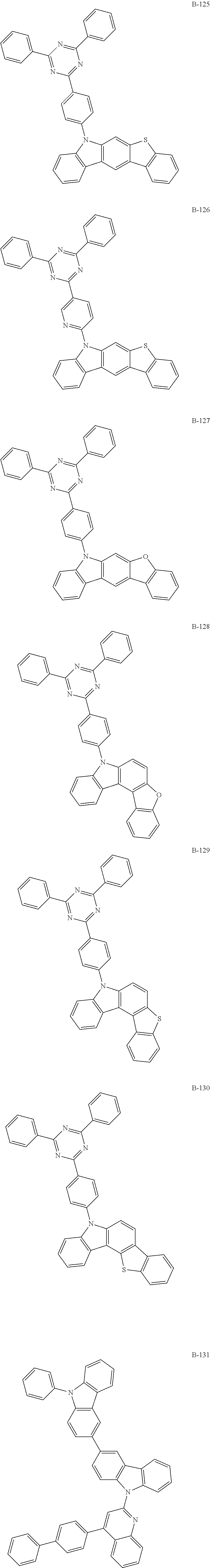

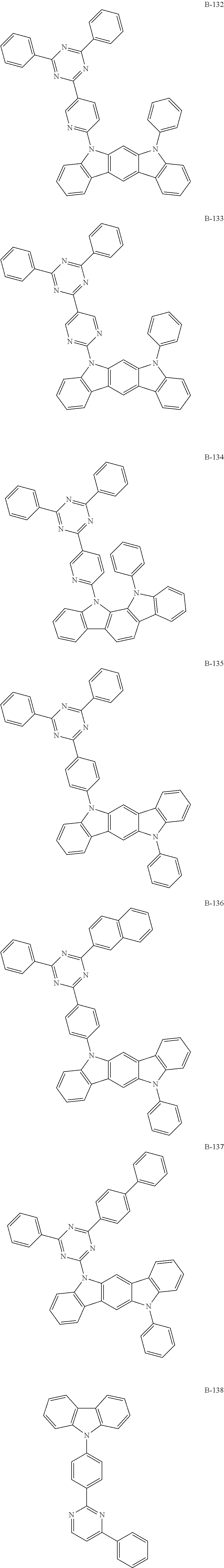

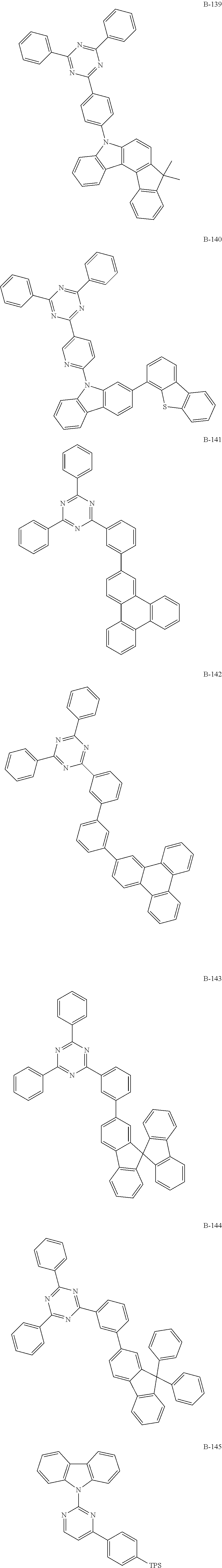

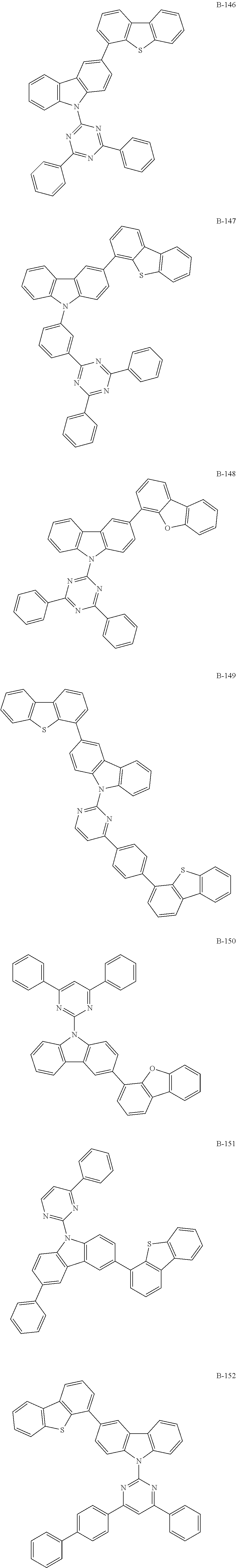

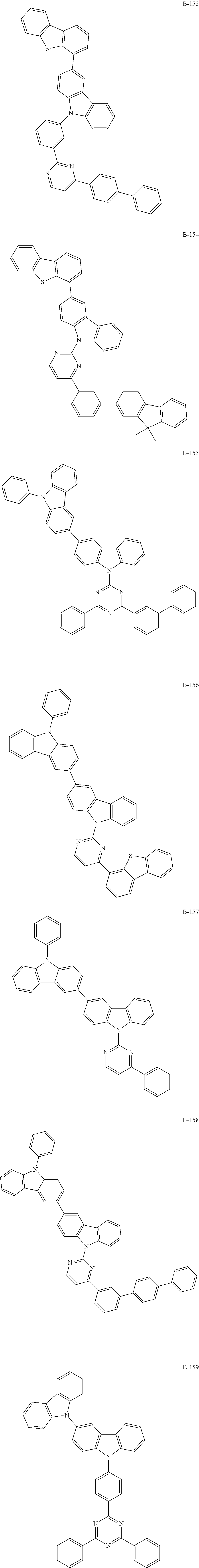

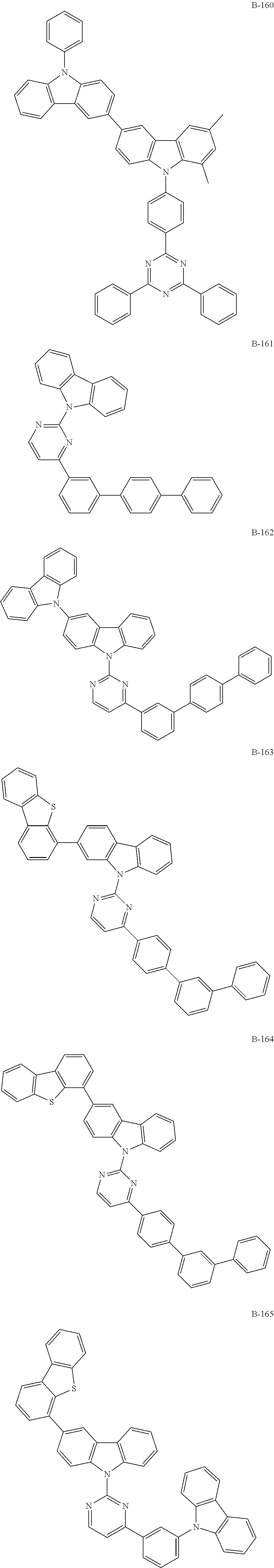

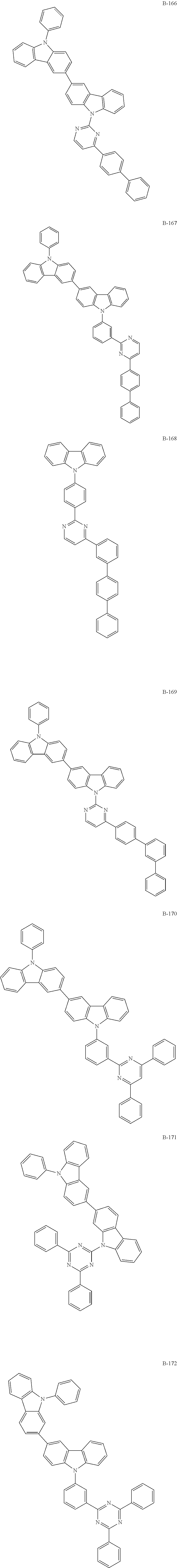

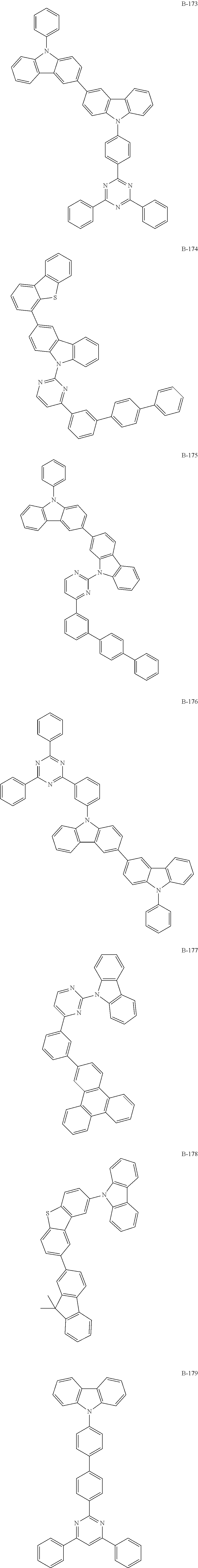

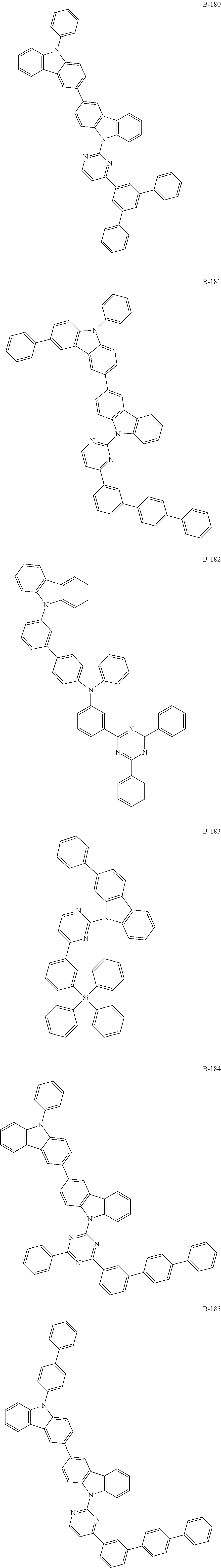

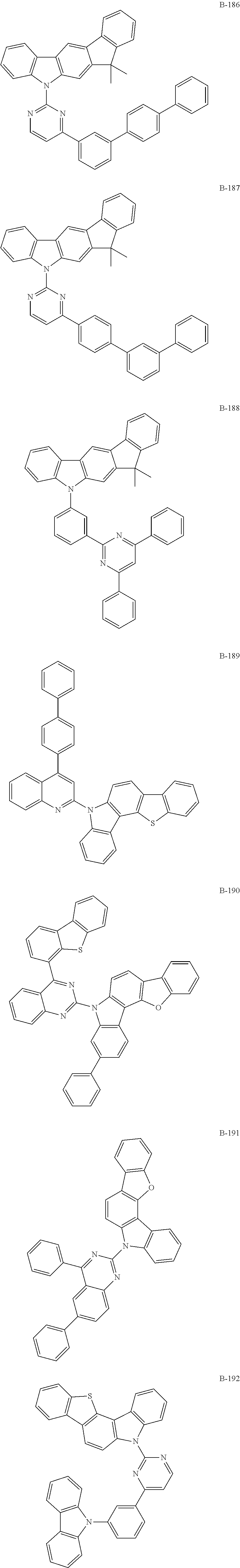

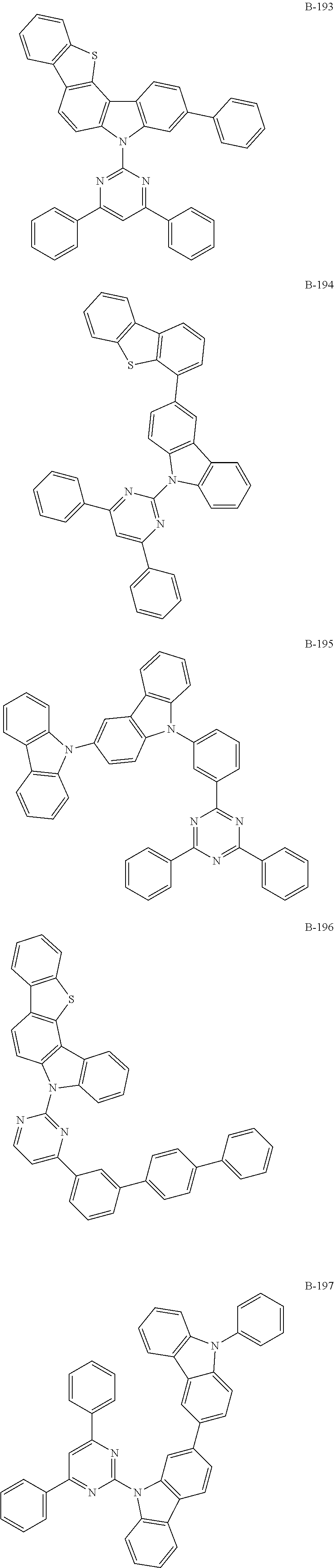

[wherein TPS represents a triphenylsilyl group]

The dopant used in the present invention is preferably at least one phosphorescent dopant. The dopant materials applied to the organic electroluminescent device according to the present invention are not limited, but may be preferably selected from metallated complex compounds of iridium, osmium, copper, and platinum, more preferably selected from ortho-metallated complex compounds of iridium, osmium, copper, and platinum, and even more preferably ortho-metallated iridium complex compounds.

The dopant comprised in the organic electroluminescent device of the present invention is preferably selected from the group consisting of the compounds of formulas 101 to 103 below.

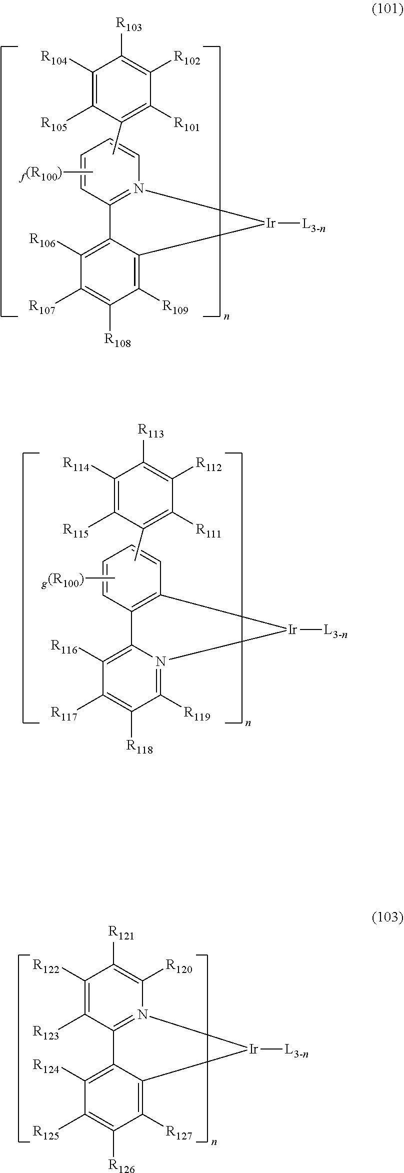

wherein L is selected from the following structures:

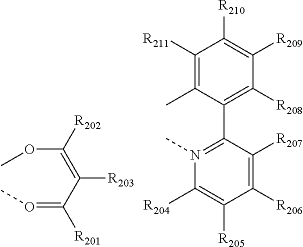

R₁₀₀ represents hydrogen, a substituted or unsubstituted (C1-C30)alkyl, or a substituted or unsubstituted (C3-C30)cycloalkyl;
R₁₀₁ to R₁₀₉ and R₁₁₁ to R₁₂₃ each independently represent hydrogen, deuterium, a halogen, a (C1-C30)alkyl unsubstituted or substituted with deuterium or a halogen(s), a cyano, a substituted or unsubstituted (C1-C30)alkoxy, a substituted or unsubstituted (C6-C30)aryl, or a substituted or unsubstituted (C3-C30)cycloalkyl; adjacent substituents of R₁₀₆ to R₁₀₉ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl; and adjacent substituents of R₁₂₀ to R₁₂₃ may be linked to each other to form a substituted or unsubstituted fused ring, e.g., quinoline unsubstituted or substituted with a halogen(s), alkyl, or aryl;
R₁₂₄ to R₁₂₇ each independently represent hydrogen, deuterium, a halogen, a substituted or unsubstituted (C1-C30)alkyl, or a substituted or unsubstituted (C6-C30)aryl; and adjacent substituents of R₁₂₄ to R₁₂₇ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;
R₂₀₁ to R₂₁₁ each independently represent hydrogen, deuterium, a halogen, a (C1-C30)alkyl unsubstituted or substituted with deuterium or a halogen(s), a substituted or unsubstituted (C3-C30)cycloalkyl, or a substituted or unsubstituted (C6-C30)aryl, and adjacent substituents of R₂₀₈ to R₂₁₁ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;
f and g each independently represent an integer of 1 to 3; where f or g is an integer of 2 or more, each of R₁₀₀ may be the same or different; and
n represents an integer of 1 to 3.

Specifically, the phosphorescent dopant compounds include the following:

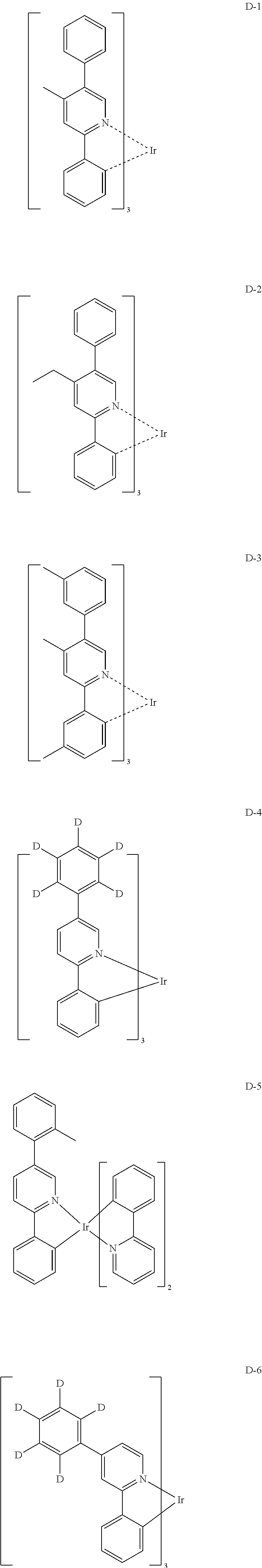

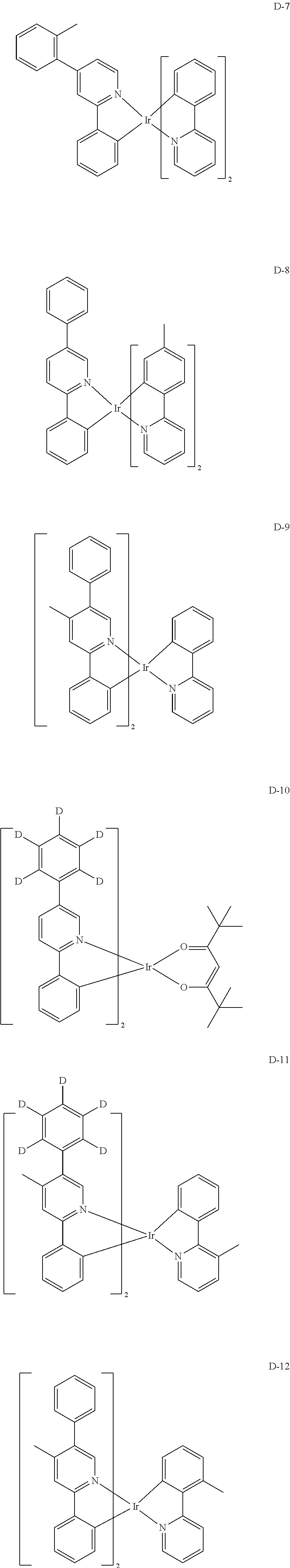

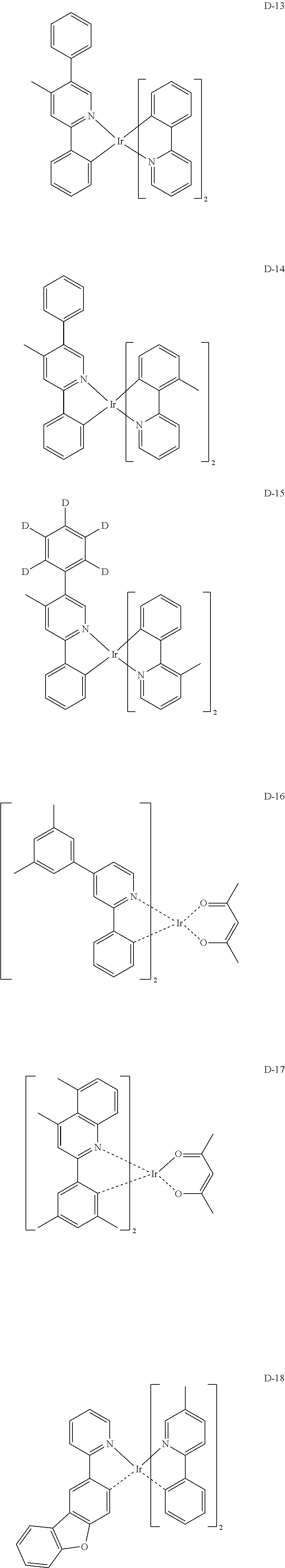

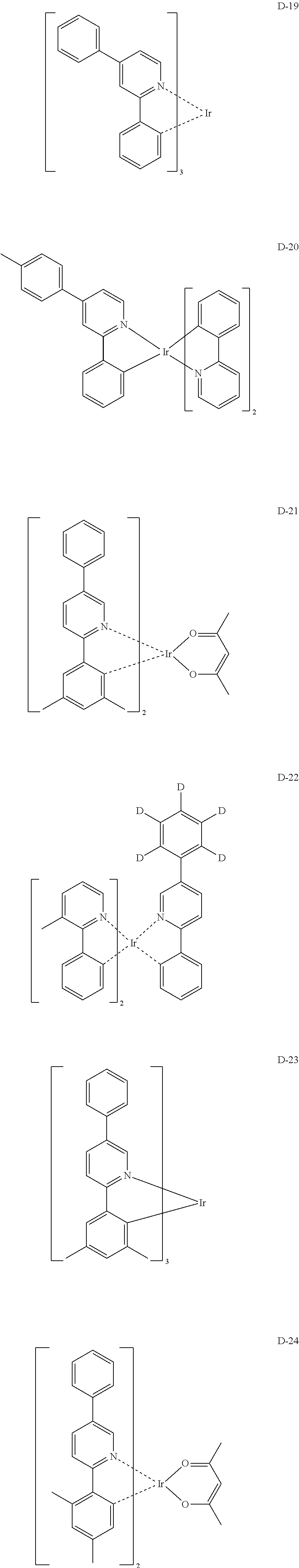

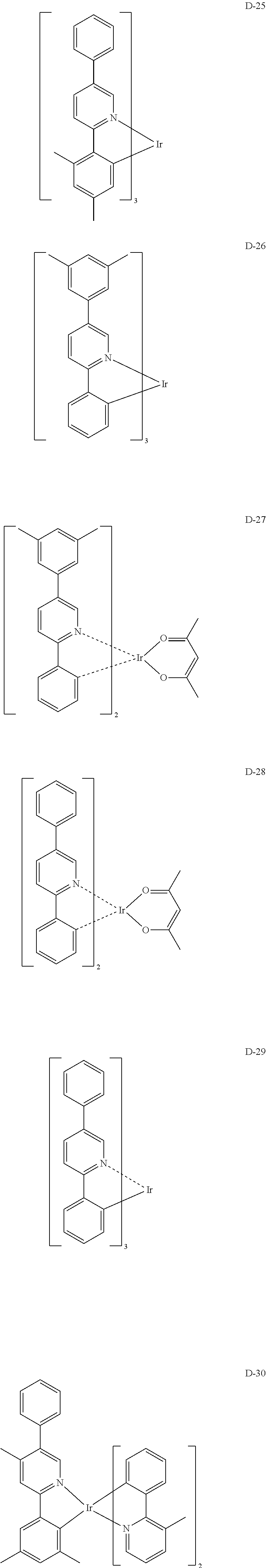

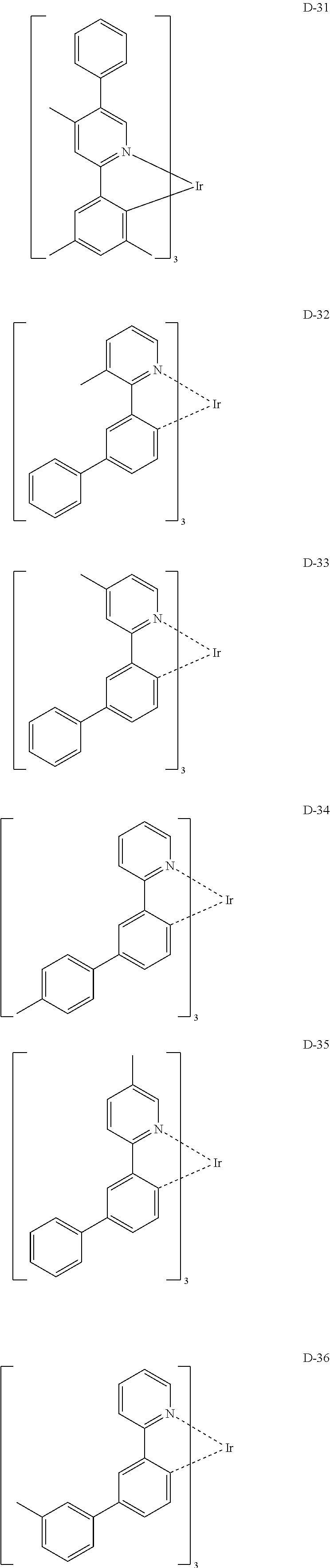

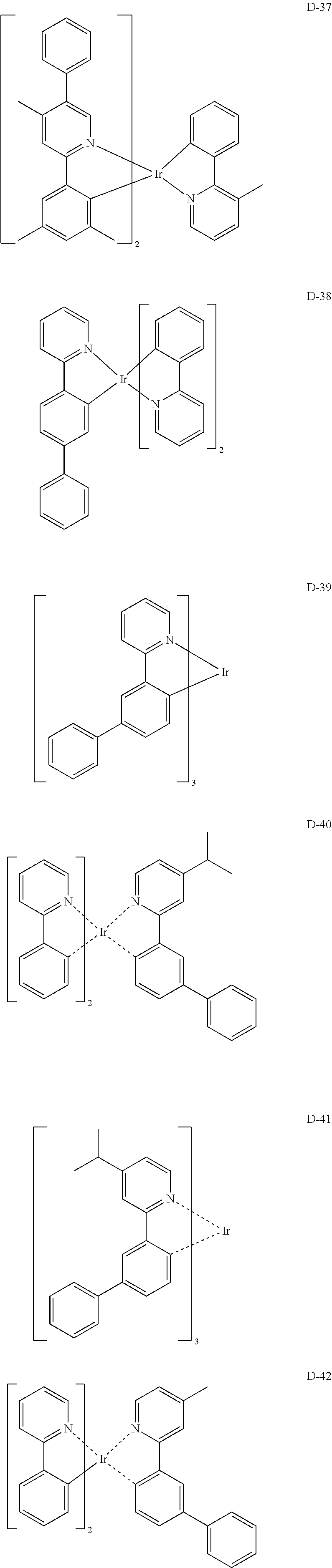

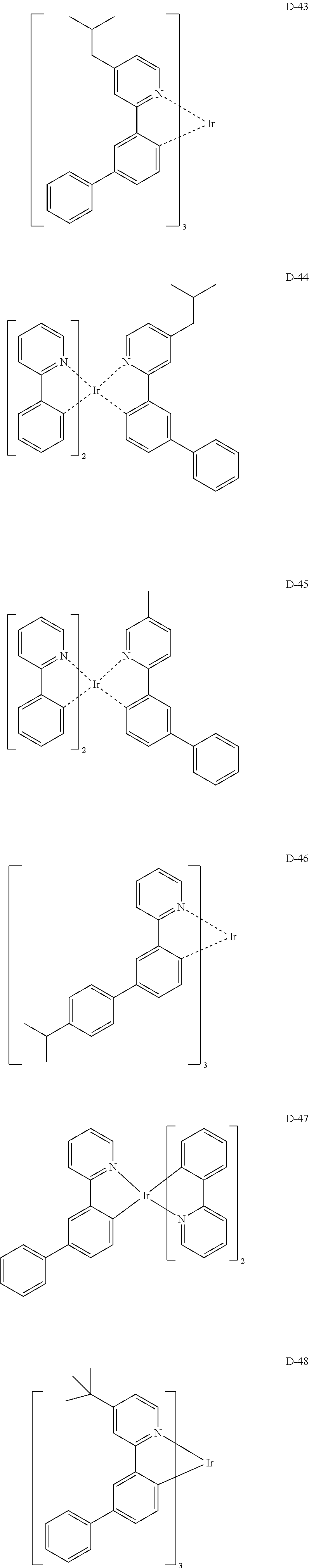

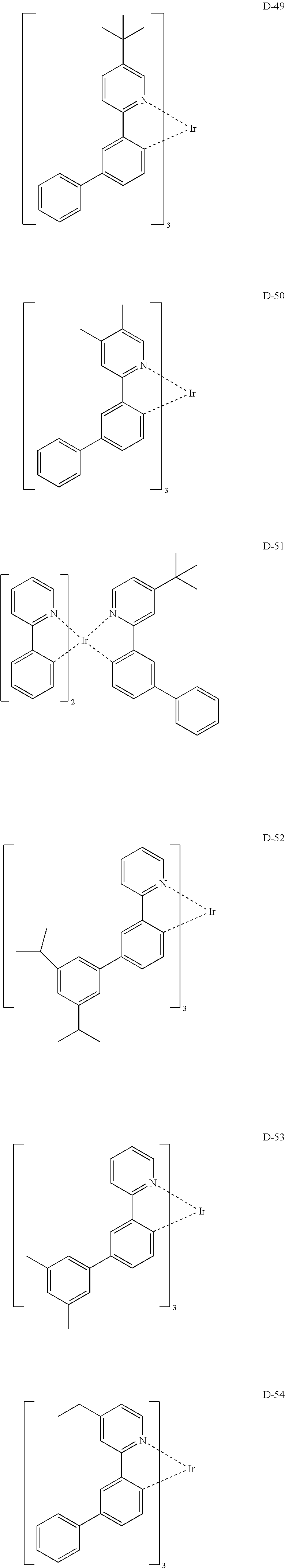

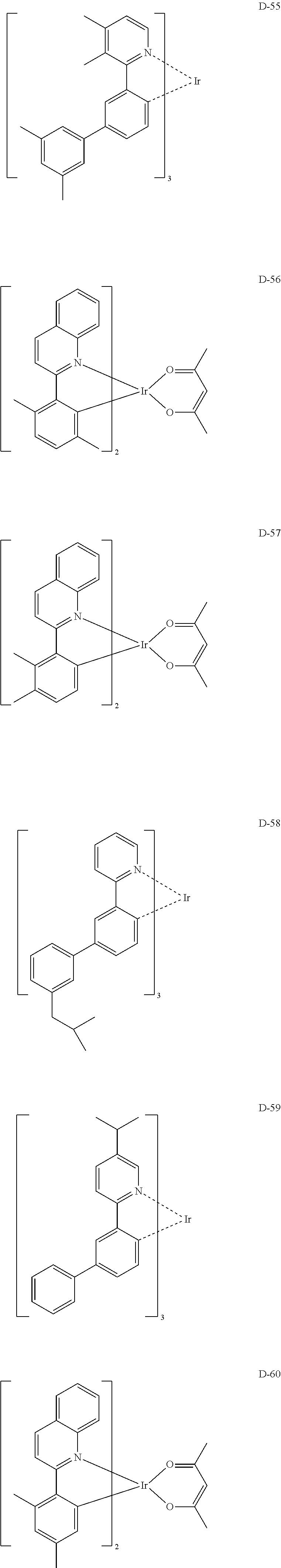

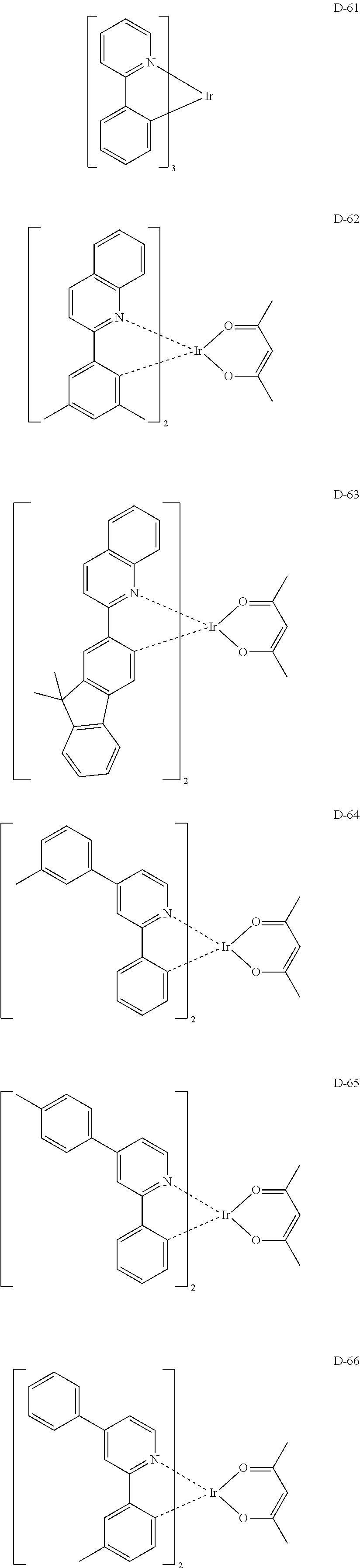

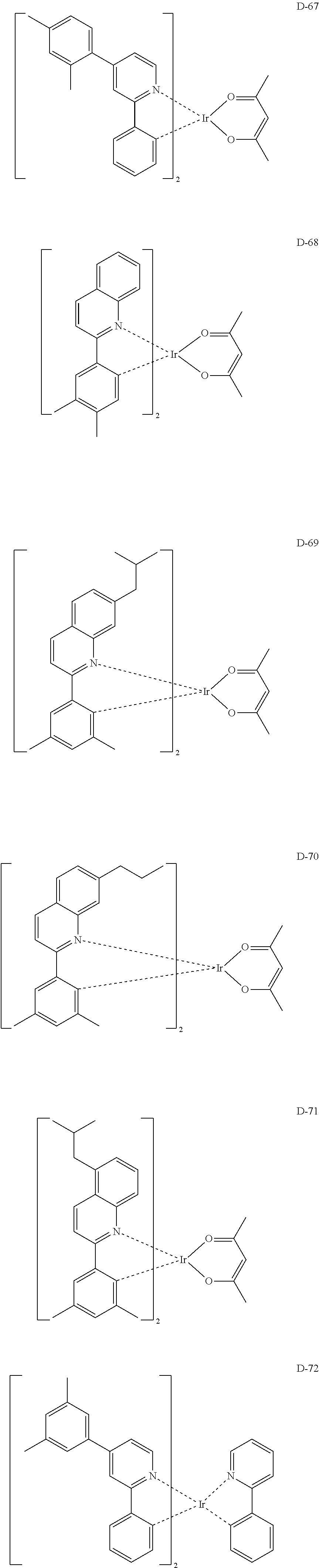

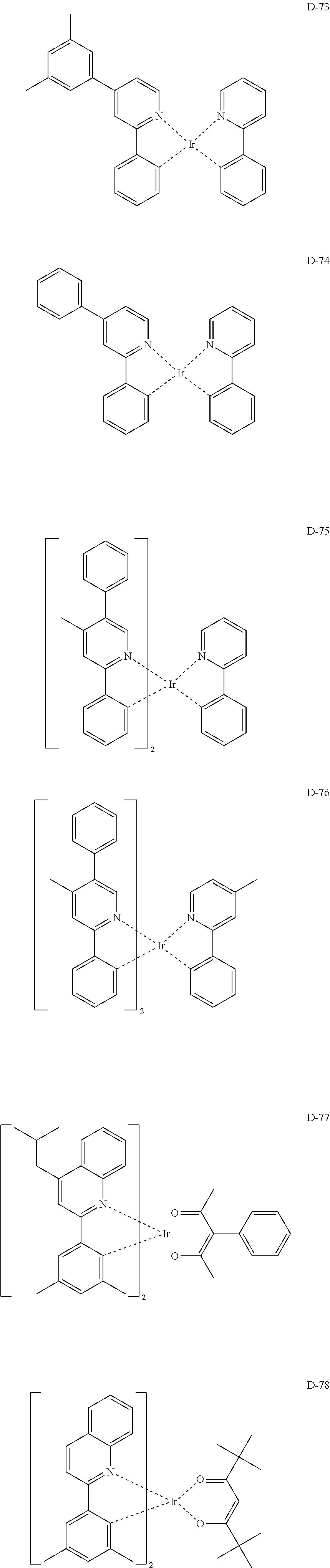

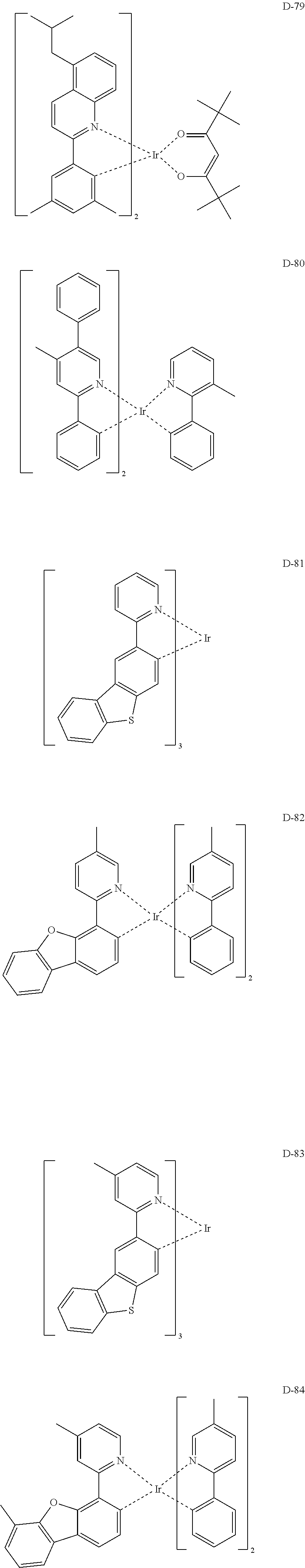

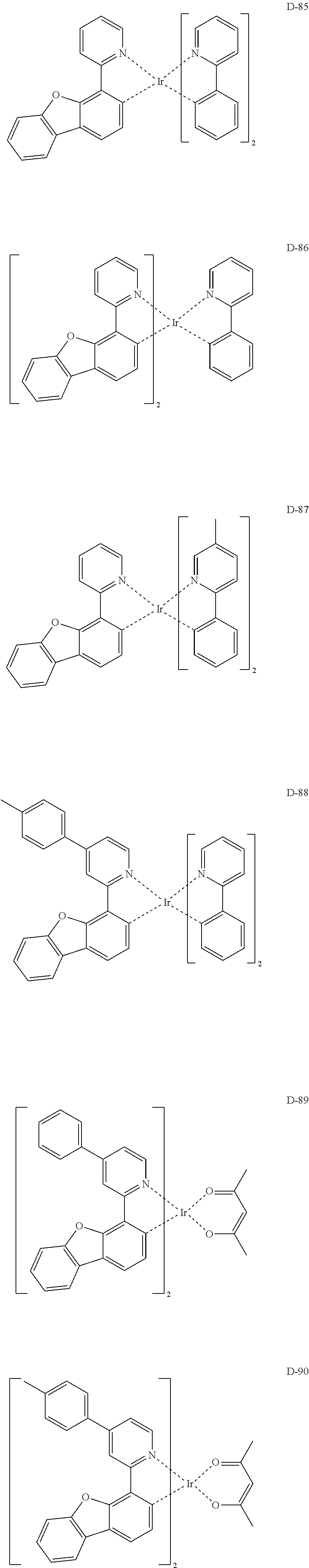

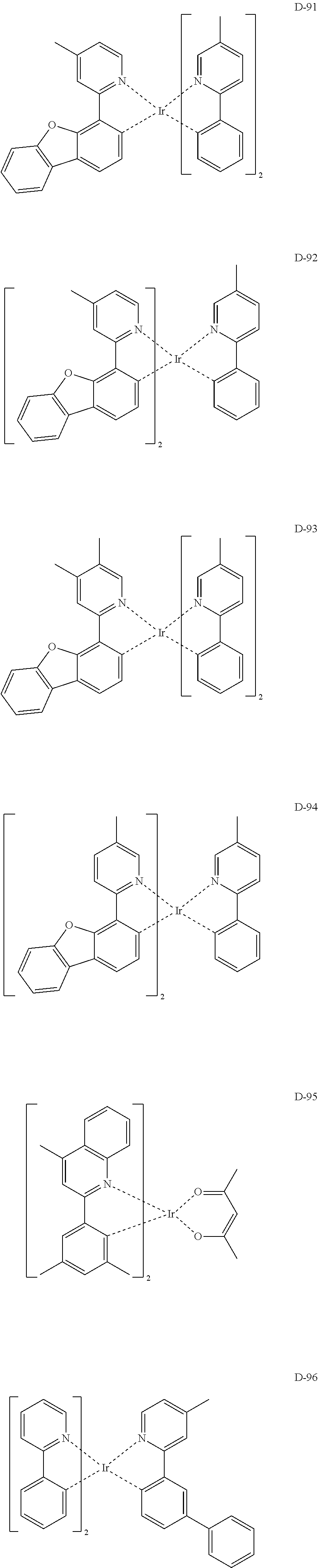

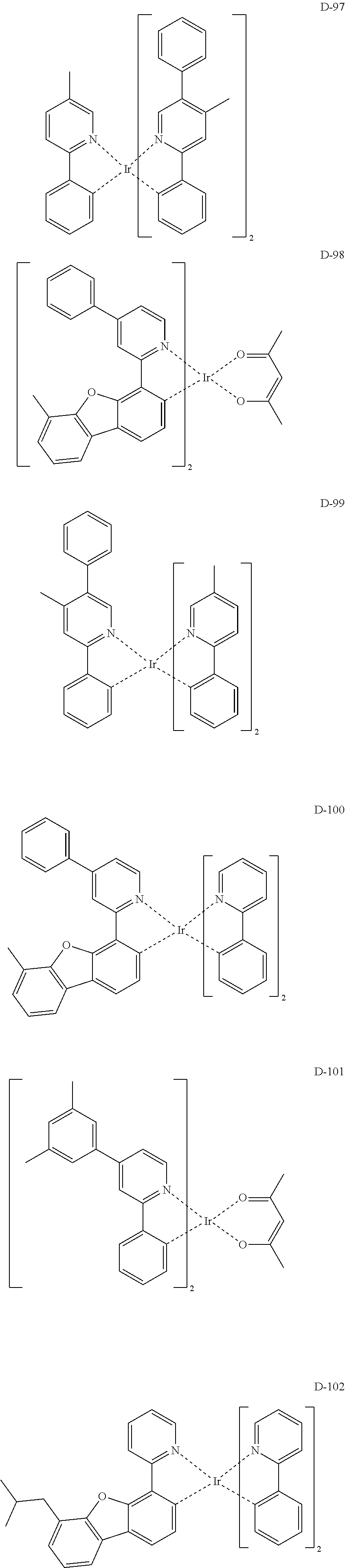

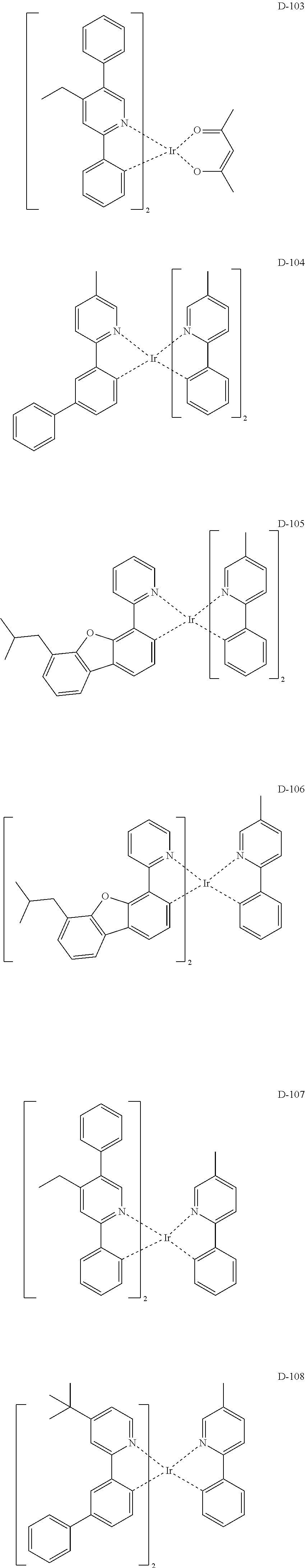

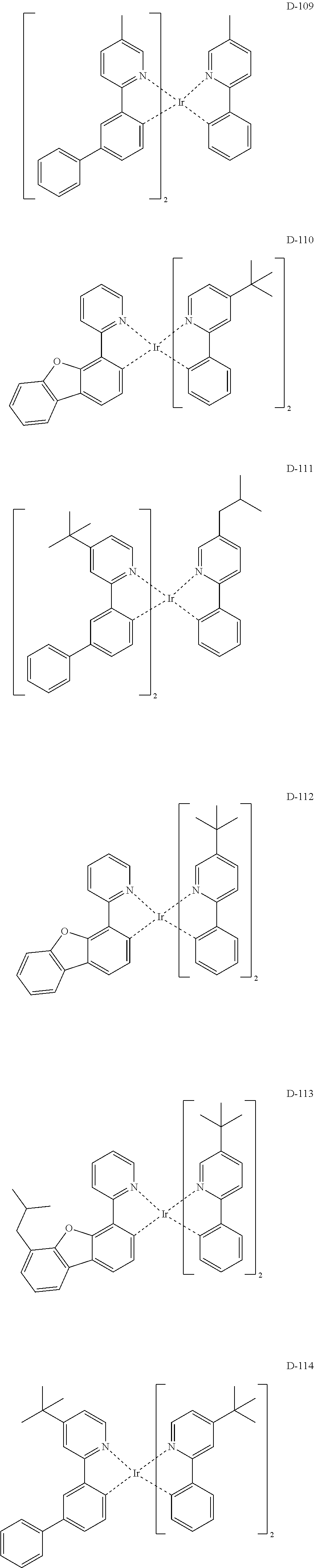

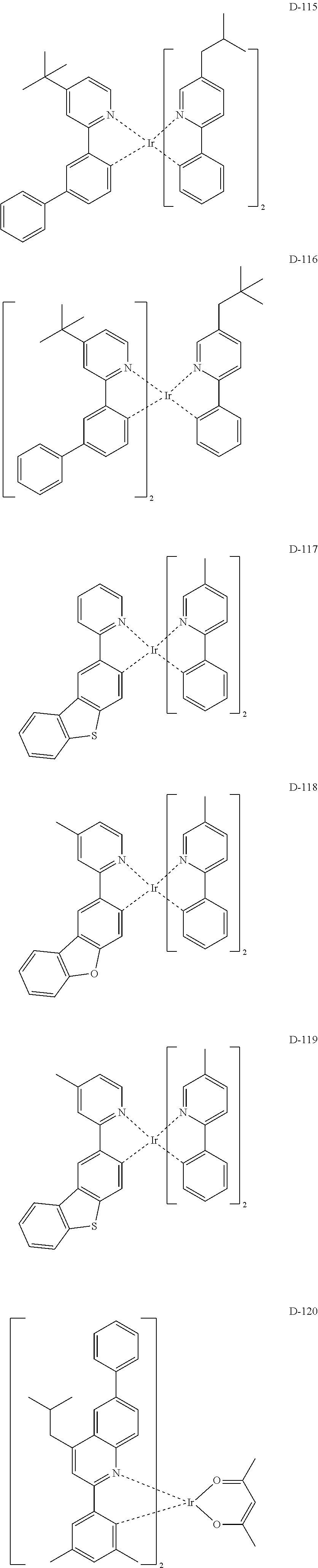

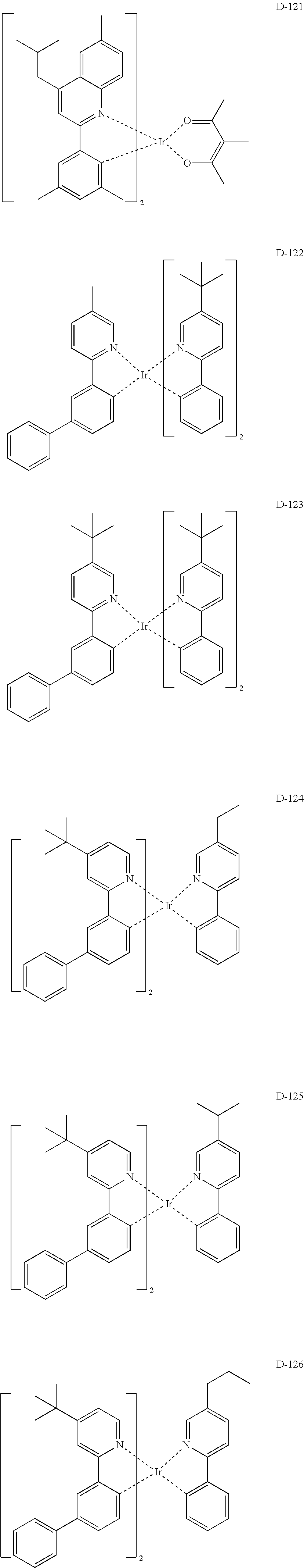

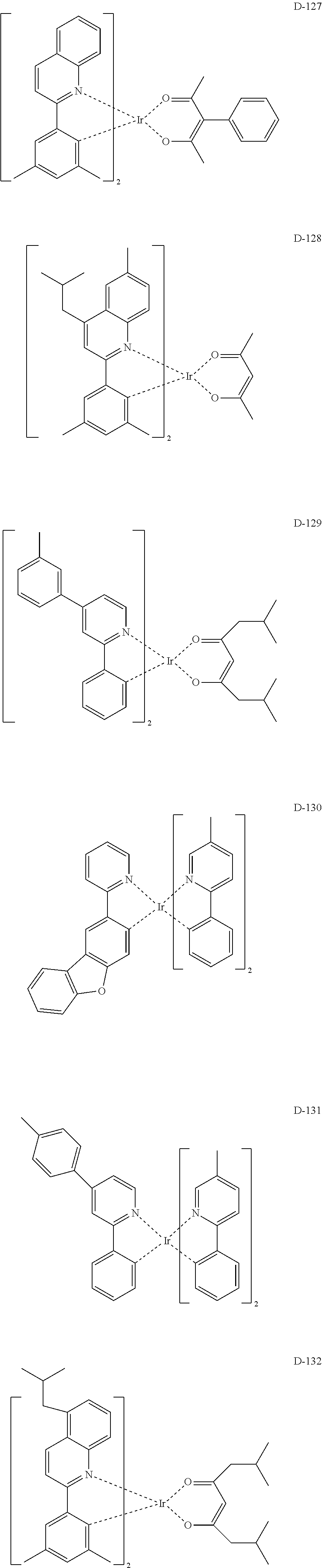

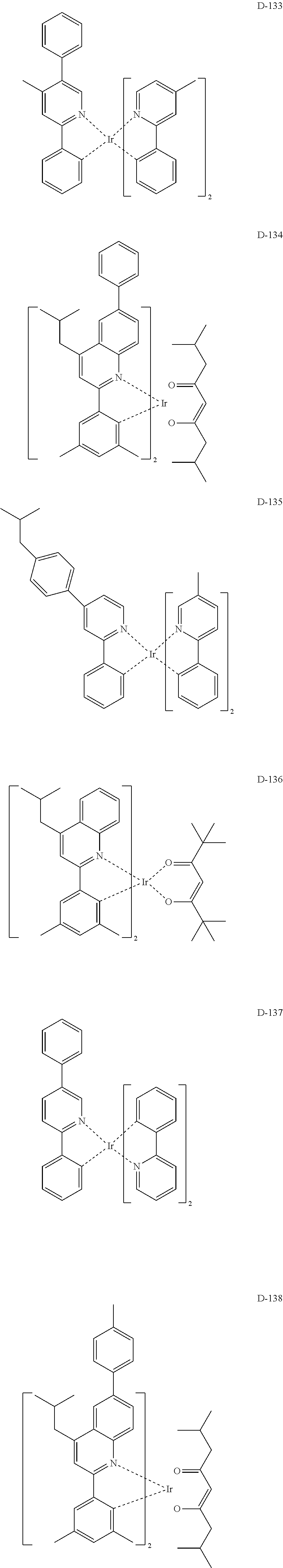

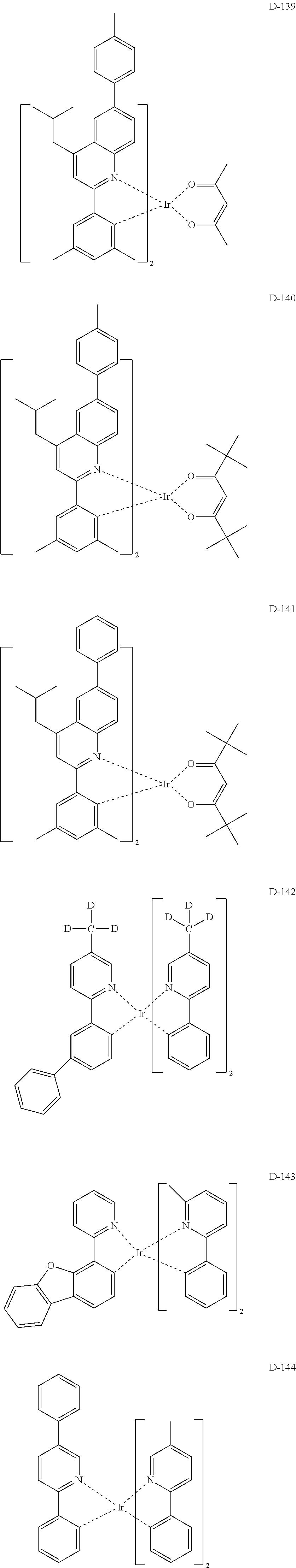

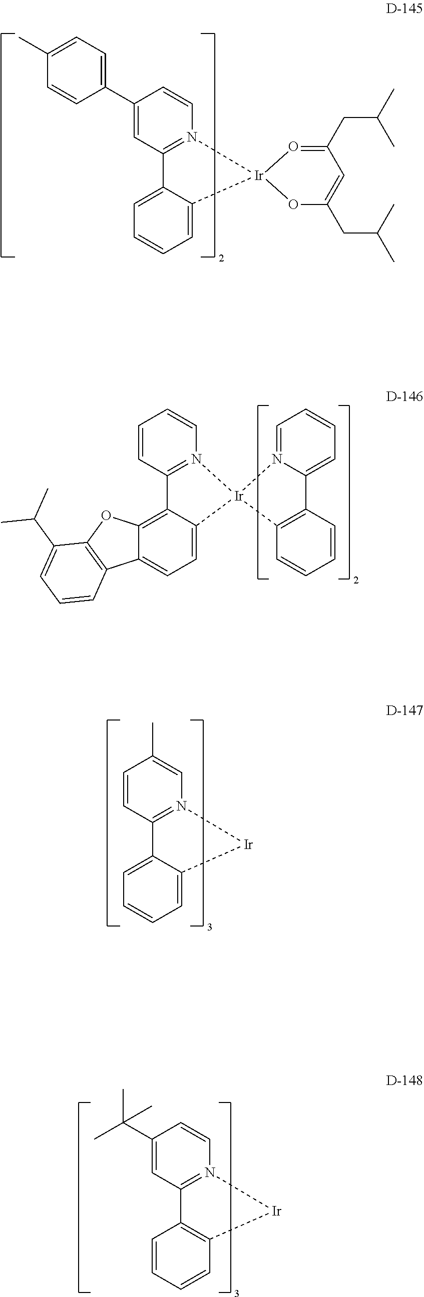

In another embodiment of the present invention, a plurality of host materials is provided. The plurality of host materials may comprise a compound represented by formula 1 and a compound represented by one of formulas 11 to 16.

In addition, the organic electroluminescent device according to the present invention comprises a first electrode; a second electrode; and at least one organic layer between the first and second electrodes. The organic layer may comprise the plurality of host materials.

In another embodiment of the present invention, a composition for preparing an organic electroluminescent device is provided. The composition comprises the phosphorous host material of the present invention.

In addition, the organic electroluminescent device according to the present invention comprises a first electrode; a second electrode; and at least one organic layer between the first and second electrodes. The organic layer comprises a light-emitting layer, and the light-emitting layer may comprise the composition for preparing the organic electroluminescent device according to the present invention.

The organic electroluminescent device according to the present invention may further comprise, in addition to the phosphorous host material of the present invention, at least one compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may further comprise a light-emitting layer and a charge generating layer.

In addition, the organic electroluminescent device according to the present invention may emit white light by further comprising at least one light-emitting layer which comprises a blue electroluminescent compound, a red electroluminescent compound or a green electroluminescent compound known in the field, besides the phosphorous host material according to the present invention. Also, if necessary, a yellow or orange light-emitting layer can be comprised in the device.

In the organic electroluminescent device according to the present invention, at least one layer (hereinafter, “a surface layer”) is preferably placed on an inner surface(s) of one or both electrode(s); selected from a chalcogenide layer, a metal halide layer, and a metal oxide layer. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiO_{X}(1≤X≤2), AlO_{X}(1≤X≤1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

In the organic electroluminescent device according to the present invention, a mixed region of an electron transport compound and reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

In order to form each layer of the organic electroluminescent device according to the present invention, dry film-forming methods such as vacuum evaporation, sputtering, plasma and ion plating methods, or wet film-forming methods such as spin coating, dip coating, and flow coating methods can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Hereinafter, the phosphorous host material and the luminescent properties of the device will be explained in detail with reference to the following examples.

### Example 1: Preparation of Compound C-1

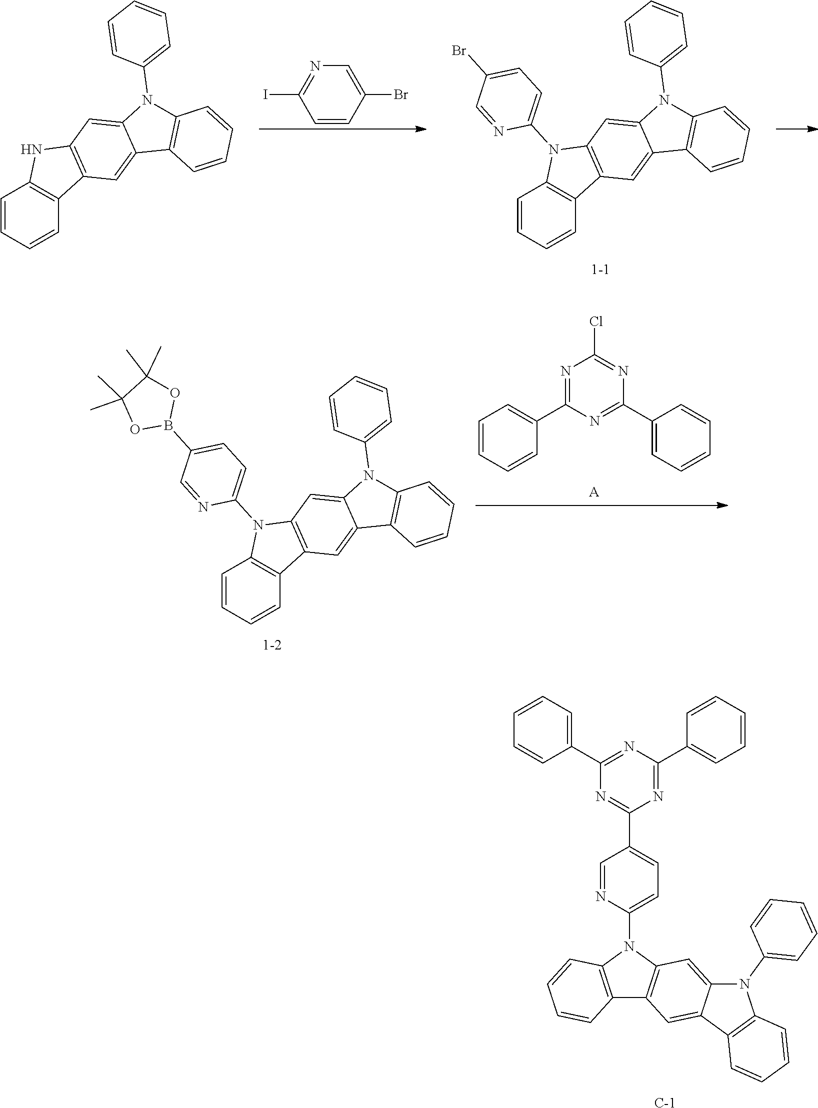

Preparation of Compound 1-1

After introducing 5-phenyl-5,7-dihydroindolo[2,3-b]carbazole (10 g, 29.72 mmol), 5-bromo-2-iodopyridine (12 g, 44.58 mmol), CuI (2.8 g, 14.86 mmol), K₃PO₄ (19 g, 89.46 mmol), ethylenediamine (EDA) (2 mL, 29.72 mmol), and toluene 150 mL into a flask, the mixture was stirred under reflux at 120° C. for 4 hours. After the reaction is completed, an organic layer was extracted with ethyl acetate, and remaining moisture was removed with magnesium sulfate and dried. The remaining product was then separated with column chromatography to obtain compound 1-1 (10.6 g, 75%).

Preparation of Compound 1-2

After introducing compound 1-1 (10.7 g, 21.91 mmol), bis(pinacolato)diborane (8.3 g, 32.86 mmol), PdCl₂(PPh₃)₂ (0.76 g, 1.096 mmol), KOAc (5.3 g, 54.77 mmol), and 1,4-dioxane 100 mL into a flask, the mixture was stirred under reflux at 120° C. for 2 hours. After the reaction is completed, an organic layer was extracted with ethyl acetate, and remaining moisture was removed with magnesium sulfate and dried. The remaining product was then separated with column chromatography to obtain compound 1-2 (7 g, 64%).

Preparation of Compound C-1

After introducing compound 1-2 (7 g, 19.30 mmol), compound A (4.2 g, 15.68 mmol), K₂CO₃ (5.4 g, 0.653 mmol), Pd(PPh₃)₄ (0.75 g, 0.65 mmol), purified water 35 mL, toluene 70 mL, and EtOH 35 mL into a flask, the mixture was stirred under reflux at 120° C. for 3 hours. After the reaction is completed, an organic layer was extracted with ethyl acetate, and remaining moisture was removed with magnesium sulfate and dried. The remaining product was then separated with column chromatography to obtain compound C-1 (5.2 g, 63%).

¹H NMR (600 MHz, CDCl₃, δ) 9.991 (s, 1H), 9.170-9.154 (d, J=9.6 Hz, 1H), 8.808-8.795 (m, 5H), 8.279-8.244 (m, 2H), 8.000 (d, J=12 Hz, 1H), 7.962 (s, 1H), 7.860 (d, J=6 Hz, 1H), 7.670-7.602 (m, 10H), 7.490-7.426 (m, 6H)

| | MW | UV | PL | M.P. |
|---|---|---|---|---|
| C-1 | 640.73 | 344 nm | 475 nm | 265.9° C. |

### Example 2: Preparation of Compound C-71

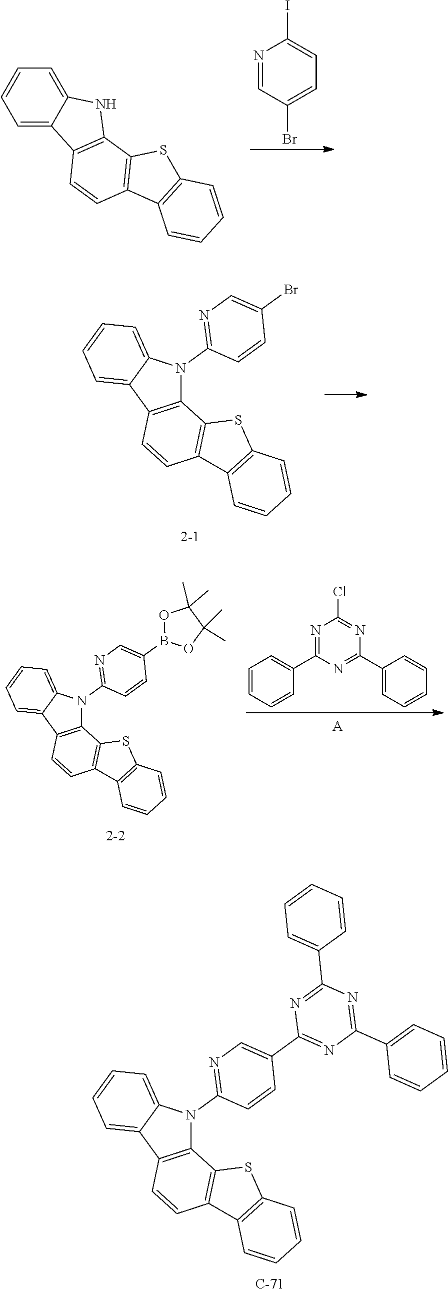

Preparation of Compound 2-1

After introducing 12H-benzo[4,5]thieno[2,3-a]carbazole (10 g, 36.58 mmol), 5-bromo-2-iodopyridine (20 g, 73.16 mmol), Cu (4.1 g, 65.84 mmol), Cs₂CO₃ (29 g, 91.45 mmol), and DCB 200 mL into a flask, the mixture was stirred under reflux at 200° C. for 4 hours. After the reaction is completed, an organic layer was extracted with ethyl acetate, and remaining moisture was removed with magnesium sulfate and dried. The remaining product was then separated with column chromatography to obtain compound 2-1 (12 g, 80%).

Preparation of Compound 2-2

After introducing compound 2-1 (12 g, 27.95 mmol), diborane (10 g, 41.93 mmol), PdCl₂(PPh₃)₂ (0.98 g, 1.398 mmol), KOAc (6.8 g, 69.87 mmol), and 1,4-dioxane 150 mL into a flask, the mixture was stirred under reflux at 120° C. for 2 hours. After the reaction is completed, an organic layer was extracted with ethyl acetate, and remaining moisture was removed with magnesium sulfate and dried. The remaining product was then separated with column chromatography to obtain compound 2-2 (6.5 g, 50%).

Preparation of Compound C-71

After introducing compound 2-2 (6.5 g, 13.64 mmol), compound A (6 g, 16.37 mmol), K₂CO₃ (5.6 g, 40.92 mmol), Pd(PPh₃)₄ (0.78 g, 0.682 mmol), 2 M K₂CO₃ 35 mL, toluene 70 mL, and EtOH 35 mL into a flask, the mixture was stirred under reflux at 120° C. for 3 hours. After the reaction is completed, an organic layer was extracted with ethyl acetate, and remaining moisture was removed with magnesium sulfate and dried. The remaining product was then separated with column chromatography to obtain compound C-71 (4.1 g, 52%).

¹H NMR (600 MHz, CDCl₃, δ) 10.21 (s, 1H), 9.358-9.345 (d, J=7.8 Hz, 1H), 8.869-8.856 (d, J=7.8 Hz, 4H), 8.286-8.219 (m, 4H), 7.839-7.807 (m, 3H), 7.699-7.673 (m, 6H), 7.523-7.486 (m, 2H), 7.455-7.439 (m, 2H)

| | MW | UV | PL | M.P. |
|---|---|---|---|---|
| C-71 | 581.68 | 332 nm | 469 nm | 326° C. |

### Example 3: Preparation of Compound C-84

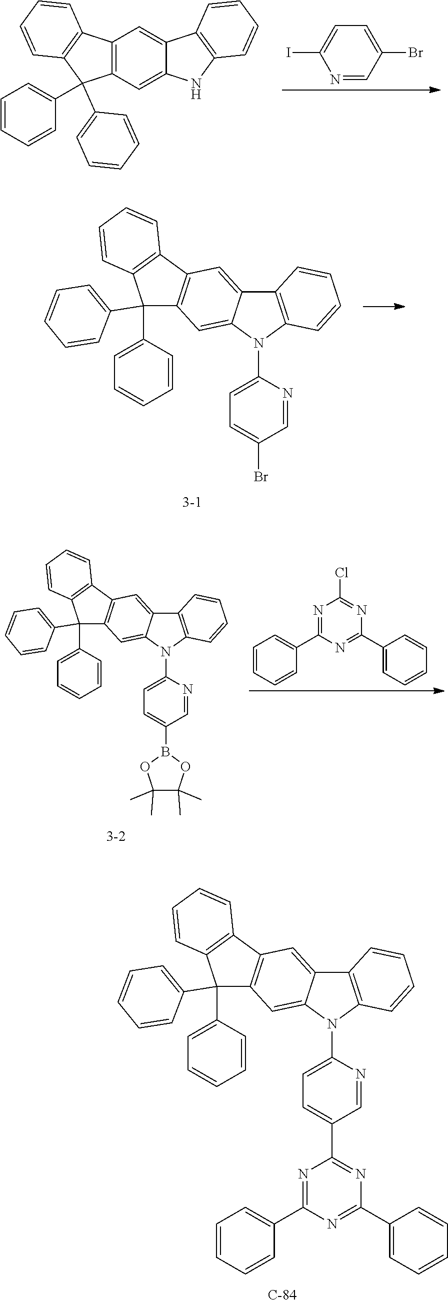

Preparation of Compound 3-1

After introducing 7,7-diphenyl-5,7-dihydroindeno[2,1-b]carbazole (10.0 g, 24.5 mmol), 5-bromo-2-iodopyridine (13.0 g, 45.9 mmol), CuI (3.4 g, 17.7 mmol), ethylenediamine (2.4 mL, 35.3 mmol), K₃PO₄ (22.5 g, 105.9 mmol), and toluene 180 mL into a flask, the mixture was stirred under reflux at 135° C. for 5 hours. After the reaction is completed, the mixture was extracted with methylene chloride (MC) and dried with MgSO₄. The remaining product was then separated with column chromatography, MeOH was added thereto, and the obtained solid was filtered under reduced pressure to obtain compound 3-1 (12.5 g, 90%).

Preparation of Compound 3-2

After introducing compound 3-1 (12 g, 21.3 mmol), dioxaborolane (10.4 g, 40.9 mmol), PdCl₂(PPh₃)₂ (1.9 g, 2.7 mmol), KOAc (5.4 g, 55 mmol), and 1,4-dioxane 140 mL into a flask, the mixture was stirred under reflux at 120° C. for 6 hours. After the reaction is completed, an organic layer was extracted with ethyl acetate, and remaining moisture was removed with magnesium sulfate and dried. The remaining product was then separated with column chromatography to obtain compound 3-2 (9.8 g, 75%).

Preparation of Compound C-84

After introducing compound 3-2 (9.3 g, 15.2 mmol), 2-chloro-4,6-diphenyl-1,3,5-triazine (6.1 g, 23.0 mmol), Pd(PPh₃)₄ (1.1 g, 0.96 mmol), K₂CO₃ (5.3 g, 38.3 mmol), toluene 80 mL, EtOH 20 mL, and H₂O 20 mL into a flask, the mixture was stirred under reflux at 120° C. for 5 hours. After the reaction is completed, an organic layer was extracted with ethyl acetate, and remaining moisture was removed with magnesium sulfate and dried. The remaining product was then separated with column chromatography to obtain compound C-84 (4.0 g, 37%).

¹H NMR (600 MHz, CDCl₃, δ) 9.96 (s, 1H), 9.107-9.089 (d, 1H), 8.800-8.788 (d, 4H), 8.460 (s, 1H), 8.190-8.177 (d, 1H), 8.039-8.025 (d, 1H), 8.004 (s, 1H), 7.913-7.899 (d, 1H), 7.699-7.685 (d, 1H), 7.653-7.587 (m, 6H), 7.477-7.452 (t, 1H), 7.411-7.356 (m, 4H), 7.301-7.191 (m, 10H)

| | MW | UV | PL | M.P. |
|---|---|---|---|---|
| C-84 | 715.86 | 360 nm | 521 nm | 327° C. |

### Example 4: Preparation of Compound C-46

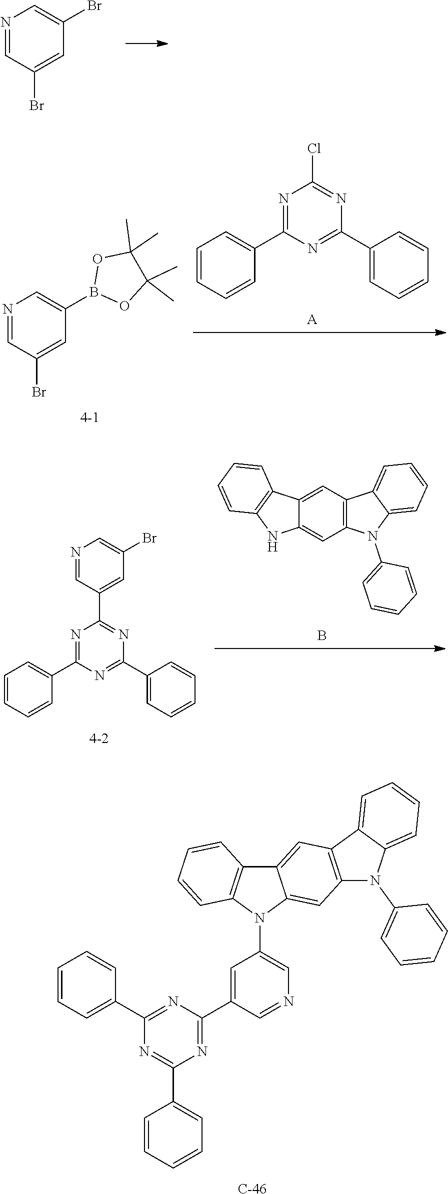

Preparation of Compound 4-1

After introducing 3,5-dibromopyridine (25 g, 105.5 mmol), diborane (28 g, 110.8 mmol), PdCl₂(PPh₃)₂ (3.7 g, 5.27 mmol), KOAc (20.7 g, 211 mmol), and 1,4-dioxane 527 mL into a flask, the mixture was stirred under reflux at 120° C. for 2 hours. After the reaction is completed, an organic layer was extracted with ethyl acetate, and remaining moisture was removed with magnesium sulfate and dried. The remaining product was then separated with column chromatography to obtain compound 4-1 (25 g, 86%).

Preparation of Compound 4-2

After introducing compound 4-1 (20 g, 70.4 mmol), compound A (12.5 g, 46.7 mmol), Cs₂CO₃ (30 g, 93.3 mmol), Pd(PPh₃)₄ (5 g, 4.67 mmol), toluene 150 mL, EtOH 50 mL, and H₂O 50 mL into a flask, the mixture was stirred under reflux at 120° C. for 3 hours. After the reaction is completed, an organic layer was extracted with ethyl acetate, and remaining moisture was removed with magnesium sulfate and dried. The remaining product was then separated with column chromatography to obtain compound 4-2 (7.5 g, 41%).

Preparation of Compound C-46

After dissolving compound 4-2 (7 g, 17.19 mmol), compound B (5 g, 15 mmol), Pd(OAc) (169 mg, 0.75 mmol), 2-dicyclohexylphosphino-2′,6′-dimethoxybiphenyl (S-phos) (617 mg, 1.5 mmol), and NaOtBu (3.6 g, 37.4 mmol) in xylene 150 mL, the mixture was stirred under reflux at 150° C. for 3 hours. After the reaction is completed, an organic layer was extracted with ethyl acetate, and remaining moisture was removed with magnesium sulfate and dried. The remaining product was then separated with column chromatography to obtain compound C-46 (4.8 g, 50%).

¹H NMR (600 MHz, CDCl₃, δ) 9.967 (s, 1H), 9.230-9.223 (t, J=1.92 Hz, 2.28 Hz, 1H), 9.092-9.088 (sd, J=2.46 Hz, 1H), 8.873 (s, 1H), 8.297-8.261 (dd, J=7.44 Hz, 7.38 Hz, 2H), 7.618-7.594 (m, 2H), 7.562-7.526 (m, 6H), 7.425-7.362 (m, 6H), 7.339-7.323 (m, 4H)

| | MW | UV | PL | M.P. |
|---|---|---|---|---|
| C-46 | 640.73 | 352 nm | 493 nm | 264° C. |

### Example 5: Preparation of Compound C-56

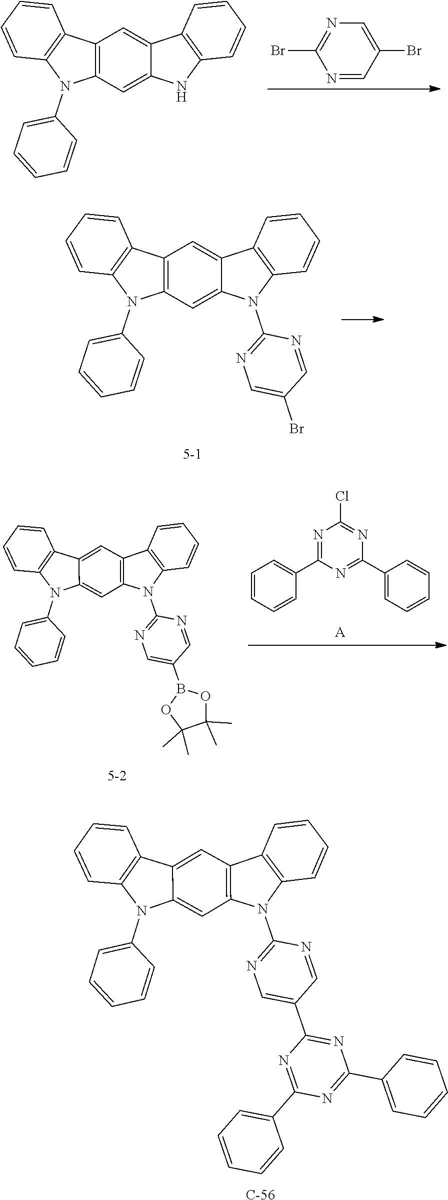

Preparation of Compound 5-1

After introducing 12H-benzo[4,5]thieno[2,3-a]carbazole (20 g, 60.2 mmol), 2,5-dibromopyrimidine (7.4 g, 50.2 mmol), dimethylaminopyridine (DMAP) (367 mg, 3.01 mmol), K₂CO₃ (25 g, 180.3 mmol), and dimethylformamide (DMF) 200 mL into a flask, the mixture was stirred at 50° C. for 8 hours. After the reaction is completed, an organic layer was extracted with ethyl acetate, and remaining moisture was removed with magnesium sulfate and dried. The remaining product was then separated with column chromatography to obtain compound 5-1 (23 g, 85.8%).

Preparation of Compound 5-2

After introducing compound 5-1 (18 g, 40.5 mmol), diborane (15.4 g, 60.71 mmol), PdCl₂(PPh₃)₂ (1.4 g, 2 mmol), KOAc (10 g, 101.3 mmol), and 1,4-dioxane 150 mL into a flask, the mixture was stirred under reflux at 120° C. for 12 hours. After the reaction is completed, an organic layer was extracted with ethyl acetate, and remaining moisture was removed with magnesium sulfate and dried. The remaining product was then separated with column chromatography to obtain compound 5-2 (6.3 g, 29%).

Preparation of Compound C-56

After introducing compound 5-2 (5.8 g, 10.8 mmol), compound A (3.5 g, 12.9 mmol), Cs₂CO₃ (8.8 g, 27 mmol), Pd(PPh₃)₄ (0.64 g, 0.54 mmol), H₂O 22 mL, toluene 44 mL, and EtOH 22 mL into a flask, the mixture was stirred under reflux at 120° C. for 2 hours. After the reaction is completed, an organic layer was extracted with ethyl acetate, and remaining moisture was removed with magnesium sulfate and dried. The remaining product was then separated with column chromatography to obtain compound C-56 (2 g, 28.8%).

¹H NMR (600 MHz, CDCl₃, δ) 9.98 (s, 2H), 9.148 (S, 1H) 8.7826 (S, 1H), 9.0922-9.07890 (d, J=7.98 Hz, 1H), 8.8088-8.79650 (d, J=7.38 Hz, 4H), 8.300-8.214 (dd, J=7.68 Hz, 2H), 7.7739-7.7483 (m, 4H), 7.734-7.349 (m, 12H)

| | MW | UV | PL | M.P. |
|---|---|---|---|---|
| C-56 | 641.73 | 302 nm | 500 nm | 347° C. |

### Example 6: Preparation of Compound C-115

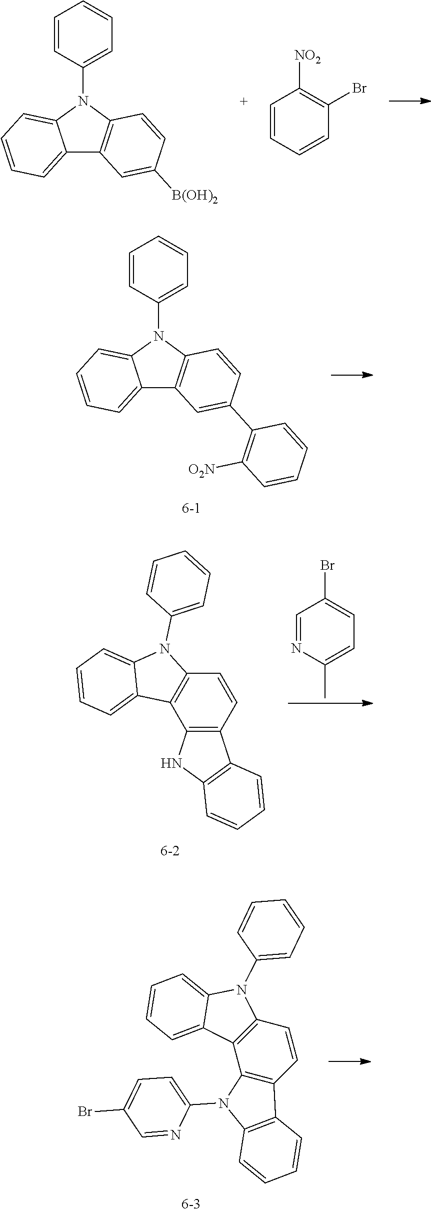

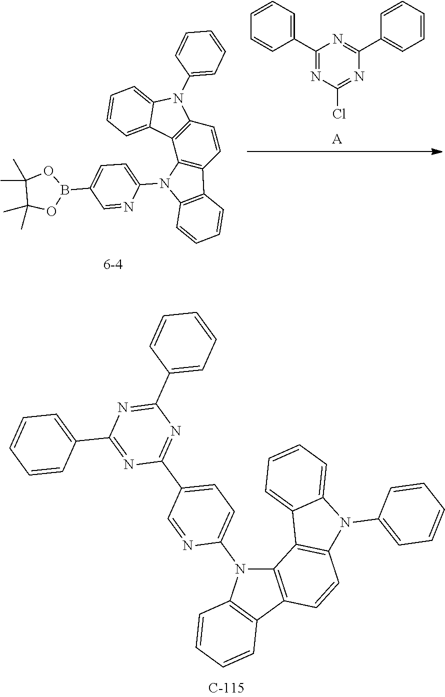

Preparation of Compound 6-1

After introducing (9-phenyl-9H-carbazol-3-yl)boronic acid (20 g, 69.65 mmol), 1-bromo-2-nitrobenzene (13 g, 63.32 mmol), Pd(PPh₃)₄ (4 g, 3.483 mmol), 2 M Na₂CO₃ 100 mL, toluene 310 mL, and EtOH 100 mL into a flask, the mixture was stirred under reflux at 120° C. for 3 hours. After the reaction is completed, an organic layer was extracted with ethyl acetate, and remaining moisture was removed with magnesium sulfate and dried. The remaining product was then separated with column chromatography to obtain compound 6-1 (23.7 g, 99%).

Preparation of Compound 6-2

After dissolving compound 6-1 (23.7 g, 65.03 mmol) and triphenylphosphine (43 g, 533 mmol) in dichlorobenzene 330 mL in a flask, the mixture was stirred under reflux at 150° C. for 6 hours. After the reaction is completed, the mixture was distilled and triturated with MeOH to obtain compound 6-2 (17 g, 80%).

Preparation of Compound 6-3

After introducing compound 6-2 (17 g, 51.14 mmol), 5-bromo-2-iodopyridine (28 g, 102.28 mmol), Cu (6 g, 92.05 mmol), Cs₂CO₃ (41 g, 127.85 mmol), and DCB 260 mL into a flask, the mixture was stirred under reflux at 200° C. for 4 hours. After the reaction is completed, an organic layer was extracted with ethyl acetate, and remaining moisture was removed with magnesium sulfate and dried. The remaining product was then separated with column chromatography to obtain compound 6-3 (17 g, 71%).

Preparation of Compound 6-4

After introducing compound 6-3 (17 g, 34.81 mmol), diborane (13 g, 52.21 mmol), PdCl₂(PPh₃)₂ (1.2 g, 1.740 mmol), KOAc (8.5 g, 87.02 mmol), and 1,4-dioxane 180 mL into a flask, the mixture was stirred under reflux at 120° C. for 2 hours. After the reaction is completed, an organic layer was extracted with ethyl acetate, and remaining moisture was removed with magnesium sulfate and dried. The remaining product was then separated with column chromatography to obtain compound 6-4 (18 g, 99%).

Preparation of Compound C-115

After introducing compound 6-4 (10 g, 18.67 mmol), compound A (8.2 g, 22.41 mmol), Pd(PPh₃)₄ (1.0 g, 0.933 mmol), 2 M Na₂CO₃ 50 mL, toluene 100 mL, and EtOH 50 mL into a flask, the mixture was stirred under reflux at 120° C. for 3 hours. After the reaction is completed, an organic layer was extracted with ethyl acetate, and remaining moisture was removed with magnesium sulfate and dried. The remaining product was then separated with column chromatography to obtain compound C-115 (1.3 g, 11%).

¹H NMR (600 MHz, CDCl₃, δ) 10.190 (s, 1H), 9.163-9.150 (d, J=8.3 Hz, 1H), 8.134-8.800 (m, 3H), 8.183-8.082 (m, 2H), 7.683-7.538 (m, 14H), 7.453-7.272 (m, 5H), 6.901-6.877 (m, 1H), 6.431-6.417 (d, J=8.4 Hz, 1H)

| | MW | UV | PL | M.P. |
|---|---|---|---|---|
| C-115 | 640.73 | 344 nm | 657 nm | 312° C. |

### Example 7: Preparation of Compound C-118

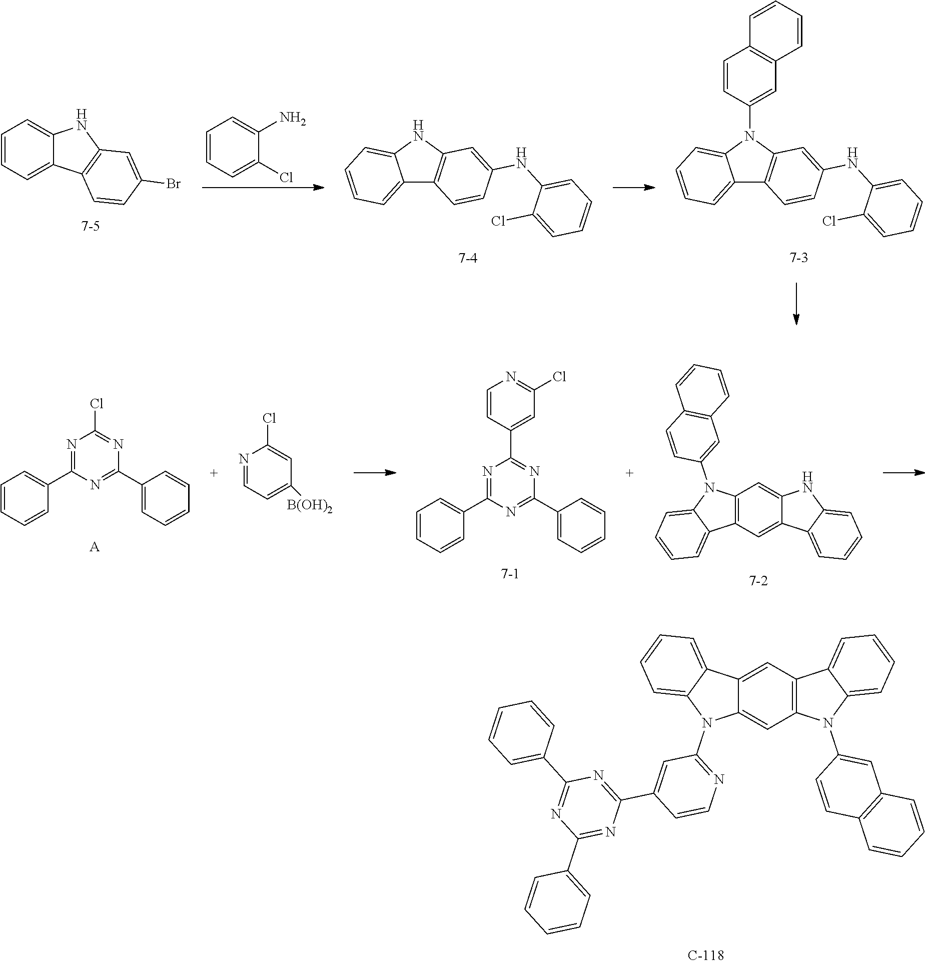

Preparation of Compound 7-4

After introducing compound 7-5 (2-bromocarbazole) (100 g, 406 mmol), 2-chloroaniline (85.47 mL, 813 mmol), Pd₂(dba)₃ (11.16 g, 12 mmol), P(t-Bu)₃ (4.9 g, 24 mmol), and NaOtBu (117.16 g, 1219 mmol) into a flask, toluene 2 L was added thereto to dissolve the mixture, and the mixture was stirred under reflux for 48 hours. After the reaction is completed, the reactant was extracted with ethyl acetate, and remaining moisture was removed with magnesium sulfate and dried. The remaining product was then purified with column chromatography to obtain compound 7-4 (89 g, yield: 74%).

Preparation of Compound 7-3

After introducing compound 7-4 (89 g, 304 mmol), 2-bromonaphthalene (75 g, 365 mmol), CuI (29 g, 152 mmol), 1,2-cyclohexanediamine (34.7 g, 304 mmol), and Cs₂CO₃ (198 g, 608 mmol) into a flask, xylene was added thereto to dissolve the mixture, and the mixture was stirred under reflux at 150° C. for 6 hours. After the reaction is completed, the reactant was filtered through celite, and the filtrate was then purified with column chromatography to obtain compound 7-3 (71 g, yield: 55%).

Preparation of Compound 7-2

After introducing compound 7-3 (71 g, 169 mmol), Pd(OAc)₂ (3.8 g, 17 mmol), PCy₃HBF₄ (18.72 g, 51 mmol), and Cs₂CO₃ (165 g, 508 mmol) into a flask, dimethylamide (DMA) was added thereto to dissolve the mixture, and the mixture was stirred under reflux at 200° C. for 2 hours. After the reaction is completed, the reactant was extracted with ethyl acetate, and remaining moisture was removed with magnesium sulfate and dried. The remaining product was then purified with column chromatography to obtain compound 7-2 (30 g, yield: 46%).

Preparation of Compound 7-1

After dissolving compound A (12.76 g, 48 mmol), 2-chloropyridine-4-boronic acid (5 g, 32 mmol), Pd(PPhs)₄ (1.8 g, 2 mmol), and K₂CO₃ (8.7 g, 64 mmol) in a mixture solvent of ethanol 31 mL, water 31 mL, and toluene 100 mL in a flask, the mixture was stirred under reflux at 120° C. for 4 hours. After the reaction is completed, the reactant was extracted with ethyl acetate, and remaining moisture was removed with magnesium sulfate and dried. The remaining product was then purified with column chromatography to obtain compound 7-1 (4.6 g, yield: 41%).

Preparation of Compound C-118

After dissolving compound 7-2 (4.2 g, 11 mmol), compound 7-1 (4.6 g, 13 mmol), Pd(OAc)₂ (123 mg, 0.54 mmol), S-Phos (451 mg, 1 mmol), and NaOtBu (2.63 g, 27 mmol) in xylene 110 mL in a flask, the mixture was stirred under reflux at 150° C. for 3 hours. After the reaction is completed, the produced solid was filtered and dried. The dried solid was dissolved in chloroform, purified with a silica gel filter, and recrystallized with methanol to obtain compound C-118 (2.4 g, yield: 31%).

¹H NMR (600 MHz, CDCl₃, δ) 8.974 (s, 1H), 8.868 (s, 1H), 8.851-8.842 (d, 1H, J=5.2 HZ), 8.748-8.735 (sd, 4H, J=7.44 Hz), 8.467-8.458 (sd, 1H, J=5.1 Hz), 8.295-8.270 (m, 2H), 8.030 (s, 1H), 7.947-7.933 (d, 1H, J=8.16 Hz), 7.899 (s, 1H), 7.753-7.715 (m, 2H), 7.688-7.607 (m, 4H), 7.578-7.553 (m, 4H), 7.461-7.324 (m, 7H)

| | MW | UV | PL | M.P. |
|---|---|---|---|---|
| C-118 | 690.79 | 356 nm | 532 nm | 316° C. |

### Example 8: Preparation of Compound C-123

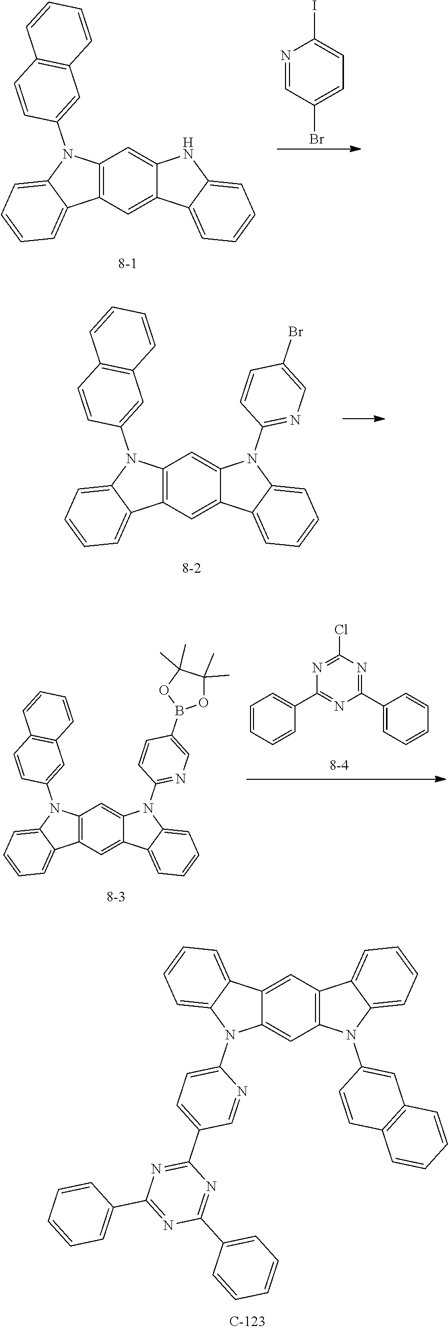

Preparation of Compound 8-2

After introducing compound 8-1 (10 g, 26 mmol), 1-iodo-4-bromopyridine (14.85 g, 52 mmol), CuI (2.5 g, 13 mmol), EDA (1.5 g, 26 mmol), and K₃PO₄ (16.7 g, 78 mmol) into a flask, toluene 130 mL was added thereto to dissolve the mixture, and the mixture was stirred under reflux for 3 hours. After the reaction is completed, the reactant was filtered through celite and extracted with dichloromethane. The remaining moisture was removed with magnesium sulfate and dried. The remaining product was then purified with column chromatography to obtain compound 8-2 (7.7 g, yield: 55%).

Preparation of Compound 8-3

After introducing compound 8-2 (8.6 g, 16 mmol), 4,4,4′,4′,5,5,5′,5′-oxamethyl-2,2′-bi(1,3,2-dioxaborolane) (4.4 g, 18 mmol), PdCl₂(PPh₃)₂ (448 mg, 0.639 mmol), KOAc (6.9 g, 70 mmol) into a flask, 1,4-dioxane was added thereto to dissolve the mixture, and the mixture was stirred under reflux for 3 hours. After the reaction is completed, the reactant was filtered through celite and extracted with dichloromethane. The remaining moisture was removed with magnesium sulfate and dried. The remaining product was then purified with column chromatography to obtain compound 8-3 (6.7 g, yield: 71%).

Preparation of Compound C-123

After dissolving compound 8-3 (5.3 g, 9 mmol), compound 8-4 (2.2 g, 8 mmol), Pd(PPh₃)₄ (950 mg, 0.822 mmol), and K₂CO₃ (3.4 g, 25 mmol) in a mixture solvent of ethanol 13 mL, water 13 mL, and toluene 100 mL in a flask, the mixture was stirred under reflux at 120° C. for 3 hours. After the reaction is completed, the produced solid was filtered and dried. The dried solid was dissolved in chloroform, purified with a silica gel filter, and recrystallized with methanol to obtain compound C-123 (2.0 g, yield: 35%).

¹H NMR (600 MHz, CDCl₃, δ) 9.920-9.916 (sd, 1H, J=2.28 Hz), 9.102-9.085 (dd, 1H, J=8.37 Hz), 8.834 (s, 1H), 8.751-8.739 (sd, 4H, J=7.44 Hz), 8.285-8.237 (dd, 2H, J=21.36 Hz), 8.120 (s, 1H), 8.090-8.076 (d, 1H, J=8.52 Hz), 7.986 (s, 1H), 7.972-7.925 (m, 3H), 7.834-7.821 (d, 1H, J=8.52 Hz), 7.739-7.721 (dd, 1H, J=8.52 Hz), 7.635-7.555 (m, 8H), 7.450-7.325 (m, 5H)

| | MW | UV | PL | M.P. |
|---|---|---|---|---|
| C-123 | 690.79 | 304 nm | 482 nm | 336° C. |

### Example 9: Preparation of Compound C-128

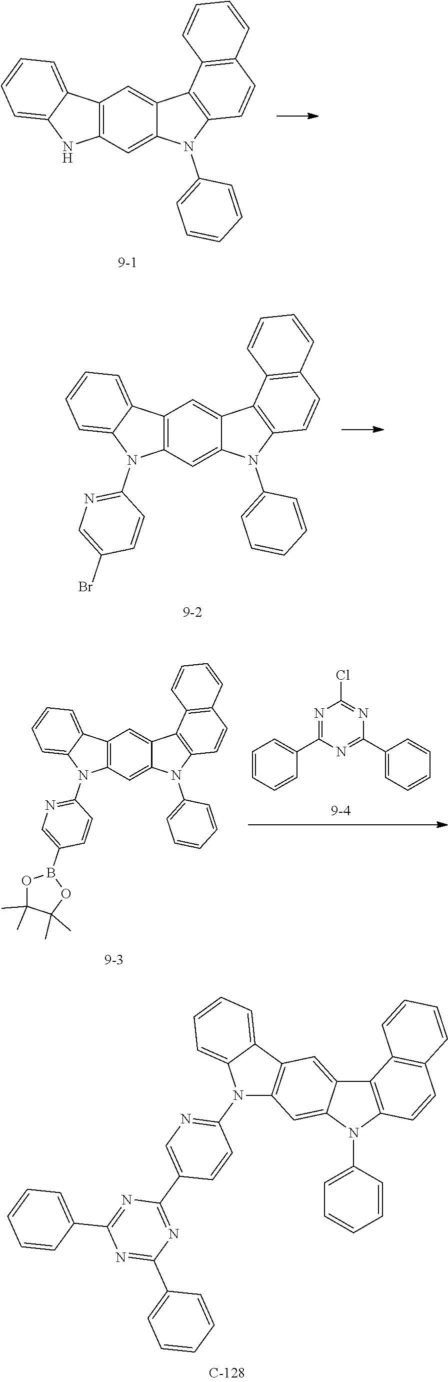

Preparation of Compound 9-2

After introducing compound 9-1 (7 g, 18 mmol), 2-iodo-5-bromopyridine (10.4 g, 37 mmol), CuI (1.74 g, 9 mmol), EDA (1.1 g, 18 mmol), and K₃PO₄ (11.7 g, 55 mmol) into a flask, toluene 100 mL was added thereto to dissolve the mixture, and the mixture was stirred under reflux at 120° C. After the reaction is completed, the reactant was cooled at room temperature, filtered through celite, and an organic layer was extracted with ethyl acetate. The remaining moisture was removed with magnesium sulfate and dried. The remaining product was then purified with column chromatography to obtain compound 9-2 (8.6 g, yield: 87%).

Preparation of Compound 9-3

After introducing compound 9-2 (8.6 g, 16 mmol), 4,4,4′,4′,5,5,5′,5′-oxamethyl-2,2′-bi(1,3,2-dioxaborolane) (4.4 g, 18 mmol), PdCl₂(PPh₃)₂ (0.448 g, 0.638 mmol), and KOAc (6.9 g, 70 mmol) into a flask, 1,4-dioxane 110 mL was added thereto to dissolve the mixture, and the mixture was stirred under reflux at 110° C. After the reaction is completed, the reactant was cooled at room temperature, filtered through celite, and an organic layer was extracted with ethyl acetate. The remaining moisture was removed with magnesium sulfate and dried. The remaining product was then purified with column chromatography to obtain compound 9-3 (6.7 g, yield: 71%).

Preparation of Compound C-128

After dissolving compound 9-3 (6.7 g, 11 mmol), compound 9-4 (3.6 g, 14 mmol), Pd(PPh₃)₄ (661 mg, 0.572 mmol), and K₂CO₃ (4.7 g, 34 mmol) in a mixture solvent of toluene 50 mL, EtOH 17 mL, and H₂O 17 mL in a flask, the mixture was stirred under reflux at 120° C. After the reaction is completed, the produced solid was cooled at room temperature, filtered, and dried. The solid was dissolved in chloroform, purified with a silica gel filter, and recrystallized with methanol to obtain compound C-128 (4.5 g, yield: 57%).

¹H NMR (600 MHz, CDCl₃, δ) 9.977-9.973 (sd, 1H, J=2.22 Hz), 9.294 (s, 1H), 9.155-9.138 (d, 1H, J=8.4 Hz), 9.053-9.039 (d, 1H, J=8.4 Hz), 8.792-8.778 (sd, 4H, J=7.89 Hz), 8.378-8.366 (d, 1H, J=7.44 Hz), 8.049-8.023 (m, 3H), 7.880-7.867 (d, 1H, J=8.34 Hz), 7.837-7.813 (m, 2H), 7.667-7.587 (m, 10H), 7.562-7.511 (m, 3H), 7.483-7.407 (m, 2H)

| | MW | UV | PL | M.P. |
|---|---|---|---|---|
| C-128 | 690.79 | 344 nm | 499 nm | 277° C. |

### Example 10: Preparation of Compound C-8

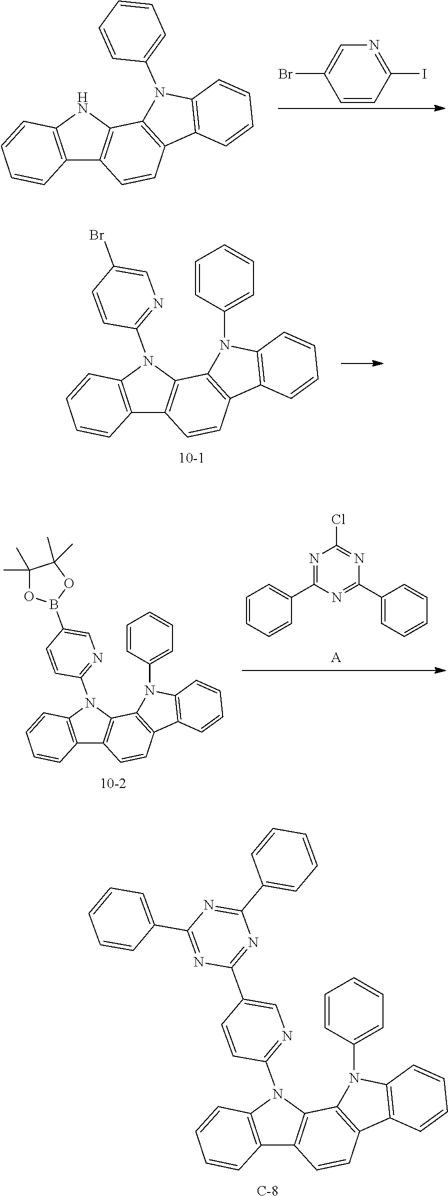

Preparation of Compound 10-1

After dissolving 11-phenyl-11,12-dihydroindolo[2,3-a]carbazole (10 g, 29.72 mmol), 5-bromo-2-iodopyridine (17 g, 59.44 mmol), Cu (3.4 g, 53.49 mmol), and Cs₂CO₃ (24 g, 74.20 mmol) in 1,2-dichlorobenzene 150 mL in a flask, the mixture was refluxed at 220° C. for 4 hours. After the reaction is completed, the reactant was distilled, an organic layer was extracted with ethyl acetate, and remaining moisture was removed with magnesium sulfate and dried. The remaining product was then separated with column chromatography to obtain compound 10-1 (9.6 g, yield: 68%).

Preparation of Compound 10-2

After dissolving compound 10-1 (8.6 g, 17.46 mmol), diborane (6.6 g, 26.19 mmol), PdCl₂(PPh₃)₂ (0.61 g, 0.873 mmol), and KOAc (4.2 g, 43.65 mmol) in 1,4-dioxane 100 mL in a flask, the mixture was refluxed at 120° C. for 4 hours. After the reaction is completed, an organic layer was extracted with ethyl acetate, and remaining moisture was removed with magnesium sulfate and dried. The remaining product was then separated with column chromatography to obtain compound 10-2 (7.4 g, yield: 79%).

Preparation of Compound C-8

After dissolving compound 10-2 (7.4 g, 13.82 mmol), compound A (4.5 g, 16.58 mmol), K₂CO₃ (5.7 g, 41.46 mmol), and Pd(PPhs)₄ (0.80 g, 0.69 mmol) in a mixture solvent of 2 M K₂CO₃ 30 mL, toluene 70 mL, and EtOH 30 mL in a flask, the mixture was refluxed at 120° C. for 3 hours. After the reaction is completed, an organic layer was extracted with ethyl acetate, and remaining moisture was removed with magnesium sulfate and dried. The remaining product was then separated with column chromatography to obtain compound C-8 (3.9 g, yield: 44%).

¹H NMR (600 MHz, DMSO, δ) 9.476 (s, 1H), 8.823-8.785 (m, 5H), 8.361-8.318 (m, 3H), 8.281-8.268 (d, J=7.8 Hz, 1H), 7.758-7.734 (m, 3H), 7.698-7.673 (m, 4H), 7.451-7.315 (m, 5H), 7.252-7.233 (d, J=7.8 Hz, 1H), 7.171-7.132 (m, 3H), 6.984-6.971 (m, 2H)

| | MW | UV | PL | M.P. |
|---|---|---|---|---|
| C-8 | 640.73 | 420 nm | 495 nm | 254° C. |

### Example 11: Preparation of Compound C-36

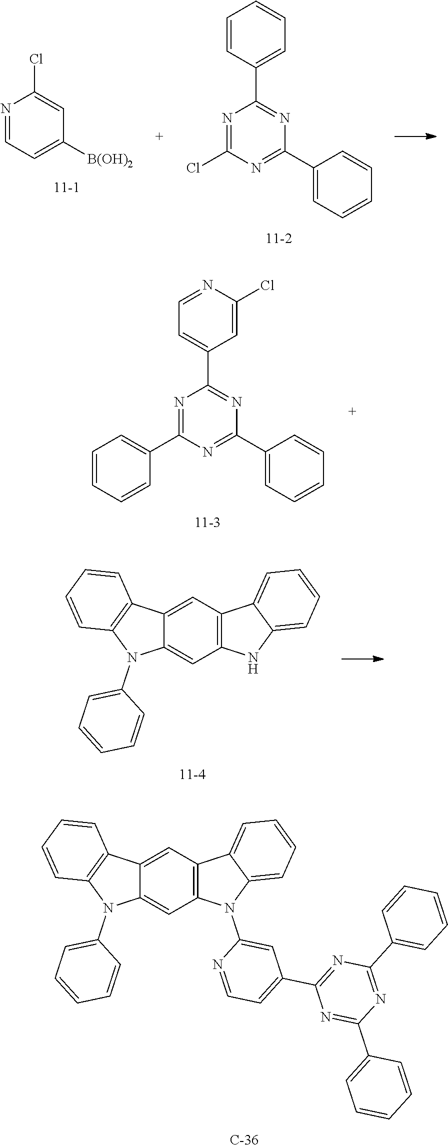

Preparation of Compound 11-3

After dissolving compound 11-1 (4 g, 25.4 mmol), compound 11-2 (10.2 g, 38.1 mmol), Pd(PPh₃)₄ (1.5 g, 1.3 mmol), and K₂CO₃ (7.0 g, 50.8 mmol) in a mixture solvent of toluene 130 mL, EtOH 35 mL, and H₂O 35 mL in a flask, the mixture was refluxed at 130° C. for 4 hours. After the reaction is completed, the reactant was extracted with EA, and the remaining product was then separated with column chromatography to obtain compound 11-3 (4.0 g, yield: 45%).

Preparation of Compound C-36

After dissolving compound 11-4 (3.0 g, 8.96 mmol), compound 11-3 (3.4 g, 9.86 mmol), Pd(OAc)₂ (100 mg, 0.45 mmol), S-phos (370 mg, 0.90 mmol), and NaOtBu (2.20 g, 22.4 mmol) in xylene 45 mL in a flask, the mixture was refluxed for 1 hour. The mixture was then cooled to room temperature, and MeOH was added thereto. The produced solid was filtered, and the filtrate was separated with column chromatography to obtain compound C-36 (3.5 g, yield: 61%).

¹H NMR (600 MHz, CDCl₃, δ) 9.00 (s, 1H), 8.89 (d, J=5.04 Hz, 1H), 8.84 (s, 1H), 8.76 (d, J=7.32 Hz, 4H), 8.50 (d, J=4.98 Hz, 1H), 8.27 (dd, J1=7.50 Hz, J2=4.92 Hz, 2H), 7.99 (d, J=8.10 Hz, 1H), 7.87 (s, 1H), 7.64-7.56 (m, 8H), 7.46 (t, J=7.50 Hz, 1H), 7.41-7.35 (m, 3H), 7.34-7.33 (m, 1H), 7.32-7.28 (m, 2H), 7.25-7.24 (m, 1H)

| | MW | UV | PL | M.P. |
|---|---|---|---|---|
| C-36 | 640.73 | 458.03 nm | 535.07 nm | 261.4° C. |

### Example 12: Preparation of Compound C-42

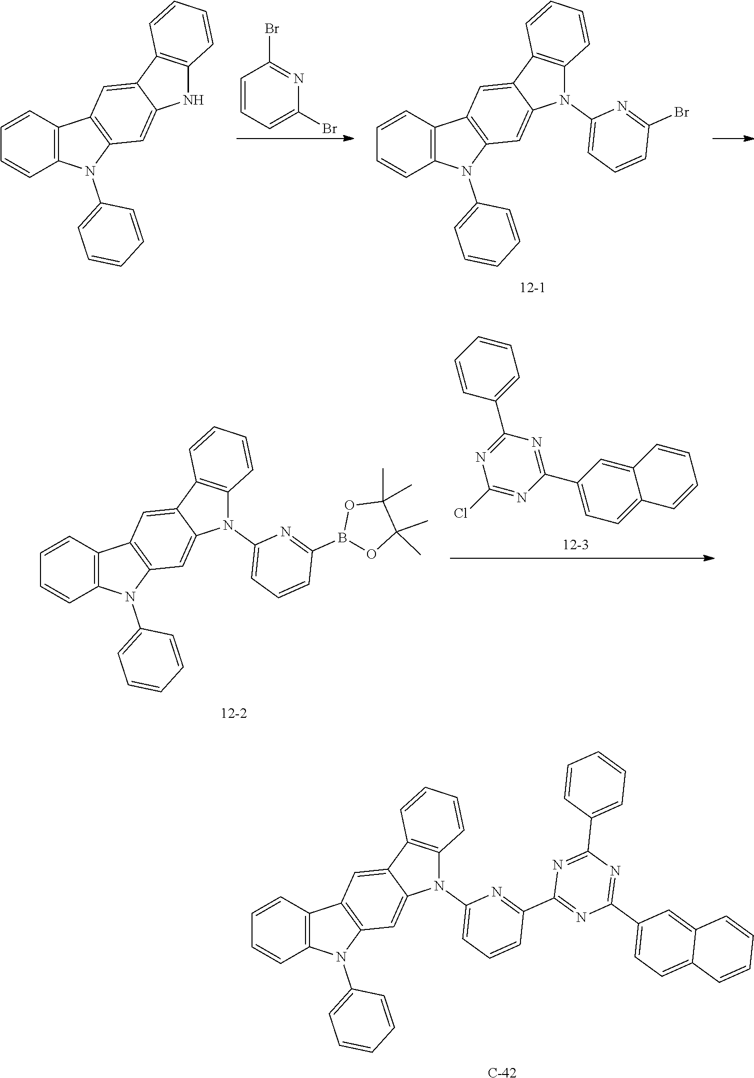

Preparation of Compound 12-1

After introducing 5-phenyl-5,7-dihydroindolo[2,3-b]carbazole (37.4 g, 112.6 mmol), 2,6-dibromopyridine (40 g, 168.8 mmol), CuI (10.7 g, 56.3 mmol), K₃PO₄ (71.7 g, 337.8 mmol), 1,2-ethylenediamine (12 mL, 168.8 mmol), and toluene 500 mL into a flask, the mixture was refluxed for 6 hours. After the reaction is completed, the mixture was cooled to room temperature, extracted with dichloromethane and purified water, the extracted organic layer was distilled under reduced pressure, and the residue was separated with column chromatography to obtain compound 12-1 (44 g, yield: 80%).

Preparation of Compound 12-2

After introducing compound 12-1 (44 g, 90.1 mmol), pinacolato diboron (22.9 g, 90.1 mmol), PdCl₂(PPh₃)₂ (6 g, 9 mmol), potassium acetate (22 g, 225 mmol), and 1,4-dioxane 500 mL into a flask, the mixture was stirred under reflux for 3 hours. The mixture was then extracted with EA and purified water, the extracted organic layer was distilled under reduced pressure, and the residue was separated with column chromatography to obtain compound 12-2 (31 g, yield: 64.3%).

Preparation of Compound C-42

After introducing compound 12-2 (8.3 g, 15.5 mmol), compound 12-3 (3.3 g, 10.3 mmol), Pd(OAc)₂ (116 mg, 0.516 mmol), S-Phos (425 mg, 0.103 mmol), Cs₂CO₃ (8.4 g, 25.8 mmol), CuCl (1.02 g, 10.3 mmol), and 1,4-dioxane 80 mL into a flask, the mixture was refluxed for 3 hours. The mixture was then extracted with EA and purified water, the extracted organic layer was distilled under reduced pressure, and the residue was separated with column chromatography to obtain compound C-42 (2.3 g, yield: 32%).

¹H NMR (600 MHz, CDCl₃, δ) 9.4 (s, 1H), 8.9-8.85 (m, 4H), 8.8 (d, 1H), 8.39 (d, 1H), 8.3 (m, 2H), 8.18 (s, 1H), 8.16-8.13 (t, 1H), 8.05 (d, 1H), 8.02-8.0 (d, 1H), 7.95-7.90 (d, 2H), 7.7-7.59 (m, 4h), 7.58-7.52 (m, 4h), 7.45-7.41 (t, 1h), 7.40-7.35 (m, 2h), 7.35-7.30 (m, 1h), 7.25-7.20 (t, 2h), 7.21-7.18 (t, 1h)

| | MW | UV | PL | M.P. |
|---|---|---|---|---|
| C-42 | 690.25 | 344 nm | 519 nm | 174° C. |

### Device Example 1-1: Production of an OLED Device Comprising the Phosphorous Host Material According to the Present Invention

An OLED device was produced using the phosphorous host material according to the present invention. A transparent electrode indium tin oxide (ITO) thin film (10 Ω/sq) on a glass substrate for an organic light-emitting diode (OLED) device (Samsung-Corning, Korea) was subjected to an ultrasonic washing with trichloroethylene, acetone, ethanol, and distilled water, sequentially, and was then stored in isopropanol. Next, the ITO substrate was mounted on a substrate holder of a vacuum vapor depositing apparatus. Dipyrazino[2,3-f:2′,3′-h]quinoxaline-2,3,6,7,10,11-hexacarbonitrile (compound HI-1) was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 5 nm on the ITO substrate. N,N′-bis(naphthalen-1-yl)-N,N′-bis(phenyl)-benzidine (compound HI-2) was then introduced into another cell of said vacuum vapor depositing apparatus, and was evaporated by applying an electric current to the cell, thereby forming a second hole injection layer having a thickness of 95 nm on the first hole injection layer. N-([1,1′-biphenyl]-4-yl)-N-(4-(9-(dibenzo[b,d]furan-4-yl)-9H-fluoren-9-yl)phenyl)-[1,1′-biphenyl]-4-amine (compound HT-1) was introduced into another cell of said vacuum vapor depositing apparatus, and was evaporated by applying an electric current to the cell, thereby forming a first hole transport layer having a thickness of 20 nm on the second hole injection layer. Thereafter, compound C-1 was introduced into one cell of the vacuum vapor depositing apparatus as a host, and compound D-74 was introduced into another cell as a dopant. The two materials were evaporated at different rates and were deposited in a doping amount of 12 wt % (the amount of dopant) based on the total amount of the host and dopant to form a light-emitting layer having a thickness of 30 nm on the hole transport layer. 2,4,6-tris(9,9-dimethyl-9H-fluoren-2-yl)-1,3,5-triazine (compound ET-1) was then introduced into another cell, and deposited to form an electron transport layer having a thickness of 35 nm on the light-emitting layer. Next, after depositing 8-hydroxyquinolatolithium (compound EI-1) as an electron injection layer having a thickness of 2 nm on the electron transport layer, an Al cathode having a thickness of 80 nm was deposited by another vacuum vapor deposition apparatus on the electron injection layer. Thus, an OLED device was produced.

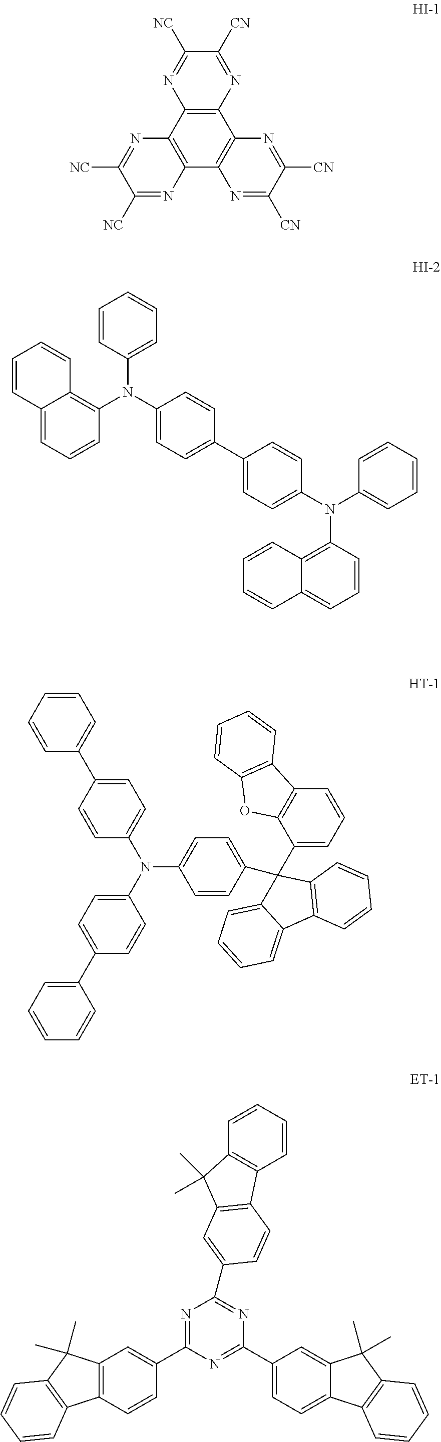 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(C4(c5cccc6c5oc5ccccc56)c5ccccc5-c5ccccc54)cc3)cc2)cc1 ; CC1(C)c2ccccc2-c2ccc(-c3nc(-c4ccc5c(c4)C(C)(C)c4ccccc4-5)nc(-c4ccc5c(c4)C(C)(C)c4ccccc4-5)n3)cc21]

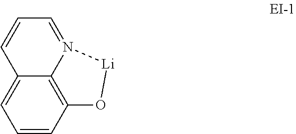

### Comparative Example 1-1: Production of an OLED Device Comprising a Conventional Phosphorous Host Material

An OLED device was produced in the same manner as in Device Example 1-1, except for using compound A-1 as a host of the light-emitting layer.

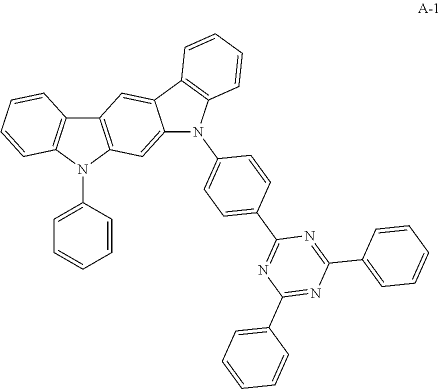 [SMILES: c1ccc(-c2nc(-c3ccccc3)nc(-c3ccc(-n4c5ccccc5c5cc6c7ccccc7n(-c7ccccc7)c6cc54)cc3)n2)cc1]

Time taken to be reduced from 100% to 97% of the luminance at 10,000 nit and a constant current of the OLEDs of Device Example 1-1 and Comparative Example 1-1 are shown in Table 1 below.

TABLE 1

| | | | T97 |
|---|---|---|---|
| | | | Lifespan |
| | Host | Dopant | [hrs] |
| Device | C-1 | D-74 | 56 |
| Example 1-1 | | | |
| Comparative | A-1 | D-74 | 41 |
| Example 1-1 | | | |

### Device Examples 1-2 to 1-4: Production of an OLED Device Coevaporating the Phosphorous Host Material and a Second Host Compound According to the Present Invention

An OLED device was produced using the phosphorous host material according to the present invention. A transparent electrode indium tin oxide (ITO) thin film (10 Ω/sq) on a glass substrate for an organic light-emitting diode (OLED) device (Geomatec, Japan) was subjected to an ultrasonic washing with acetone, ethanol, and distilled water, sequentially, and was then stored in isopropanol. Next, the ITO substrate was mounted on a substrate holder of a vacuum vapor depositing apparatus. Compound HI-3 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. Compound HI-1 was then introduced into another cell of said vacuum vapor depositing apparatus, and was evaporated by applying an electric current to the cell, thereby forming a second hole injection layer having a thickness of 5 nm on the first hole injection layer. Compound HT-2 was introduced into another cell of said vacuum vapor depositing apparatus, and was evaporated by applying an electric current to the cell, thereby forming a first hole transport layer having a thickness of 10 nm on the second hole injection layer. Compound HT-1 was introduced into another cell of said vacuum vapor depositing apparatus, and was evaporated by applying an electric current to the cell, thereby forming a second hole transport layer having a thickness of 30 nm on the first hole transport layer. Thereafter, the first host compound and second host compound of the Device Examples as listed in Table 2 below were introduced into two cells of the vacuum vapor depositing apparatus as hosts, and compound D-74 was introduced into another cell as a dopant. The two host materials were evaporated at the same rate of 1:1, and the dopant material was evaporated at a different rate and were deposited in a doping amount of 10 wt % (the amount of dopant) based on the total amount of the host and dopant to form a light-emitting layer having a thickness of 40 nm on the hole transport layer. Compound ET-2 and compound EI-1 were then introduced into another two cells, evaporated at the rate of 4:6, and deposited to form an electron transport layer having a thickness of 35 nm on the light-emitting layer. Next, after depositing compound EI-1 as an electron injection layer having a thickness of 2 nm on the electron transport layer, an Al cathode having a thickness of 80 nm was deposited by another vacuum vapor deposition apparatus on the electron injection layer. Thus, an OLED device was produced. All the materials used for producing the OLED device were those purified by vacuum sublimation at 10⁻⁶ torr.

Time taken to be reduced from 100% to 98.5% of the luminance at 10,000 nit and a constant current of the OLED is shown in Table 2 below.

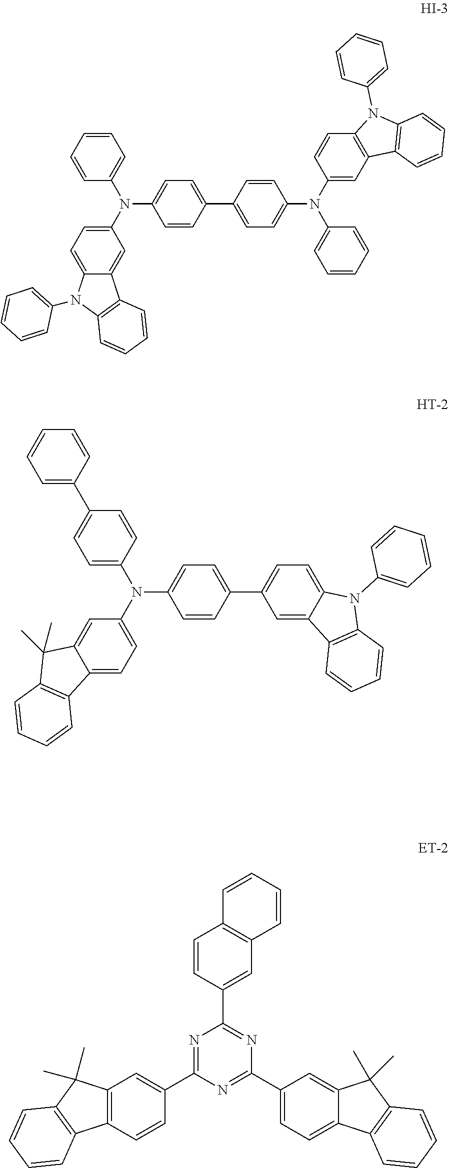 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(-c3nc(-c4ccc5c(c4)C(C)(C)c4ccccc4-5)nc(-c4ccc5ccccc5c4)n3)cc21]

### Comparative Examples 1-2 to 1-4: Production of an OLED Device Comprising a Conventional Phosphorous Host Material

An OLED device was produced in the same manner as in Device Example 1-2, except for using the first and second host compounds of the Comparative Examples as listed in Table 2 below as hosts of the light-emitting layer.

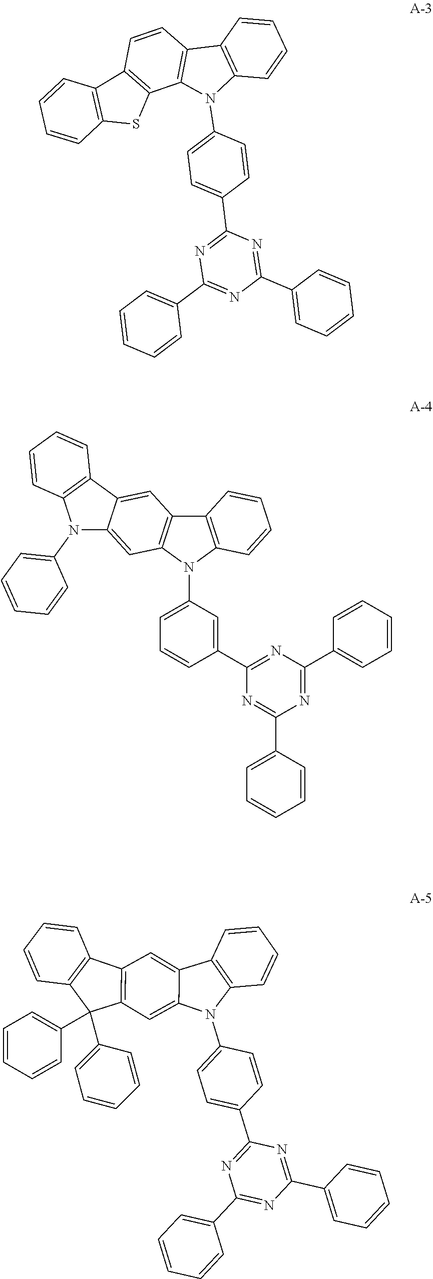 [SMILES: c1ccc(-c2nc(-c3ccccc3)nc(-c3ccc(-n4c5ccccc5c5ccc6c7ccccc7sc6c54)cc3)n2)cc1 ; c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1]

Time taken to be reduced from 100% to 98.5% of the luminance at 10,000 nit and a constant current of the OLED is shown in Table 2 below.

TABLE 2

| | | | T98.5 Lifespan |
|---|---|---|---|
| | Host | Dopant | [hrs] |
| Device | B-63:C-46 | D-74 | 74 |
| Example 1-2 | | | |
| Comparative | B-63:A-4 | D-74 | 33 |
| Example 1-2 | | | |
| Device | B-63:C-71 | D-74 | 41 |
| Example 1-3 | | | |
| Comparative | B-63:A-3 | D-74 | 14 |
| Example 1-3 | | | |
| Device | B-63:C-84 | D-74 | 33 |
| Example 1-4 | | | |
| Comparative | B-63:A-5 | D-74 | 15 |
| Example 1-4 | | | |

By comprising a specific host, the organic electroluminescent device of the present invention has longer lifespan than the conventional devices.

### Device Examples 2-1 to 2-6: Production of an OLED Device Comprising the Phosphorous Host Material According to the Present Invention

An OLED device was produced using the phosphorous host material according to the present invention. A transparent electrode indium tin oxide (ITO) thin film (10 Ω/sq) on a glass substrate for an organic light-emitting diode (OLED) device (Geomatec, Japan) was subjected to an ultrasonic washing with acetone, ethanol, and distilled water, sequentially, and was then stored in isopropanol. Next, the ITO substrate was mounted on a substrate holder of a vacuum vapor depositing apparatus. Compound HI-3 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. Compound HI-1 was then introduced into another cell of said vacuum vapor depositing apparatus, and was evaporated by applying an electric current to the cell, thereby forming a second hole injection layer having a thickness of 5 nm on the first hole injection layer. Compound HT-2 was introduced into another cell of said vacuum vapor depositing apparatus, and was evaporated by applying an electric current to the cell, thereby forming a first hole transport layer having a thickness of 10 nm on the second hole injection layer. Compound HT-3 was introduced into another cell of said vacuum vapor depositing apparatus, and was evaporated by applying an electric current to the cell, thereby forming a second hole transport layer having a thickness of 60 nm on the first hole transport layer. Thereafter, a host compound as listed in Table 3 below was introduced into one cell of the vacuum vapor depositing apparatus as a host, and a dopant compound as listed in Table 3 below was introduced into another cell as a dopant. The two materials were evaporated at different rates and were deposited in a doping amount of 3 wt % (the amount of dopant) based on the total amount of the host and dopant to form a light-emitting layer having a thickness of 40 nm on the second hole transport layer. Compound ET-2 and compound EI-1 were then introduced into another two cells, evaporated at the rate of 1:1, and deposited to form an electron transport layer having a thickness of 30 nm on the light-emitting layer. Next, after depositing compound EI-1 as an electron injection layer having a thickness of 2 nm on the electron transport layer, an Al cathode having a thickness of 80 nm was deposited by another vacuum vapor deposition apparatus on the electron injection layer. Thus, an OLED device was produced.

A driving voltage and efficiency properties were measured at 1,000 nit of luminance, red light was emitted, and time taken to be reduced from 100% to 97% of the luminance at 5,000 nit and a constant current of OLEDs are shown in Table 3 below.

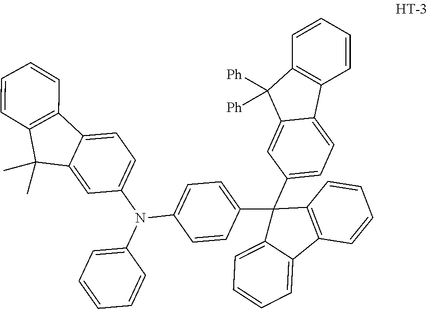 [SMILES: CC1(C)c2ccccc2-c2ccc(N(c3ccccc3)c3ccc(C4(c5ccc6c(c5)C(c5ccccc5)(c5ccccc5)c5ccccc5-6)c5ccccc5-c5ccccc54)cc3)cc21]

### Comparative Example 2-1: Production of an OLED Device Comprising a Conventional Phosphorous Host Material

An OLED device was produced in the same manner as in Device Examples 2-1 to 2-6, except for using compound A-2 instead of compound C-1 as a host of the light-emitting layer.

A driving voltage and efficiency properties were measured at 1,000 nit of luminance, red light was emitted, and time taken to be reduced from 100% to 97% of the luminance at 5,000 nit and a constant current of OLEDs are shown in Table 3 below.

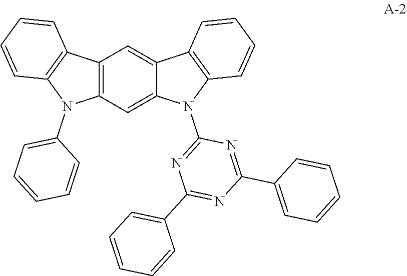 [SMILES: c1ccc(-c2nc(-c3ccccc3)nc(-n3c4ccccc4c4cc5c6ccccc6n(-c6ccccc6)c5cc43)n2)cc1]

TABLE 3

| | | | Driving | T97 |
|---|---|---|---|---|
| | | | voltage | Lifespan |
| | Host | Dopant | [V] | [hrs] |
| Device | C-1 | D-71 | 3.2 | 60 |
| Example 2-1 | | | | |
| Device | C-46 | D-71 | 3.2 | 36 |
| Example 2-2 | | | | |
| Device | C-56 | D-71 | 3.3 | 43 |
| Example 2-3 | | | | |
| Device | C-115 | D-71 | 4.0 | 17 |
| Example 2-4 | | | | |
| Device | C-36 | D-71 | 3.5 | 85 |
| Example 2-5 | | | | |
| Device | C-128 | D-71 | 3.3 | 86 |
| Example 2-6 | | | | |
| Comparative | A-2 | D-71 | 4.3 | 5 |
| Example 2-1 | | | | |

### Device Example 3-1: Production of an OLED Device Coevaporating the Phosphorous Host Material and a Second Host Compound According to the Present Invention

An OLED device was produced using the phosphorous host material according to the present invention. A transparent electrode indium tin oxide (ITO) thin film (10 Ω/sq) on a glass substrate for an organic light-emitting diode (OLED) device (Geomatec, Japan) was subjected to an ultrasonic washing with acetone, ethanol, and distilled water, sequentially, and was then stored in isopropanol. Next, the ITO substrate was mounted on a substrate holder of a vacuum vapor depositing apparatus. Compound HI-3 was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. Compound HI-1 was then introduced into another cell of said vacuum vapor depositing apparatus, and was evaporated by applying an electric current to the cell, thereby forming a second hole injection layer having a thickness of 5 nm on the first hole injection layer. Compound HT-2 was introduced into another cell of said vacuum vapor depositing apparatus, and was evaporated by applying an electric current to the cell, thereby forming a first hole transport layer having a thickness of 10 nm on the second hole injection layer. Compound HT-1 was introduced into another cell of said vacuum vapor depositing apparatus, and was evaporated by applying an electric current to the cell, thereby forming a second hole transport layer having a thickness of 30 nm on the first hole transport layer. Thereafter, compound B-10 and compound C-71 were introduced into two cells of the vacuum vapor depositing apparatus as hosts, and compound D-102 was introduced into another cell as a dopant. The two host materials were evaporated at the same rate of 1:1, and the dopant material was evaporated at a different rate and were deposited in a doping amount of 10 wt % (the amount of dopant) based on the total amount of the host and dopant to form a light-emitting layer having a thickness of 40 nm on the second hole transport layer. Compound ET-2 and compound EI-1 were then introduced into another two cells, evaporated at the rate of 4:6, and deposited to form an electron transport layer having a thickness of 35 nm on the light-emitting layer. Next, after depositing compound EI-1 as an electron injection layer having a thickness of 2 nm on the electron transport layer, an Al cathode having a thickness of 80 nm was deposited by another vacuum vapor deposition apparatus on the electron injection layer. Thus, an OLED device was produced. All the materials used for producing the OLED device were those purified by vacuum sublimation at 10⁻⁶ torr.

Time taken to be reduced from 100% to 97% of the luminance at 15,000 nit and a constant current of the OLED is shown in Table 4 below.

### Comparative Example 3-1: Production of an OLED Device Comprising a Conventional Phosphorous Host Material

An OLED device was produced in the same manner as in Device Example 3-1, except for using compound A-3 instead of compound C-71 as a host of the light-emitting layer.

Time taken to be reduced from 100% to 97% of the luminance at 15,000 nit and a constant current of the OLED is shown in Table 4 below.

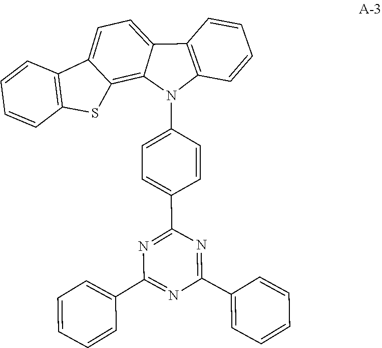 [SMILES: c1ccc(-c2nc(-c3ccccc3)nc(-c3ccc(-n4c5ccccc5c5ccc6c7ccccc7sc6c54)cc3)n2)cc1]

TABLE 4

| | | | T97 |
|---|---|---|---|
| | | | Lifespan |
| | Host | Dopant | [hrs] |
| Device | B-10:C-71 | D-102 | 41 |
| Example 3-1 | | | |
| Comparative | B-10:A-3 | D-102 | 25 |
| Example 3-1 | | | |

### Device Example 3-2: Production of an OLED Device Coevaporating the Phosphorous Host Material and a Second Host Compound According to the Present Invention

An OLED device was produced in the same manner as in Device Example 3-1, except for using compound C-46 instead of compound C-71 as a host of the light-emitting layer.

Time taken to be reduced from 100% to 95% of the luminance at 15,000 nit and a constant current of the OLED is shown in Table 5 below.

### Comparative Example 3-2: Production of an OLED Device Comprising a Conventional Phosphorous Host Material

An OLED device was produced in the same manner as in Device Example 3-2, except for using compound A-4 instead of compound C-46 as a host of the light-emitting layer.

Time taken to be reduced from 100% to 97% of the luminance at 15,000 nit and a constant current of the OLED is shown in Table 5 below.

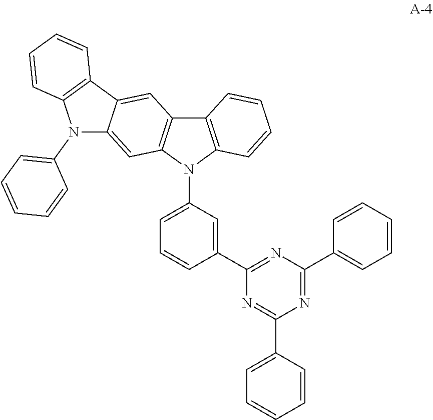 [SMILES: c1ccc(-c2nc(-c3ccccc3)nc(-c3cccc(-n4c5ccccc5c5cc6c7ccccc7n(-c7ccccc7)c6cc54)c3)n2)cc1]

TABLE 5

| | | | T95 |
|---|---|---|---|
| | | | Lifespan |
| | Host | Dopant | [hrs] |
| Device | B-10:C-46 | D-102 | 78 |
| Example 3-2 | | | |
| Comparative | B-10:A-4 | D-102 | 62 |
| Example 3-2 | | | |

### Device Example 3-3: Production of an OLED Device Coevaporating the Phosphorous Host Material and a Second Host Compound According to the Present Invention

An OLED device was produced in the same manner as in Device Example 3-1, except for using compound C-84 instead of compound C-71 as a host of the light-emitting layer.

Time taken to be reduced from 100% to 90% of the luminance at 15,000 nit and a constant current of the OLED is shown in Table 6 below.

### Comparative Example 3-3: Production of an OLED Device Comprising a Conventional Phosphorous Host Material

An OLED device was produced in the same manner as in Device Example 3-3, except for using compound A-5 instead of compound C-84 as a host of the light-emitting layer.

Time taken to be reduced from 100% to 90% of the luminance at 15,000 nit and a constant current of the OLED is shown in Table 6 below.

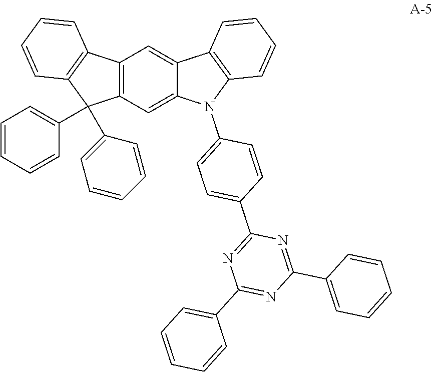 [SMILES: c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1]

TABLE 6

| | | | T90 |
|---|---|---|---|
| | | | Lifespan |
| | Host | Dopant | [hrs] |
| Device | B-10:C-84 | D-102 | 113 |
| Example 3-3 | | | |
| Comparative | B-10:A-5 | D-102 | 85 |
| Example 3-3 | | | |

By comprising a specific host, the organic electroluminescent device of the present invention has longer lifespan than conventional devices.

1. A phosphorous host material comprising a compound represented by the following formula (4): 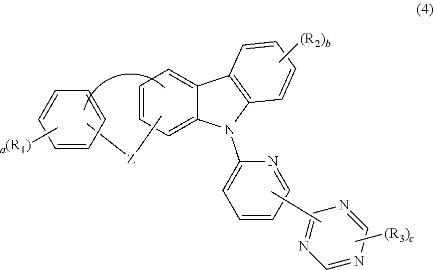
wherein
Z represents NR₄;
the structure of 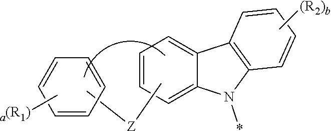
in formula (4) is represented formula (7): 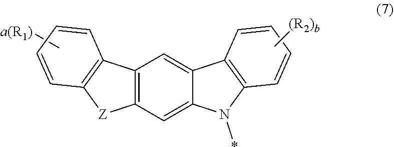
R₁ to R₄ each independently represent hydrogen, deuterium, a halogen, a cyano, a substituted or unsubstituted (C1-C30)alkyl, a substituted or unsubstituted (C6-C30)aryl, a substituted or unsubstituted 3- to 30-membered heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or are linked to an adjacent substituent(s) to form a substituted or unsubstituted, mono- or polycyclic, (C3-C30) alicyclic or aromatic ring, whose carbon atom(s) may be replaced with at least one hetero atom selected from nitrogen, oxygen, and sulfur;
a and b each independently represent an integer of 1 to 4;
c represents 1 or 2;
where a, b, or c is an integer of 2 or more, each of R₁, each of R₂, or each of R₃ may be the same or different; and
the heteroaryl contains at least one hetero atom selected from B, N, O, S, Si, and P.

2. The phosphorous host material according to claim 1, wherein the substituents of the substituted (C1-C30)alkyl, the substituted (C6-C30)aryl, the substituted 3- to 30-membered heteroaryl, the substituted (C3-C30)cycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, the substituted (C1-C30)alkyl(C6-C30)arylamino, and the substituted mono- or polycyclic, (C3-C30) alicyclic or aromatic ring in R₁ to R₈ each independently are at least one selected from the group consisting of deuterium, a halogen, a cyano, a carboxyl, a nitro, a hydroxyl, a (C1-C30)alkyl, a halo(C1-C30)alkyl, a (C2-C30) alkenyl, a (C2-C30) alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 5- to 30-membered heteroaryl unsubstituted or substituted with a (C6-C30)aryl, a (C6-C30)aryl unsubstituted or substituted with a 5- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

3. The phosphorous host material according to claim 1, wherein R₁ to R₄ each independently represent hydrogen, a substituted or unsubstituted (C1-C6)alkyl, or a substituted or unsubstituted (C6-C15)aryl; or are linked to an adjacent substituent(s) to form a substituted or unsubstituted, mono- or polycyclic, (C3-C12) aromatic ring.

4. The phosphorous host material according to claim 1, wherein
R₁ and R₂ each independently represent hydrogen; or are linked to an adjacent substituent(s) to form a substituted or unsubstituted, monocyclic (C3-C12) aromatic ring; and
R₃ and R₄ each independently represents an unsubstituted (C1-C6)alkyl, or a (C6-C15)aryl unsubstituted or substituted with a (C1-C6)alkyl.

5. The phosphorous host material according to claim 1, wherein the compound represented by formula 4 is selected from the group consisting of:

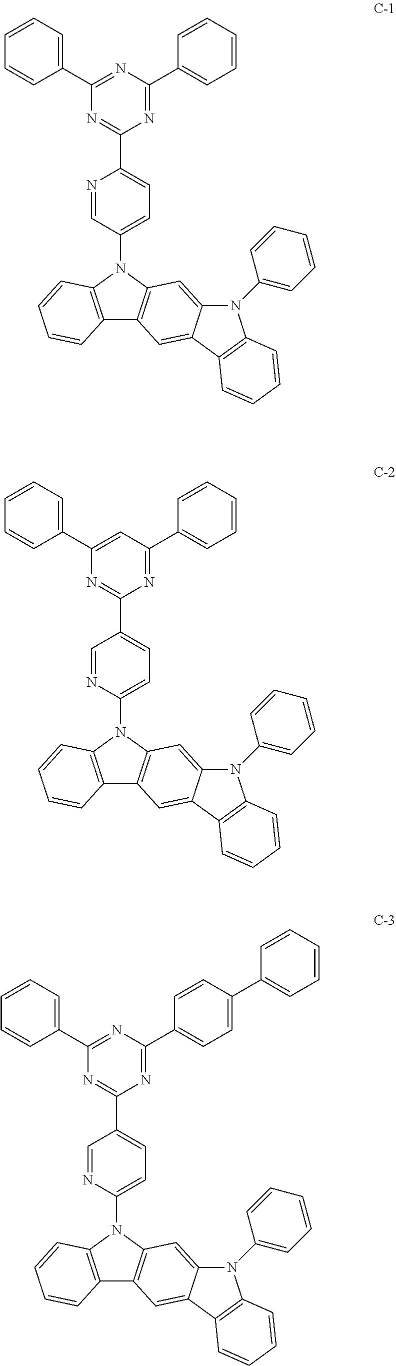 [SMILES: c1ccc(-c2nc(-c3ccccc3)nc(-c3ccc(-n4c5ccccc5c5cc6c7ccccc7n(-c7ccccc7)c6cc54)cn3)n2)cc1 ; c1ccc(-c2cc(-c3ccccc3)nc(-c3ccc(-n4c5ccccc5c5cc6c7ccccc7n(-c7ccccc7)c6cc54)nc3)n2)cc1 ; c1ccc(-c2ccc(-c3nc(-c4ccccc4)nc(-c4ccc(-n5c6ccccc6c6cc7c8ccccc8n(-c8ccccc8)c7cc65)nc4)n3)cc2)cc1]

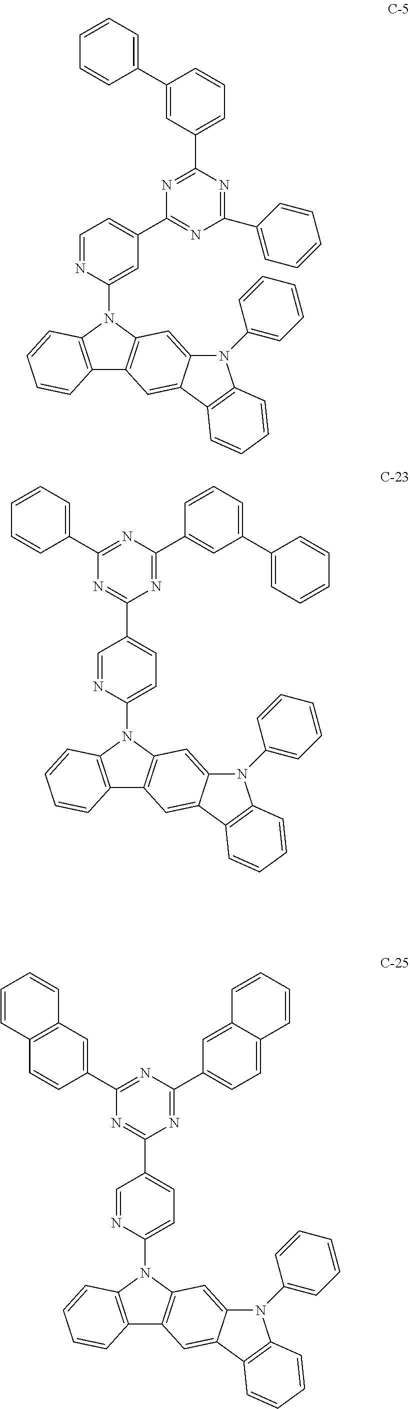

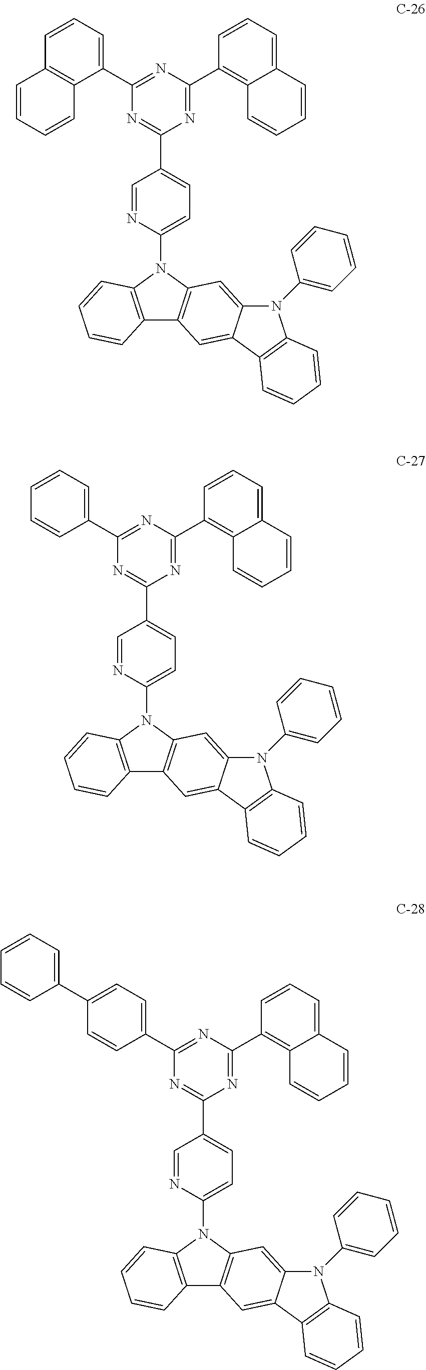 [SMILES: c1ccc(-n2c3ccccc3c3cc4c5ccccc5n(-c5ccc(-c6nc(-c7cccc8ccccc78)nc(-c7cccc8ccccc78)n6)cn5)c4cc32)cc1 ; c1ccc(-c2nc(-c3ccc(-n4c5ccccc5c5cc6c7ccccc7n(-c7ccccc7)c6cc54)nc3)nc(-c3cccc4ccccc34)n2)cc1 ; c1ccc(-c2ccc(-c3nc(-c4ccc(-n5c6ccccc6c6cc7c8ccccc8n(-c8ccccc8)c7cc65)nc4)nc(-c4cccc5ccccc45)n3)cc2)cc1]

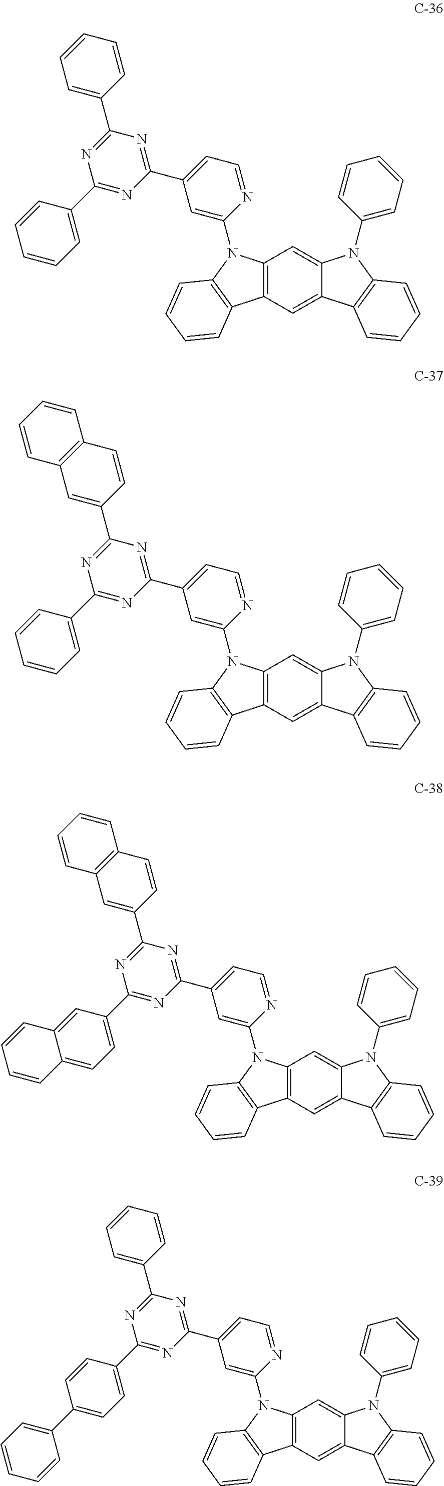 [SMILES: c1ccc(-c2nc(-c3ccccc3)nc(-c3ccnc(-n4c5ccccc5c5cc6c7ccccc7n(-c7ccccc7)c6cc54)c3)n2)cc1 ; c1ccc(-c2nc(-c3ccnc(-n4c5ccccc5c5cc6c7ccccc7n(-c7ccccc7)c6cc54)c3)nc(-c3ccc4ccccc4c3)n2)cc1 ; c1ccc(-n2c3ccccc3c3cc4c5ccccc5n(-c5cc(-c6nc(-c7ccc8ccccc8c7)nc(-c7ccc8ccccc8c7)n6)ccn5)c4cc32)cc1 ; c1ccc(-c2ccc(-c3nc(-c4ccccc4)nc(-c4ccnc(-n5c6ccccc6c6cc7c8ccccc8n(-c8ccccc8)c7cc65)c4)n3)cc2)cc1]

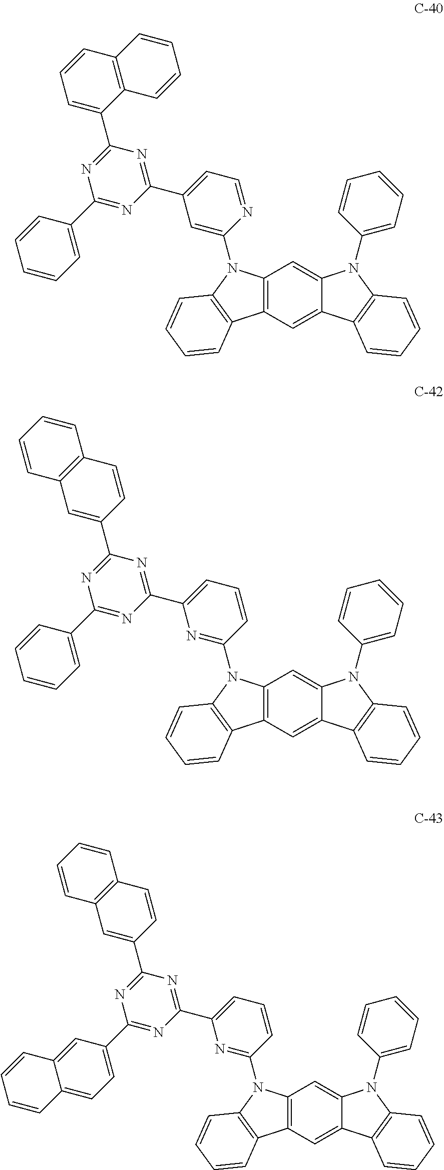 [SMILES: c1ccc(-c2nc(-c3ccnc(-n4c5ccccc5c5cc6c7ccccc7n(-c7ccccc7)c6cc54)c3)nc(-c3cccc4ccccc34)n2)cc1 ; c1ccc(-c2nc(-c3ccc4ccccc4c3)nc(-c3cccc(-n4c5ccccc5c5cc6c7ccccc7n(-c7ccccc7)c6cc54)n3)n2)cc1 ; c1ccc(-n2c3ccccc3c3cc4c5ccccc5n(-c5cccc(-c6nc(-c7ccc8ccccc8c7)nc(-c7ccc8ccccc8c7)n6)n5)c4cc32)cc1]

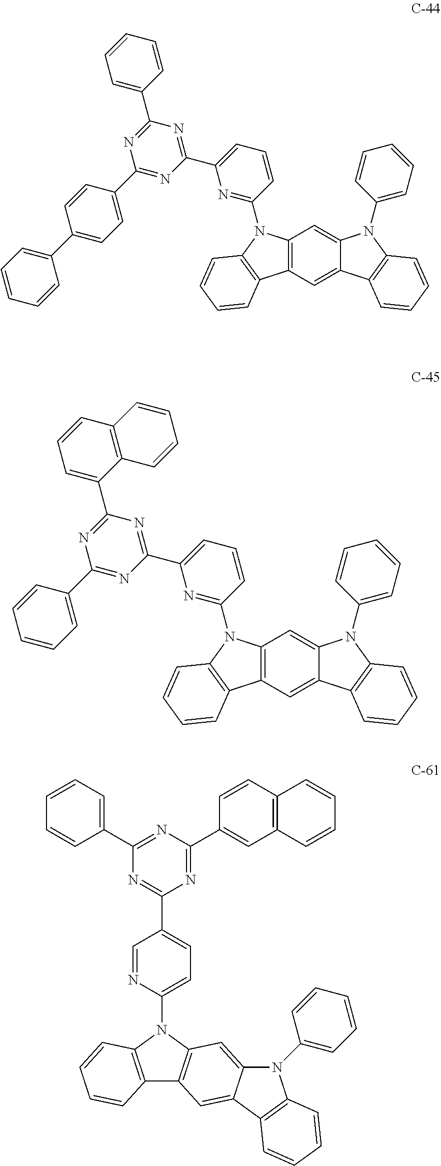 [SMILES: c1ccc(-c2ccc(-c3nc(-c4ccccc4)nc(-c4cccc(-n5c6ccccc6c6cc7c8ccccc8n(-c8ccccc8)c7cc65)n4)n3)cc2)cc1 ; c1ccc(-c2nc(-c3cccc(-n4c5ccccc5c5cc6c7ccccc7n(-c7ccccc7)c6cc54)n3)nc(-c3cccc4ccccc34)n2)cc1 ; c1ccc(-c2nc(-c3ccc(-n4c5ccccc5c5cc6c7ccccc7n(-c7ccccc7)c6cc54)nc3)nc(-c3ccc4ccccc4c3)n2)cc1]

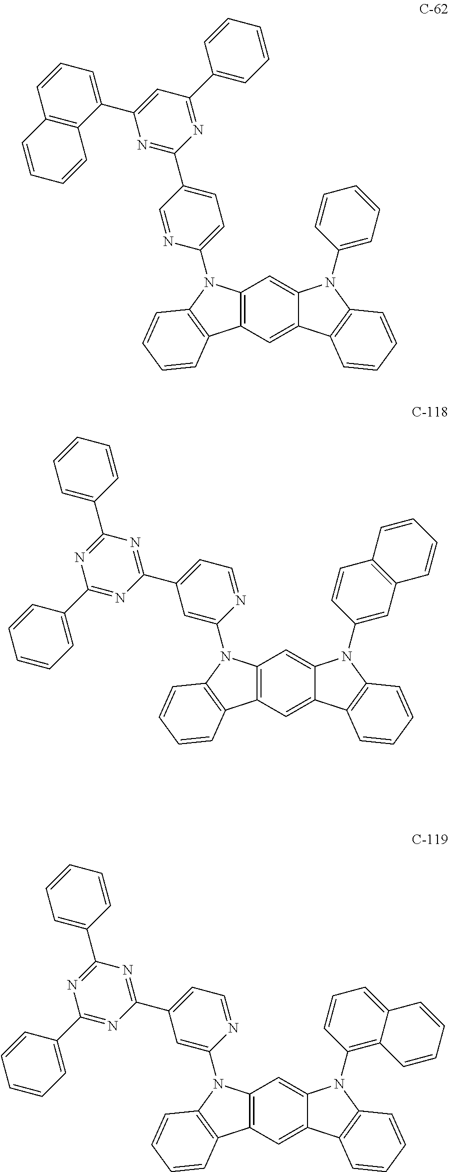 [SMILES: c1ccc(-c2cc(-c3cccc4ccccc34)nc(-c3ccc(-n4c5ccccc5c5cc6c7ccccc7n(-c7ccccc7)c6cc54)nc3)n2)cc1 ; c1ccc(-c2nc(-c3ccccc3)nc(-c3ccnc(-n4c5ccccc5c5cc6c7ccccc7n(-c7ccc8ccccc8c7)c6cc54)c3)n2)cc1 ; c1ccc(-c2nc(-c3ccccc3)nc(-c3ccnc(-n4c5ccccc5c5cc6c7ccccc7n(-c7cccc8ccccc78)c6cc54)c3)n2)cc1]

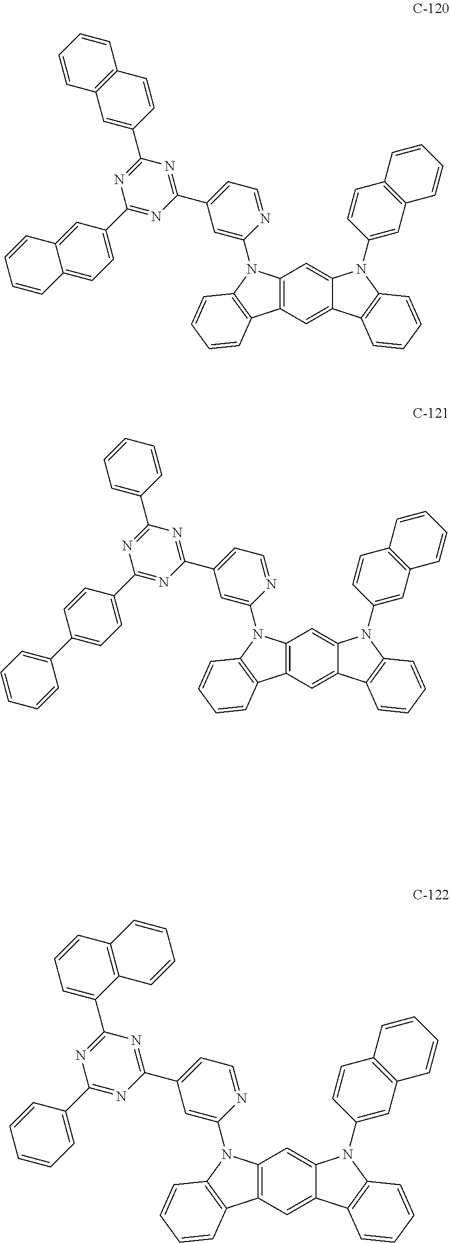 [SMILES: c1ccc2cc(-c3nc(-c4ccnc(-n5c6ccccc6c6cc7c8ccccc8n(-c8ccc9ccccc9c8)c7cc65)c4)nc(-c4ccc5ccccc5c4)n3)ccc2c1 ; c1ccc(-c2ccc(-c3nc(-c4ccccc4)nc(-c4ccnc(-n5c6ccccc6c6cc7c8ccccc8n(-c8ccc9ccccc9c8)c7cc65)c4)n3)cc2)cc1 ; c1ccc(-c2nc(-c3ccnc(-n4c5ccccc5c5cc6c7ccccc7n(-c7ccc8ccccc8c7)c6cc54)c3)nc(-c3cccc4ccccc34)n2)cc1]

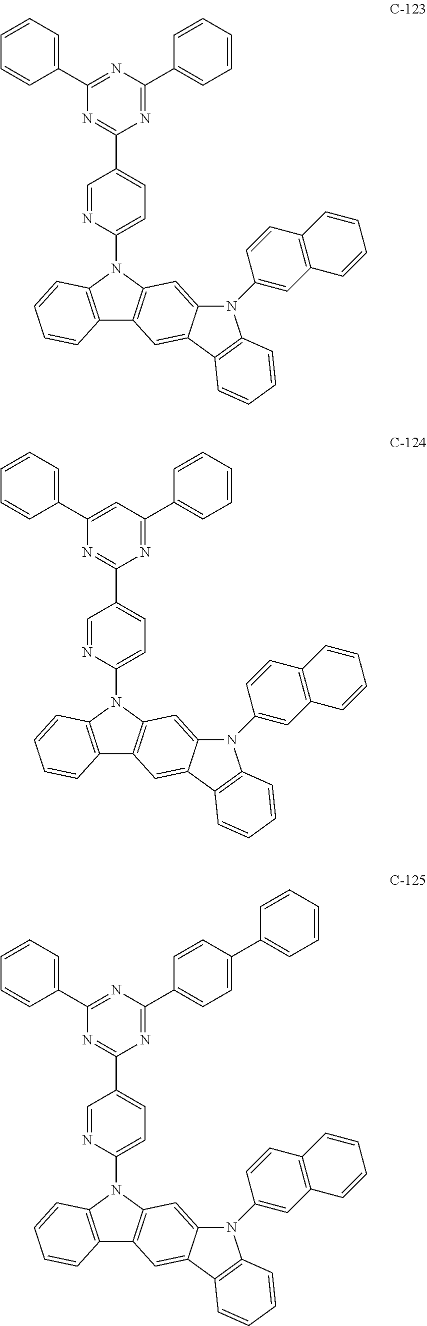 [SMILES: c1ccc(-c2nc(-c3ccccc3)nc(-c3ccc(-n4c5ccccc5c5cc6c7ccccc7n(-c7ccc8ccccc8c7)c6cc54)nc3)n2)cc1 ; c1ccc(-c2cc(-c3ccccc3)nc(-c3ccc(-n4c5ccccc5c5cc6c7ccccc7n(-c7ccc8ccccc8c7)c6cc54)nc3)n2)cc1 ; c1ccc(-c2ccc(-c3nc(-c4ccccc4)nc(-c4ccc(-n5c6ccccc6c6cc7c8ccccc8n(-c8ccc9ccccc9c8)c7cc65)nc4)n3)cc2)cc1]

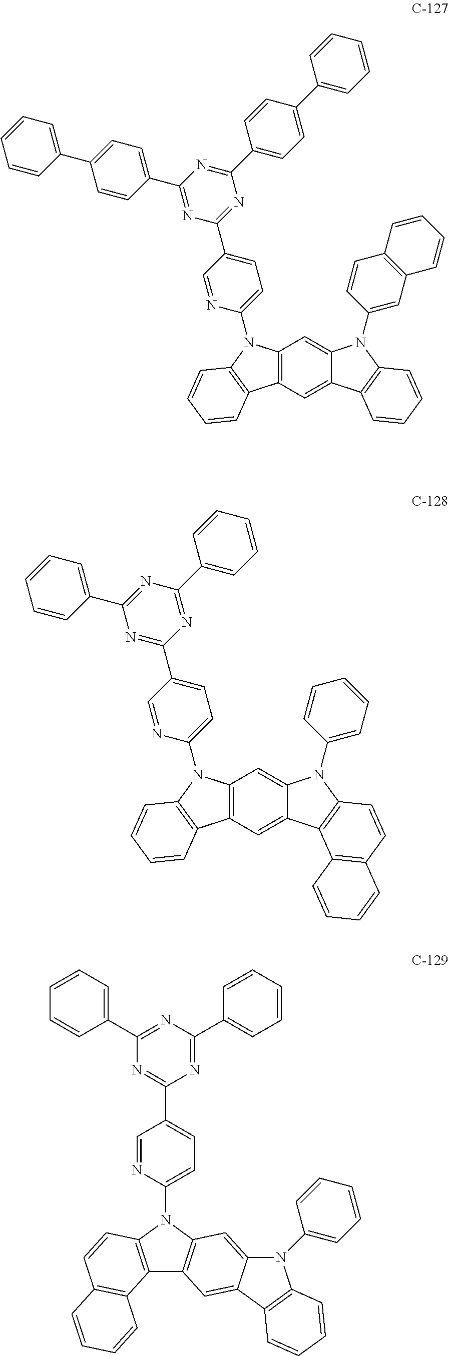 [SMILES: c1ccc(-c2ccc(-c3nc(-c4ccc(-c5ccccc5)cc4)nc(-c4ccc(-n5c6ccccc6c6cc7c8ccccc8n(-c8ccc9ccccc9c8)c7cc65)nc4)n3)cc2)cc1 ; c1ccc(-c2nc(-c3ccccc3)nc(-c3ccc(-n4c5ccccc5c5cc6c7c8ccccc8ccc7n(-c7ccccc7)c6cc54)nc3)n2)cc1 ; c1ccc(-c2nc(-c3ccccc3)nc(-c3ccc(-n4c5cc6c(cc5c5c7ccccc7ccc54)c4ccccc4n6-c4ccccc4)nc3)n2)cc1]

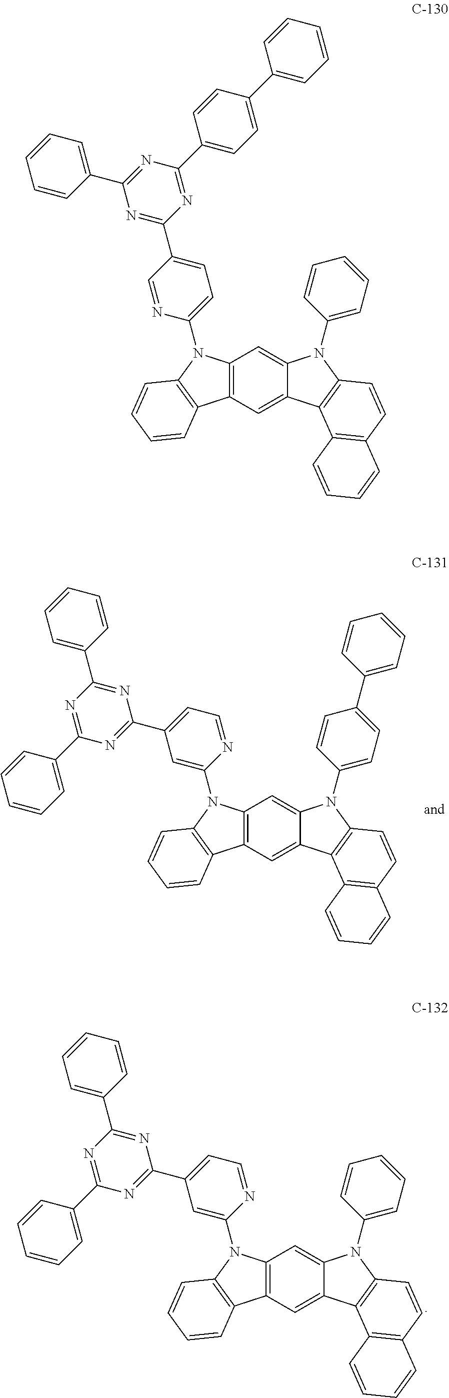 [SMILES: c1ccc(-c2ccc(-c3nc(-c4ccccc4)nc(-c4ccc(-n5c6ccccc6c6cc7c8c9ccccc9ccc8n(-c8ccccc8)c7cc65)nc4)n3)cc2)cc1 ; c1ccc(-c2ccc(-n3c4cc5c(cc4c4c6ccccc6ccc43)c3ccccc3n5-c3cc(-c4nc(-c5ccccc5)nc(-c5ccccc5)n4)ccn3)cc2)cc1 ; c1ccc(-c2nc(-c3ccccc3)nc(-c3ccnc(-n4c5ccccc5c5cc6c7c8ccccc8ccc7n(-c7ccccc7)c6cc54)c3)n2)cc1]

6. An organic electroluminescent device comprising the phosphorous host material according to claim 1.
